(19) United States
(12) Patent Application Publication
(10) Pub. No.: US 2024/0365774 A1
(43) Pub. Date: Nov. 7, 2024

(54) METHODS FOR CRYOPROTECTION AND LYOPROTECTION OF CELLS

(71) Applicant: LONZA LTD, Visp (CH)

(72) Inventors: Markus KARDORFF, Basel (CH); Dhananjay JERE, Basel (CH)

(21) Appl. No.: 18/573,245

(22) PCT Filed: Jun. 22, 2022
(86) PCT No.: PCT/EP2022/067054

§ 371 Date: Dec. 21, 2023

Related U.S. Application Data
(60) Provisional application No. 63/215,094, filed on Jun. 25, 2021.

(30) Foreign Application Priority Data
Aug. 23, 2021 (EP) 21192555.7

(51) Int. Cl. A01N 1/02

(52) U.S. Cl. CPC A01N 1/0221; A01N 1/0284

(57) ABSTRACT

Provided are compositions comprising a population of cells that are amenable to freezing or lyophilization while maintaining viable cells. The compositions comprise a monosaccharide, urea and, in some embodiments, a bulking agent. Also provided are methods for preparing and storing cells via freezing or lyophilization in a manner that allows cells to remain viable during storage.

### FIELD OF THE INVENTION

The present disclosure provides for methods of providing cryoprotection and/or lyoprotection of cells that are stored by freezing or lyophilization.

### BACKGROUND

Human mesenchymal stem cells (hMSCs) are important pluripotent stem cells with the ability to differentiate (in vitro) into different tissues such as fat, muscle, cartilage, bone, or neural cells [1-3]. The use of hMSCs in regenerative medicine and cell therapy is expanding [4, 5]. As hMSCs exhibit immunosuppressive capabilities [6], they have the potential to be harvested from an adult donor, expanded, modified, and subsequently used in allogenic treatments [5]. To enable this, hMSCs have to be stored for long periods of time. The current protocol for extended storage of hMSCs is by cryopreservation in liquid nitrogen [7]. However, storing cells in liquid nitrogen is cumbersome and expensive [8], especially for distribution and transport, as a regular supply of liquid nitrogen is needed to avoid uncontrolled thawing and damage to the cells [9].

Liquid nitrogen Storage of hMSCs also requires the addition of fetal bovine serum (FBS) and dimethyl sulfoxide (DMSO) to preserve the cells [7, 10]. DMSO is toxic [11-14] and can alter the expression of transcription factors and gene expression in hMSCs [15, 16]. Moreover, FBS is highly immunogenic in humans and could transfer pathogens [7]. Hence, DMSO and FBS are normally removed prior to administration to humans, a process which is labor intense and requires specially trained personnel and special facilities. There are therefore safety implications with a high risk of a sub-standard and inconsistent quality of the administered product.

It would also be beneficial to store a cell therapy product at refrigerated temperatures, similar to many other biological products, to facilitate easier handling and economical shipping. One possible way to achieve this would be to lyophilize (freeze-dry) the cells [18, 19]. Lyophilization is routinely applied to (drug) products that are unstable in liquid formulations, but stabilized in solid/dried form, such as vaccines, proteins, peptides or antibiotics [20].

EP 3 403 502 A1 discloses cryoprotectant and/or cryopreservant compositions, methods and uses thereof in the preservation and/or protection of cell, organism and/or tissue.

US 2009/123436 A1 discloses compositions for the cryogenic storage of biological materials and related methods.

WO 2021/050896 A1 discloses a method of producing a population of lyophilized cells, comprising: (a) freezing a composition comprising a population of cells, an aqueous component, a polyol, a sugar, and a polysaccharide; and (b) removing at least 90% of the aqueous component from the frozen composition to produce the population of lyophilized cells.

One object of the present invention is to provide methods for cryopreservation and lyophilization of cells that allow the cells to remain viable during storage.

### SUMMARY OF THE INVENTION

The present disclosure is directed to a composition comprising a population of viable cells, an aqueous component, urea and a monosaccharide.

In embodiments, the composition further comprises a bulking agent. In embodiments, the composition further comprises a hyaluronan gel.

In embodiments of the compositions, the concentration of the monosaccharide is from about 0.2 M to about 1.25 M, or from about 0.3 M to about 0.8 M, or from about 0.4 M to about 0.6 M.

In embodiments of the compositions, the concentration of the urea is from about 0.2 M to about 1.25 M, or from about 0.2 M to about 0.8 M, or from about 0.4 M to about 0.6 M.

In embodiments of the compositions, the concentration of the monosaccharide is from about 0.6 M to about 2.0 M, or from about 0.7 M to about 1.7 M, or from about 0.8 M to about 1.4 M.

In embodiments of the compositions, the concentration of the urea is from about 0.6 M to about 2.0 M, or from about 0.7 M to about 1.7 M, or from about 0.8 M to about 1.4 M.

In embodiments of the compositions, the molar ratio of the urea to monosaccharide in the composition being from about 5:1 to about 1:5, or from about 1:3 to about 3:1, or from about 1:2 to about 2:1, or is about 1:1.

In embodiments of the compositions, the monosaccharide is glucose, fructose or galactose.

In embodiments of the compositions, the bulking agent comprises a) a disaccharide, wherein the disaccharide is sucrose, lactose, maltose, trehalose, cellobiose, chitobiose, lactulose, isomaltose melibiose or gentiobiose; or b) a sugar alcohol, wherein the sugar alcohol is mannitol, sorbitol, galactitol, fucitol, iditol or inositol; or c) both a disaccharide and a sugar alcohol; or d) both sucrose and mannitol.

In embodiments of the compositions, the composition does not comprise DMSO, or, wherein the composition comprises DMSO at a concentration of from about 1% to about 10% prior to freezing, or wherein the composition comprises DMSO at a concentration of from about 1% to about 5% prior to freezing.

In embodiments of the compositions, the population of cells comprises mammalian cells.

In embodiments, the composition is in a frozen state at a temperature of between about −10° C. to about −100° C., or between about −20° C. to about −90° C., or between about −40° C. to about −60° C.

In embodiments, the composition is a lyophilized composition.

The composition according to anyone of claims 1 to 14, wherein the composition contains less than about 90% (vol/vol) of water.

In embodiments, the present disclosure is also directed to a method of producing a population of frozen cells, comprising a step (a) of freezing a composition as defined above to produce the population of frozen cells.

In embodiments, the present disclosure is also directed to a method of producing a population of reconstituted viable cells, comprising a step (a) of freezing a composition as defined above to produce a population of frozen cells and a step (b) of resuspending the population of frozen cells in a reconstitution agent to form a reconstituted composition, wherein at least about 1% of the cells are viable.

In embodiments, the present disclosure is also directed to a method of producing a population of lyophilized cells, comprising a step (a) of freezing a composition as defined above and a step (b) of removing at least about 10% (vol/vol) of water from the frozen composition to produce the population of lyophilized cells.

In embodiments, the present disclosure is also directed to a method of producing a population of reconstituted viable cells, comprising a step (a) of freezing a composition as defined above; a step (b) of removing at least about 10% (vol/vol) of water from the frozen composition to produce the population of lyophilized cells, and a step (c) of resuspending the population of lyophilized cells in a reconstitution agent to form a reconstituted composition, wherein at least about 1% of the cells are viable.

In embodiments of the methods above, prior to the freezing in step (a), the population of cells is isolated and contacted with a poration solution. In embodiments, the poration solution comprises trehalose. In embodiments, the concentration of trehalose in the poration solution is from about 0.1 M to about 1.0 M, or from about 0.1 M to about 0.6 M.

The present disclosure is also directed to a method of producing a population of frozen cells, comprising: freezing a composition comprising a population of cells, an aqueous component, urea, a monosaccharide, and optionally a bulking agent to produce the population of frozen cells.

The present disclosure is also directed to a method of producing a population of reconstituted viable cells, comprising: a) freezing a composition comprising a population of cells, an aqueous component, urea, a monosaccharide, and optionally a bulking agent to produce a population of frozen cells; and b) resuspending the population of frozen cells in a reconstitution agent to form a reconstituted composition, wherein at least about 1% of the cells are viable.

In embodiments of the methods, the composition further comprises a hydrogel. In embodiments, the hydrogel is a biocompatible hydrogel. In embodiments, the hydrogel is a hyaluronan gel, alginate gel, agarose gel, collagen gel or combination of thereof.

In embodiments, the population of cells is suspended in the hydrogel.

In embodiments of the methods, the concentration of the monosaccharide prior to freezing is from about 0.6 M to about 2 M, or from about 0.7 M to about 1.7 M, or from about 0.8 M to about 1.4 M.

In embodiments of the methods, the monosaccharide is glucose, fructose or galactose.

In embodiments of the methods, the monosaccharide is glucose and the concentration of the glucose prior to freezing is from about 0.2 M to about 1.0 M, or from about 0.2 M to about 0.8 M, or from about 0.4 M to about 0.6 M.

In embodiments of the methods, the concentration of the urea prior to freezing is from about 0.2 M to about 1.0 M, or from about 0.2 M to about 0.8 M, or from about 0.4 M to about 0.6 M.

In embodiments of the methods, the molar ratio of urea to monosaccharide prior to freezing is from about 1:3 to about 3:1, or from about 1:2 to about 2:1, or is about 1:1.

In embodiments of the methods, the monosaccharide is glucose and the molar ratio of urea to glucose prior to freezing is from about 1:3 to about 3:1, or from about 1:2 to about 2:1, or is about 1:1.

In embodiments of the methods, the freezing is performed with the cells in suspension or attached to a surface of a container, or with the population of cells suspended in a hydrogel, and/or in a container with or without collagen coating, optionally, wherein the container is a glass or plastic container with or without a membrane for cell attachment or growth.

The present disclosure is also directed to a method of producing a population of lyophilized cells, comprising: a) freezing a composition comprising a population of cells, a hyaluronan gel, an aqueous component, urea, a monosaccharide, and a bulking agent, wherein the concentration of the monosaccharide prior to freezing is at least about 0.6 M, and the concentration of urea prior to freezing is at least about 0.6 M; and b) removing at least about 90% of the aqueous component from the frozen composition to produce the population of lyophilized cells.

The present disclosure is also directed to a method of producing a population of reconstituted viable cells, comprising: a) freezing a composition comprising a population of cells, a hyaluronan gel, an aqueous component, urea, a monosaccharide, and a bulking agent, wherein the concentration of the monosaccharide prior to freezing is at least about 0.6 M, and the concentration of urea prior to freezing is at least about 0.6 M; b) removing at least about 90% of the aqueous component from the frozen composition to produce the population of lyophilized cells, and c) resuspending the population of lyophilized cells in a reconstitution agent to form a reconstituted composition, wherein at least about 1% of the cells are viable.

In embodiments of the methods, the population of cells is suspended in the hyaluronan gel.

In embodiments of the methods, the concentration of the monosaccharide prior to freezing is from about 0.6 M to about 2.0 M, or from about 0.7 M to about 1.7 M, or from about 0.8 M to about 1.4 M.

In embodiments of the methods, the monosaccharide is glucose, fructose or galactose.

In embodiments of the methods, the monosaccharide is glucose and the concentration of the glucose prior to freezing is from about 0.6 M to about 2.0 M, or from about 0.7 M to about 1.7 M, or from about 0.8 M to about 1.4 M.

In embodiments of the methods, the concentration of the urea prior to freezing is from about 0.6 M to about 2.0 M, or from about 0.7 M to about 1.7 M, or from about 0.8 M to about 1.4 M.

In embodiments of the methods, the molar ratio of urea to monosaccharide prior to freezing is from about 1:3 to about 3:1, or from about 1:2 to about 2:1, or is about 1:1.

In embodiments of the methods, the monosaccharide is glucose and the molar ratio of urea to glucose prior to freezing is from about 1:3 to about 3:1, or from about 1:2 to about 2:1, or is about 1:1.

In embodiments of the methods, the population of cells is from about 1×10⁴ cells per mL to 1×10⁷ cells per mL. In embodiments, the population of cells is from about 1×10⁵ cells per mL to 4×10⁵ cells per mL. In embodiments, the population of cells is from about 2×10⁵ cells per mL to 2.5×10⁵ cells per mL.

In embodiments of the methods, the bulking agent comprises a disaccharide. In embodiments, the concentration of disaccharide prior to freezing is from about 0.1 M to about 1.0 M. In embodiments, the concentration of disaccharide prior to freezing is from about 0.1 M to about 0.5 M. In embodiments, the concentration of disaccharide prior to freezing is from about 0.2 M to about 0.4 M. In embodiments, the disaccharide is sucrose, lactose, maltose, trehalose, cellobiose, chitobiose, lactulose, isomaltose melibiose or gentiobiose.

In embodiments, the disaccharide is sucrose. In embodiments, the concentration of sucrose prior to freezing is from about 0.1 M to about 1.0 M. In embodiments, the concentration of sucrose prior to freezing is from about 0.1 M to about 0.5 M. In embodiments, the concentration of sucrose prior to freezing is from about 0.2 M to about 0.4 M.

In embodiments of the methods, the bulking agent comprises a sugar alcohol. In embodiments, the concentration of sugar alcohol prior to freezing is from about 0.1 M to about 1.0 M. In embodiments, the concentration of sugar alcohol prior to freezing is from about 0.3 M to about 0.8 M. In embodiments, the concentration of sugar alcohol prior to freezing is from about 0.4 M to about 0.6 M.

In embodiments, the sugar alcohol is mannitol, sorbitol, galactitol, fucitol, iditol or inositol. In embodiments, the sugar alcohol is mannitol. In embodiments, the concentration of mannitol prior to freezing is from about 0.1 M to about 1.0 M. In embodiments, the concentration of mannitol prior to freezing is from about 0.3 M to about 0.8 M. In embodiments, the concentration of mannitol prior to freezing is from about 0.4 M to about 0.6 M.

In embodiments of the methods, the bulking agent comprises both a disaccharide and a sugar alcohol. In embodiments, the bulking agent comprises both sucrose and mannitol.

In embodiments of the methods, the composition does not comprise DMSO. In embodiments, the composition comprises DMSO at a concentration of from about 1% to about 10% prior to freezing. In embodiments, the composition comprises DMSO at a concentration of from about 1% to about 5% prior to freezing.

In embodiments of the methods, prior to the freezing in (a), the population of cells is isolated and contacted with a poration solution. In embodiments, the poration solution comprises trehalose. The poration solution may optionally further comprise cell culture medium. In embodiments, the concentration of trehalose in the poration solution is from about 0.1 M to about 1.0 M. In embodiments, the concentration of trehalose in the poration solution is from about 0.1 M to about 0.6 M.

In embodiments of the methods comprising reconstitution of cells, the reconstitution agent comprises cell culture medium. In embodiments, the reconstitution agent comprises a phosphate buffer solution.

In embodiments of the methods, the composition is an isotonic solution. In embodiments, the composition is a hypertonic solution.

In embodiments of the methods, the population of cells comprises mammalian cells. In embodiments, the population of cells comprises stem cells. In embodiments, the population of cells comprises pluripotent stem cells, embryonic stem cells, mesenchymal stem cells, or hematopoietic stem cells. In embodiments, the population of cells comprises mesenchymal stem cells. In embodiments, the population of cells comprises induced pluripotent stem cells. In embodiments, the population of cells comprises neuroblastoma cells. In embodiments, the neuroblastoma cells are SK-N-AS cells.

In the context of the present invention, the aqueous component is water and optionally comprises further components such as a buffer or cell culture medium.

In embodiments of the methods, the aqueous component comprises a buffer. In embodiments, the buffer comprises phosphate buffer, Tris buffer, acetate buffer, bicarbonate buffer, histidine buffer, citrate buffer, or combinations thereof. In embodiments, the aqueous component comprises cell culture medium. In embodiments, the cell culture medium is free of serum.

In embodiments of the methods, the removing the aqueous component comprises lowering pressure, applying heat, or both, to the frozen composition to remove the aqueous component. In embodiments, the removing the aqueous component comprises a primary drying step and a secondary drying step. In embodiments, the removing the aqueous component comprises only a primary drying step. In embodiments, the primary drying step comprises lowering pressure to remove the aqueous component. In embodiments, the secondary drying step comprises applying heat to remove the aqueous component.

In embodiments of the methods, the freezing occurs at between about −10° C. to about −100° C. In embodiments, the freezing occurs at between about −20° C. to about −90° C. In embodiments, the freezing occurs at between about −40° C. to about −60° C. In embodiments, the freezing lowers the temperature of the composition to about −80° C.

In embodiments of the methods, the freezing lowers the temperature of the composition to about −40° C., the aqueous component is removed at a pressure of a chamber pressure of about 60 mTorr to about 80 mTorr.

In embodiments of the methods, the resuspending the frozen cells occurs greater than two hours after freezing. In embodiments, the resuspending the frozen cells occurs greater than one day after freezing. In embodiments, the resuspending the frozen cells occurs greater than one week after freezing. In embodiments, the resuspending the frozen cells occurs greater than one month after freezing.

In embodiments where the cells are lyophilized, the resuspending the lyophilized cells occurs greater than two hours after the removing the aqueous component. In embodiments, the resuspending the lyophilized cells occurs greater than one day after the removing the aqueous component. In embodiments, the resuspending the lyophilized cells occurs greater than one week after the removing the aqueous component. In embodiments, the resuspending the lyophilized cells occurs greater than one month after the removing the aqueous component.

In embodiments of the methods, the frozen or lyophilized cells are stored below about −20° C. prior to the resuspending. In embodiments, the frozen or lyophilized cells are stored at about −20° C. to about 30° C. prior to the resuspending. In embodiments, the frozen or lyophilized cells are stored at about 4° C. to about 28° C. prior to the resuspending. In embodiments, the frozen or lyophilized cells are stored at about 10° C. to about 27° C. prior to the resuspending. In embodiments, the frozen or lyophilized cells are stored at about 2° C. to about 8° C. prior to the resuspending.

In embodiments of the methods, the frozen or lyophilized cells are stored at greater than about −20° C. for greater than 2 days prior to the resuspending. In embodiments, the lyophilized cells are stored at about 20° C. to about 25° C. for greater than 1 week prior to the resuspending. In embodiments, the lyophilized cells are stored at about 2° C. to about 8° C. for greater than 1 week prior to the resuspending.

In embodiments of the methods, at least about 20% of the cells in the reconstituted composition are viable, e.g., as measured by ALAMARBLUE® test. In embodiments, at least about 30% of the cells in the reconstituted composition are viable as measured by ALAMARBLUE® test. In embodiments, at least about 40% of the cells in the reconstituted composition are viable as measured by ALAMARBLUE® test. In embodiments, at least about 50% of the cells in the reconstituted composition are viable as measured by ALAMARBLUE® test.

In embodiments, the present disclosure is also directed to a composition comprising a population of viable cells, a hyaluronan gel, an aqueous component, urea, a monosaccharide, and a bulking agent, wherein urea and the monosaccharide are comprised in the composition at a molar ratio of about 1:3 to about 3:1, and wherein the composition contains less than about 90% (vol/vol) water.

In embodiments of the composition, the monosaccharide is glucose and urea and glucose are comprised in the composition at a molar ratio of about 1:3 to about 3:1. In embodiments of the composition, urea and glucose are comprised in the composition at a molar ratio of about 1:2 to about 2:1. In embodiments of the composition, urea and glucose are comprised in the composition at a molar ratio of about 1:1 to about 1:1.

In embodiments, the present disclosure is also directed to a composition comprising a population of viable cells, an aqueous component, urea, a monosaccharide, and optionally a bulking agent, wherein the molar ratio of the monosaccharide to urea in the composition is from about 5:1 to about 1:5. In embodiments, the molar ratio of the monosaccharide to urea in the composition is from about 3:1 to about 1:3. In embodiments, the molar ratio of the monosaccharide to urea in the composition is from about 2:1 to about 1:2. In embodiments, the molar ratio of the monosaccharide to urea in the composition is from about 1:1 to about 1:1.

In embodiments of the compositions, the monosaccharide is glucose. In embodiments, the molar ratio of glucose to urea in the composition is from about 3:1 to about 1:3. In embodiments, the molar ratio of glucose to urea in the composition is from about 2:1 to about 1:2. In embodiments, the molar ratio of glucose to urea in the composition is from about 1:1 to about 1:1.

In embodiments, the present disclosure is also directed to a composition comprising a population of viable cells, an aqueous component, from about 0.2 M to about 1.0 M urea, from about 0.2 M to about 1.0 M glucose, and a bulking agent. In embodiments, the composition comprises from about 0.3 M to about 0.8 M glucose. In embodiments, the composition comprises from about 0.4 M to about 0.6 M glucose.

In embodiments, the composition comprises from about 0.3 M to about 0.8 M urea. In embodiments, the composition comprises from about 0.4 M to about 0.6 M urea.

In embodiments, the composition further comprises sucrose. In embodiments, the composition further comprises from about 0.1 M to about 0.5 M sucrose.

In embodiments, the composition further comprises mannitol. In embodiments, the composition further comprises from about 0.1 M to about 0.8 M mannitol.

In embodiments of the compositions, the population of viable cells comprises mammalian cells. In embodiments, the population of viable cells comprises stem cells. In embodiments, the population of viable cells comprises pluripotent stem cells, embryonic stem cells, mesenchymal stem cells, or hematopoietic stem cells. In embodiments, the population of viable cells comprises mesenchymal stem cells. In embodiments, the population of viable cells comprises induced pluripotent stem cells. In embodiments, the population of viable cells comprises neuroblastoma cells. In embodiments, the neuroblastoma cells are SK-N-AS cells.

In embodiments of the compositions, the composition is free of DMSO. In embodiments of the compositions, the composition comprises DMSO at a concentration of from about 1% to about 10%. In embodiments of the compositions, the composition comprises DMSO at a concentration of from about 1% to about 5%.

In embodiments, the present disclosure is also directed to a method of freezing a population of cells, comprising freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.

In embodiments, the present disclosure is also directed to a method of lyophilizing a population of cells, comprising: a) freezing a composition comprising the population of cells, a hyaluronan gel, an aqueous component, about 0.6 M to about 2.0 M urea, about 0.6 M to about 2.0 M glucose, and about 0.1 M to about 1.0 M sucrose, wherein the freezing occurs below about −30° C.; and b) removing the aqueous component from the frozen composition to produce a population of lyophilized cells, wherein the aqueous component in the population of lyophilized cells is less than about 10% w/w.

In embodiments, the present disclosure is also directed to a method of producing a viable population of cells, comprising: a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and b) resuspending the population of lyophilized cells in a reconstitution agent to form a reconstituted composition, wherein at least about 1% of the cells in the reconstituted composition are viable.

In embodiments, the present disclosure is also directed to a method of producing a viable population of cells, comprising: a) freezing a composition comprising the population of cells, a hyaluronan gel, an aqueous component, about 0.6 M to about 2.0 M urea, about 0.6 M to about 2.0 M glucose, and about 0.1 M to about 1.0 M sucrose, wherein the freezing occurs below about −30° C.; b) removing the aqueous component from the frozen composition to produce a population of lyophilized cells, wherein the aqueous component in the population of lyophilized cells is less than about 10% w/w; and c) resuspending the population of lyophilized cells in a reconstitution agent to form a reconstituted composition, wherein at least about 1% of the cells in the reconstituted composition are viable.

In embodiments, the present disclosure is also directed to a method of freezing a population of cells, comprising freezing a composition comprising the population of cells, an aqueous component, about 0.2 M to about 1 M urea, about 0.2 M to about 1 M glucose, and about 0.1 M to about 0.8 M mannitol, wherein the freezing occurs below about −30° C.

In embodiments, the present disclosure is also directed to a method of lyophilizing a population of cells, comprising: a) freezing a composition comprising the population of cells, a hyaluronan gel, an aqueous component, about 0.6 M to about 2.0 M urea, about 0.6 M to about 2.0 M glucose, and about 0.1 M to about 0.8 M mannitol, wherein the freezing occurs below about −30° C.; and b) removing the aqueous component from the frozen composition to produce a population of lyophilized cells, wherein the aqueous component in the population of lyophilized cells is less than about 10% w/w.

In embodiments, the present disclosure is also directed to a method of producing a viable population of cells, comprising: a) freezing a composition comprising the population of cells, an aqueous component, about 0.2 M to about 1 M urea, about 0.2 M to about 1 M glucose, and about 0.1 M to about 0.8 M mannitol, wherein the freezing occurs below about −30° C.; and b) resuspending the population of lyophilized cells in a reconstitution agent to form a reconstituted composition, wherein at least about 1% of the cells in the reconstituted composition are viable.

In embodiments, the present disclosure is also directed to a method of producing a viable population of cells, comprising: a) freezing a composition comprising the population of cells, a hyaluronan gel, an aqueous component, about 0.6 M to about 2.0 M urea, about 0.6 M to about 2.0 M glucose, and about 0.1 M to about 0.8 M mannitol, wherein the freezing occurs below about −30° C.; b) removing the aqueous component from the frozen composition to produce a population of lyophilized cells, wherein the aqueous component in the population of lyophilized cells is less than about 10% w/w; and c) resuspending the population of lyophilized cells in a reconstitution agent to form a reconstituted composition, wherein at least about 1% of the cells in the reconstituted composition are viable.

In embodiments of the methods, the composition further comprises from about 1% to about 8% DMSO. In embodiments of the method, the composition further comprises a hydrogel. In embodiments, the hydrogel is a hyaluronan gel, alginate gel or collagen gel. In embodiments, the population of cells is suspended in the hydrogel. In embodiments, the composition is free of DMSO.

In embodiments of the methods where the composition is reconstituted, at least about 20% of the cells in the reconstituted composition are viable, e.g as measured by ALAMARBLUE® test. In embodiments, at least about 30% of the cells in the reconstituted composition are viable as measured by ALAMARBLUE® test. In embodiments, at least about 40% of the cells in the reconstituted composition are viable as measured by ALAMARBLUE® test. In embodiments, at least about 50% of the cells in the reconstituted composition are viable as measured by ALAMARBLUE® test.

### BRIEF DESCRIPTION OF THE DRAWINGS

The following drawings form part of the present specification and are included to further demonstrate exemplary embodiments of certain aspects of the present invention.

FIG. 1: FIGS. 1A-C are plots showing viability rates of hMSCs after freeze-thaw in different cryoprotective agent (CPA) formulations as described in Example 1. Each experiment was repeated five times with the bars indicating the average value and the error bars showing the standard deviation. Viability was assessed via metabolic conversion of a fluorescent dye. U=urea, G=glucose, DMSO=dimethyl sulfoxide, M=mannitol, S=sucrose. Cell viability is plotted as the percentage of viable cells. (FIG. 1A) While urea alone does not facilitate cryoprotection, glucose does. The two CPAs act in synergy and gain viabilities comparable to the DMSO control. (FIG. 1B) Evaluation of ratios of urea to glucose (adding to a total amount of 1 M CPAs). A 1:1 molar ratio is favorable. (FIG. 1C) Adding bulking agent to the U/G formulation also provided viable cells, despite increased osmotic stress. hMSCs were additionally incubated overnight in medium supplemented with 0.2 M trehalose prior to CPA application and freeze-thaw (FIG. 1C “With Tre Inc”). The pre-treatment slightly increased viabilities in the U/G/S formulation. The addition of 2% DMSO improves viabilities rates further. (FIG. 1D1 to 1D3) show microscopic images of hMSCs that underwent trehalose incubation and subsequent FT. The cells convert the fluorescent dye, are unaltered in morphology, and attached to the plastic surface, indicating viability. The scale bar is 200 μm.

FIG. 2 shows microscopic images of hMSCs proliferating after lyophilization in different formulations with high concentrations of CPAs, showing that hMSCs remain viable and can withstand the harsh conditions of the freeze-drying process as described in Example 1. U=urea, G=glucose, DMSO=dimethyl sulfoxide, M=mannitol, S=sucrose, Tre=trehalose. All cells were pre-incubated in trehalose before lyophilization. The scale bar is 400 μm.

### DETAILED DESCRIPTION OF THE INVENTION

The present disclosure provides methods of preparing and storing cells via freezing or lyophilization in a manner that allows cells to remain viable during storage. The present disclosure also provides compositions comprising a population of cells that are amenable to freezing or lyophilization while maintaining viable cells.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability, depending on the situation. In embodiments, one of skill in the art will understand the level of variability indicated by the term “about,” due to the context in which it is used herein. It should also be understood that use of the term “about” also includes the specifically recited value.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### Methods of Production a Population of Cells

In embodiments, the present disclosure provides methods of producing a population of frozen cells, comprising: freezing a composition comprising a population of cells, an aqueous component (e.g., water), urea, a monosaccharide, and optionally a bulking agent to produce the population of frozen cells.

In embodiments, the present disclosure provides methods of producing a population of reconstituted viable cells, comprising: a) freezing a composition comprising a population of cells, an aqueous component (e.g., water), urea, a monosaccharide, and optionally a bulking agent to produce a population of frozen cells; and b) resuspending the population of frozen cells in a reconstitution agent to form a reconstituted composition, wherein at least about 1% of the cells are viable.

In embodiments, the present disclosure provides methods of producing a population of lyophilized cells, comprising: a) freezing a composition comprising a population of cells, an aqueous component, urea, a monosaccharide, and a bulking agent; and b) removing at least about 90% of water from the frozen composition to produce the population of lyophilized cells.

In embodiments, the present disclosure provides methods of producing a population of reconstituted viable cells, comprising: a) freezing a composition comprising a population of cells, an aqueous component, urea, a monosaccharide, and a bulking agent; b) removing at least about 90% of water from the frozen composition to produce the population of lyophilized cells, and c) resuspending the population of lyophilized cells in a reconstitution agent to form a reconstituted composition, wherein at least about 1% of the cells are viable.

### Freezing and Lyophilization

In embodiments, freezing is the transition of the substance, e.g., a composition comprising one or more components, from liquid state to solid state. “Lyophilization” (also termed “freeze-drying” and “cryodesiccation”) and variants thereof broadly refers to freezing a substance and then reducing the concentration of one of the solutes, typically water, by sublimation and desorption (i.e., “drying”). In embodiments, the drying comprises applying vacuum to the frozen substance, e.g., frozen composition. In embodiments, the drying comprises lowering pressure, applying a vacuum, or both, to the frozen composition.

In embodiments of the methods provided herein involve freezing of the composition as part of the lyophilization process. In embodiments of the methods provided herein involve freezing of the composition but do not involve any drying steps. Any of the apparatuses and methods described herein for freezing compositions can be used either in the methods of only freezing provided herein or in the methods of lyophilization provided herein.

Lyophilization can be used for drying of thermally labile compounds, such as microorganisms and proteins. Instead of using evaporation mechanism of solvent elimination, which is common with small molecules, it employs sublimation. Lyophilization can consist of three process steps: freezing, primary drying and secondary drying. Lyophilization can be accomplished by placing the composition to be lyophilized in a lyophilization chamber, where the freezing, drying and ultimately stoppering under inert atmosphere takes place. During drying, water vapor can pass through a connecting duct into a condenser. In some embodiments, the temperature in the condenser is lower than the temperature of the lyophilization chamber such that the water vapor turns into ice.

### Freezing

Freezing can have a large impact on the rate and homogeneity of drying. Freezing of the solvent can be subdivided into cooling, phase change and solidification. In some embodiments, the formulation does not freeze at the equilibrium freezing temperature, but at a lower one. This is either because no ice crystal nuclei are yet formed in the solution, or because there are appreciable temperature differences within the system. The degree at which the phase change occurs, starting with primary and then secondary nucleation, is called the degree of supercooling. Primary nucleation is defined as the appearance of the first ice nucleus, while secondary nucleation is characterized by formation of additional nucleation sites. After the phase change stage, the solidification starts. Here the ice crystals start growing and the amount of liquid water phase is reduced, leading to increasing solute concentration. When the critical concentration is exceeded, the concentrated solution undergoes either eutectic freezing or vitrification. The temperature at which this occurs is called the eutectic temperature (Tₑᵤ) for crystalline systems and the glass transition temperature of the maximally freeze-concentrated solution (T_{g}′) for amorphous systems. Below these temperatures the system is considered completely solidified. Different crystal sizes and morphologies form depending on the degree of supercooling. This, upon sublimation during drying step, affects the pore sizes in the dry products. Small ice crystals form smaller pores, which causes higher resistance to mass transfer and therefore slower primary drying. However, because of larger surface area, the secondary drying in this case, can proceed with a higher rate. Vice versa can be true for larger ice crystals. In some embodiment, faster crystallization, which leads to smaller crystals, causes slower primary drying but faster secondary drying.

### Primary Drying

In embodiments of the lyophilization methods, the methods include a primary drying step. After the formulation is in its frozen state, the chamber is evacuated to a pressure close to vacuum and the temperature of the shelf is slowly increased to a temperature close, but below, collapse temperature (T_{c}). T_{c} is usually several degrees higher than T_{g}′ and is a temperature, where the cake loses its structure. The correlation between the collapsed cake and product stability is still debatable. However, visual collapse may cause the lyophilized cell product to be rejected after visual inspection by a manufacturer.

The driving force of primary drying is the gradient between ice vapor pressure and chamber pressure. The vapor pressure of ice is dependent on the temperature of the product, which is the most important parameter in the process. The product temperature is controlled by both changing the shelf temperature and chamber pressure. The higher shelf temperature leads to higher product temperature and hence to faster sublimation. On the other hand, since sublimation is an endothermic process, a lower chamber pressure leads to cooling of the product, which causes a lower rate of sublimation.

When drying cells, it may be important to use protectants against water loss and low temperatures in addition to bulking agents. Protectants can include sorbitol, sucrose, trehalose, mannitol, polyvinylpyrrolidone (PVP), dextrose and glycine. Most of these compounds have an additional positive effect of increasing T_{g}′ of the formulation. This is desirable, because the process can be run at higher temperatures, which makes it faster and therefore more efficient. Bulking agents provide a nice appearance and structure of the lyophilized cell product and some also serve as protectants—mannitol is an example of this. Crystalline bulking agents are desirable, because they tend to provide elegant cake structures.

### Secondary Drying

In embodiments of the lyophilization methods, the methods include a secondary drying step. In other embodiments, a secondary drying step is not performed. Free ice is sublimated during primary drying, which is the longest step in the lyophilization process. However, some of the water remains bound to the product and therefore needs to be removed with a more aggressive treatment. This is achieved during secondary drying, where the chamber pressure remains the same as in primary drying and shelf temperature is increased typically to 30-50° C. At this point, it is possible to raise the temperature to such levels, because there is no more free ice in the vial and so there is no danger of collapse of the lyophilization cake.

In embodiments, the freezing of the composition comprises placing containers containing the composition in liquid nitrogen. In embodiments, the freezing of the composition comprises placing containers containing the composition in a −80° C. freezer. In embodiments, the freezing of the composition comprises placing containers comprising the composition in a freezing container, e.g., the MR. FROSTY Freezing Container from THEFRMO SCIENTIFIC. In embodiments, the freezing of the composition comprises placing containers comprising the composition on a pre-cooled shelf in a freezer. In embodiments, the containers comprise a cryogenic vial or an injection glass vial, e.g., a 2R glass vial. In embodiments, the freezing of the composition does not use liquid nitrogen.

In embodiments, the rate at which the temperature of the composition comprising a population of cells is lowered can affect the viability of the cells. The rate of cooling will be dependent on numerous factors, including the cooling apparatus, the container, the composition, and the volume of the composition. In embodiments, the temperature of the composition is reduced about 1° C. to about 50° C. per second, about 2° C. to about 40° C. per second, about 3° C. to about 30° C. per second, about 4° C. to about 20° C. per second, or about 5° C. to about 10° C. per second. In embodiments, the temperature of the composition is reduced about 1° C. to about 50° C. per minute, about 2° C. to about 40° C. per minute, about 3° C. to about 30° C. per minute, about 4° C. to about 20° C. per minute, or about 5° C. to about 10° C. per minute. In embodiments, the temperature of the composition comprising a population of cells does not exceed the T_{g} of the composition during the freezing process.

In some embodiments, the removing the aqueous component, in particular water, in the method comprises lowering pressure, applying heat, or both, to the frozen composition to remove the aqueous component. In embodiments, the lowering pressure comprises applying vacuum to the frozen composition. In embodiments, the lowering pressure comprises lowering the pressure to 10 torr, e.g. less than about 8 torr, less than about 5 torr, less than about 2 torr, or less than about 1 torr. In embodiments, the lowering pressure comprises lowering the pressure to less than about 0.8 torr. In embodiments, the lowering pressure comprises lowering the pressure to less than about 0.4 torr.

In some embodiments, the removing the aqueous component, in particular water, comprises a primary drying step and a secondary drying step. In embodiments, the primary drying step comprises lowering pressure to remove water. In embodiments, the secondary drying step comprises applying heat to remove the aqueous component. In embodiments, the primary drying step comprises sublimation (i.e., transition from solid phase to gas phase) of water of the composition. In embodiments, the secondary drying step comprises desorption of water of the composition. When used in the context of lyophilization, “desorption” refers to the disruption of physio-chemical interactions between the aqueous component (e.g., water molecules) and one or more components of the frozen composition. In embodiments, the pressure and temperature are selected such that water of the composition is capable of sublimation and/or desorption.

In some embodiments, the applying heat in the secondary drying step comprises heating the composition to greater than −20° C. In embodiments, the applying heat comprises heating the composition to greater than −10° C. In embodiments, the applying heat comprises heating the composition to greater than 0° C.

In some embodiments, the lyophilization removes the solute, e.g., water or aqueous component, from the composition. One of skill in the art will appreciate that “lyophilized cells” may still comprise some amount of water. In embodiments, the amount of solute remaining in the population of lyophilized cells can be a factor in the cells maintaining their viability when the cells are reconstituted. In embodiments, the population of lyophilized cell comprises less than 10% w/w of water. In embodiments, the population of lyophilized cell comprises less than 5% w/w of water. In embodiments, the population of lyophilized cell comprises less than 10%, less than 9%, less than 8%, less than 7%, less than 6%, less than 5%, less than 4%, less than 3%, less than 2%, or less than 1% w/w of water. In embodiments, the composition comprising a lyophilized mixture of a cell and a lyophilization agent selected from glycerol, propylene glycol, or combinations thereof comprise less than 10% of water. In embodiments, the composition comprising a lyophilized mixture of a cell and a lyophilization agent selected from glycerol, propylene glycol, or combinations thereof comprise less than 5% of water. In embodiments, the composition comprising a lyophilized mixture of a cell and a lyophilization agent selected from glycerol, propylene glycol, or combinations thereof comprise less than 10%, less than 9%, less than 8%, less than 7%, less than 6%, less than 5%, less than 4%, less than 3%, less than 2%, or less than 1% w/w of water.

In other embodiments, the composition contains after lyophilization less than about 90% sometimes less than about 80%, sometimes less than about 70%, sometimes les than about 60% sometimes les than about 50%, sometimes less than about 40%, sometimes less than about 30% sometimes less than about 20%; sometimes less than about 10%, sometimes less than about 9%, sometimes less than about 8%, sometimes less than about 7%, sometimes less than about 6%, sometimes less than about 5%, sometimes less than about 4%, sometimes less than about 3%, sometimes less than about 2%, or sometimes less than about 1% (vol/vol) of water.

In some embodiments, the lyophilization is performed in a device capable of both freezing the composition and removing water, e.g., a freeze-dryer such as the FREEZONE freeze dryer from LABCONCO, the FREEZEMOBILE, VIRTIS, and HULL freeze dryers from SP SCIENTIFIC, and the STELLAR, REVO, MAGNUM, and EPIC freeze dryers from MILLROCK TECHNOLOGY. In embodiments, the freezing the composition and the removing the aqueous component are performed in separate devices, e.g., a freezer, e.g., −80° C. freezer, or liquid nitrogen for freezing the composition, and a vacuum system for removing water.

In some embodiments, the cells are lyophilized on a 2-dimensional surface, e.g., on the surface of a plate or a container. In embodiments, the lyophilization is performed on a surface, e.g., in a container, with or without collagen coating. In embodiments, the surface, e.g., the container, is a glass or plastic surface/container without or without membrane for cell attachment or growth.

In some embodiments, the cells are lyophilized on a 3-dimensional matrix. In embodiments, the lyophilization is performed on a 3D matrix in a container, e.g., with or without collagen coating.

In some embodiments, the composition comprising cells, an aqueous component, and a lyophilization agent further comprises a matrix. Non-limiting examples of matrices include, e.g., collagen (such as, e.g., Type I, Type II, or Type IV collagen), elastin, fibronectin, laminin, vitronectin, cadherin, tenascin-C, and other matrix-derived peptides. In embodiments, the population so cells are suspended in a matrix. In embodiments, the matrix is a hydrogel.

In embodiments of any of the above methods, the composition further comprises a hydrogel. A hydrogel is a network of polymer chains that are hydrophilic, sometimes found as a colloidal gel in which water is the dispersion medium. In embodiments, the hydrogel is a biocompatible hydrogel. In embodiments, the hydrogel is a hyaluronan gel, alginate gel, agarose gel, collagen gel, or combination thereof. In embodiments, the hydrogel is a combination of hyaluronan gel, alginate gel, agarose gel, or collagen gel. In embodiments, the population of cells are suspended in the hydrogel. In embodiments, the freezing of the population of cells is performed either in suspension or attached in a container. In embodiments, the freezing of the population of cells is performed with the population of cells suspended in a hydrogel. In embodiments, the hydrogel is a hyaluronan gel.

In some embodiments, the hydrogel is a HYSTEM™ Gel, available from Sigma Aldrich. HYSTEM™ can include HYSTEM™ which is composed of thiol modified HA (GLYCOSIL®) and thiol reactive crosslinker (EXTRALINK®). See, e.g., Chen D, et al., Eye (Lond), 2017 June; 31(6):962-971; Devarasetty M, et al., Biofabrication, 2017 Jun. 7; 9(2):021002; Mannino R G, et al., Lab Chip, 2017 Jan. 31; 17(3):407-414; Chen X, et al., J Tissue Eng Regen Med, 2016 May; 10(5):437-46; and Engel B J, et al., Adv Healthc Mater, 2015 Aug. 5; 4(11):1664-74. The crosslinker allows for the gelification process (without it, HA solution will stay liquid). HYSTEM™ can also include HYSTEM™ C includes GLYCOSIL® and EXTRALINK® but also thiol modified denatured collagen fibris called GELIN-S® to accommodate some cell attachment needs (e.g. Stem cells). HYSTEM™ can also include HYSTEM™ HP (HYSHPO20-1KT, sigmaaldrich.com/catalog/product/sigma/hyshp020?lang=en&region=US) which includes all of HYSTEM™ C plus heparin sulfate to ensure growth factor release at the immediate proximity of cells. In embodiments, the hydrogel comprises more than one HYSTEM™ gel. In embodiments, the hydrogel comprises a HYSTEM™ gel that has been further processed, e.g., has been chemically modified. In embodiments, the hydrogel comprises a HYSTEM gel and an alginate gel, agarose gel, collagen gel and/or a different hyaluronan gel.

In embodiments of the methods provided herein, the population of cells is suspended in the hydrogel. In embodiments, the freezing of the population of cells is performed either in suspension or attached in a container. In embodiments, the freezing of the population of cells is performed with the population of cells suspended in a hydrogel.

In embodiments of the methods provided herein, the concentration of the monosaccharide in the composition is adjusted prior to freezing. As used herein, a concentration prior to freezing is a concentration of a component in the composition prior to the application of the methods provided herein.

In embodiments of the methods provided herein, the concentration of the monosaccharide prior to freezing is from about 0.6 M to about 2.0 M, or from about 0.7 M to about 1.7 M, or from about 0.8 M to about 1.4 M, or from about 0.2 M to about 1.0 M. In embodiments, the concentration of the monosaccharide prior to freezing is from about 0.3 M to about 0.8 M. In embodiments, the concentration of the monosaccharide prior to freezing is from about 0.4 M to about 0.6 M. In embodiments, the concentration of the monosaccharide prior to freezing is from about 0.1 M to about 1.0 M. In embodiments, the concentration of the monosaccharide prior to freezing is from about 0.25 M to about 0.75 M. In embodiments, the concentration of the monosaccharide prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments, a monosaccharide can include both the D and the L forms of the sugar. In some embodiments the monosaccharide is glucose, fructose, galactose, mannose, ribose or deoxyribose. In embodiments, the saccharides can include non-naturally occurring or semi-artificial monosaccharides. In embodiments, the saccharides can include (i) Hexoses (contain 6 carbons) including: D- and L-allose, D- and L-altrose, D- and L-fucose, D- and L-gulose, D-sorbose, D-tagatose, (ii) Pentoses (contain 5 carbons): D- and L-arabinose, D- and L-lyxose, Rhamnose, D-ribose, Ribulose and its synthetic form sucroribulose, and D-xylose or wood sugar.

In embodiments of the methods provided herein, the monosaccharide is glucose, fructose or galactose. In embodiments, the monosaccharide is glucose.

In embodiments of methods where the monosaccharide is glucose, the concentration of the glucose prior to freezing is from about 0.2 M to about 1.0 M. In embodiments, the concentration of the glucose prior to freezing is from about 0.3 M to about 0.8 M. In embodiments, the concentration of the glucose prior to freezing is from about 0.4 M to about 0.6 M. In embodiments, the concentration of glucose prior to freezing is from about 0.1 M to about 1.0 M. In embodiments, the concentration of glucose prior to freezing is from about 0.25 M to about 0.75 M. In embodiments, the concentration of glucose prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments of the methods provided herein, the concentration of the urea prior to freezing is from about 0.6 M to about 2.0 M, or from about 0.7 M to about 1.7 M, or from about 0.8 M to about 1.4 M, or from about 0.2 M to about 1.0 M. In embodiments, the concentration of the urea prior to freezing is from about 0.3 M to about 0.8 M. In embodiments, the concentration of the urea prior to freezing is from about 0.4 M to about 0.6 M. In embodiments, the concentration of urea prior to freezing is from about 0.1 M to about 1.0 M. In embodiments, the concentration of urea prior to freezing is from about 0.25 M to about 0.75 M. In embodiments, the concentration of urea prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments of the methods provided herein, the molar ratio of urea to monosaccharide can be adjusted by varying the concentrations of either component. In embodiments, the molar ratio of urea to monosaccharide prior to freezing is from about 1:3 to about 3:1. In embodiments, the molar ratio of urea to monosaccharide prior to freezing is from about 1:2 to about 2:1. In embodiments, the molar ratio of urea to monosaccharide prior to freezing is about 1:1. In embodiments, the molar ratio of urea to monosaccharide prior to freezing is from about 1:5 to about 5:1. In embodiments, the molar ratio of urea to monosaccharide prior to freezing is from about 1:4 to about 4:1. In embodiments, the molar ratio of urea to monosaccharide prior to freezing is about 5:1, 4:1, 3:1, 2:1, 1:1, 1:2, 1:3, 1:4 or 1:5.

In embodiments of methods where the monosaccharide is glucose, the molar ratio of urea to glucose prior to freezing is from about 1:3 to about 3:1. In embodiments, the molar ratio of urea to glucose prior to freezing is from about 1:2 to about 2:1. In embodiments, the molar ratio of urea to glucose prior to freezing is about 1:1. In embodiments, the molar ratio of urea to glucose prior to freezing is from about 1:5 to about 5:1. In embodiments, the molar ratio of urea to glucose prior to freezing is from about 1:4 to about 4:1. In embodiments, the molar ratio of urea to glucose prior to freezing is about 5:1, 4:1, 3:1, 2:1, 1:1, 1:2, 1:3, 1:4 or 1:5.

In embodiments of the methods provided herein, the freezing can be performed with the cells in suspension or attached to a surface of a container. In embodiments, the freezing can be performed with some cells in suspension and some cells attached to a surface of a container, e.g., with both suspended and attached cells. In embodiments, the freezing is performed with the population of cells suspended in a hydrogel. Suitable hydrogels are described herein.

In embodiments, the freezing is performed in a container with or without collagen coating. In embodiments, the container is a glass or plastic container with or without a membrane for cell attachment or growth. In embodiments, the freezing is performed on a surface with or without collagen coating. In embodiments, the surface is glass or plastic.

In embodiments of the methods provided herein, the population of cells is from about 1×10⁴ cells per mL to 1×10⁷ cells per mL. In embodiments, the population of cells is from about 1×10⁵ cells per mL to 4×10⁵ cells per mL. In embodiments, the population of cells is from about 2×10⁵ cells per mL to 2.5×10⁵ cells per mL. In embodiments, the population of cells is about 1×10⁵ cells per mL, 1.5×10⁵ cells per mL, 2×10⁵ cells per mL, 2.5×10⁵ cells per mL, 3×10⁵ cells per mL, 3.5×10⁵ cells per mL, 4×10⁵ cells per mL, 4.5×10⁵ cells per mL or 5×10⁵ cells per mL.

In embodiments of the methods provided herein, the bulking agent comprises a disaccharide. In embodiments, the concentration of disaccharide prior to freezing is from about 0.1 M to about 1.0 M. In embodiments, the concentration of disaccharide prior to freezing is from about 0.1 M to about 0.5 M. In embodiments, the concentration of disaccharide prior to freezing is from about 0.2 M to about 0.4 M. In embodiments, the concentration of disaccharide prior to freezing is from about 0.1 M to about 0.4 M. In embodiments, the concentration of disaccharide prior to freezing is from about 0.1 M to about 0.3 M. In embodiments, the concentration of disaccharide prior to freezing is from about 0.25 M to about 0.45 M. In embodiments, the concentration of disaccharide prior to freezing is from about 0.25 M to about 0.75 M. In embodiments, the concentration of disaccharide prior to freezing is from about 0.3 M to about 0.8 M. In embodiments, the concentration of disaccharide prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments, the disaccharide is sucrose, lactose, maltose, trehalose, cellobiose, chitobiose, lactulose, isomaltose melibiose or gentiobiose. In embodiments, the disaccharide has a C₁-C₁ glycosidic linkage. In embodiments, embodiments, the disaccharide can include, e.g., an α(1→2)β, β(1→4) or α(1→4) linkage. In embodiments, the disaccharide is sucrose.

In embodiments of methods where sucrose is used, the concentration of sucrose prior to freezing is from about 0.1 M to about 1.0 M. In embodiments, the concentration of sucrose prior to freezing is from about 0.1 M to about 0.5 M. In embodiments, the concentration of sucrose prior to freezing is from about 0.2 M to about 0.4 M. In embodiments, the concentration of sucrose prior to freezing is from about 0.1 M to about 0.4 M. In embodiments, the concentration of sucrose prior to freezing is from about 0.1 M to about 0.3 M. In embodiments, the concentration of sucrose prior to freezing is from about 0.25 M to about 0.45 M. In embodiments, the concentration of sucrose prior to freezing is from about 0.25 M to about 0.75 M. In embodiments, the concentration of sucrose prior to freezing is from about 0.3 M to about 0.8 M. In embodiments, the concentration of sucrose prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments of the methods provided herein, the bulking agent comprises a sugar alcohol. In embodiments, the concentration of sugar alcohol prior to freezing is from about 0.1 M to about 1.0 M. In embodiments, the concentration of sugar alcohol prior to freezing is from about 0.3 M to about 0.8 M. In embodiments, the concentration of sugar alcohol prior to freezing is from about 0.4 M to about 0.6 M. In embodiments, the concentration of sugar alcohol prior to freezing is from about 0.1 M to about 1.0 M. In embodiments, the concentration of sugar alcohol prior to freezing is from about 0.25 M to about 0.75 M. In embodiments, the concentration of sugar alcohol prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments, the sugar alcohol is mannitol, sorbitol, galactitol, fucitol, iditol or inositol. In embodiments, the sugar alcohol is mannitol. In embodiments, the concentration of mannitol prior to freezing is 7 from about 0.1 M to about 1.0 M. In embodiments, the concentration of mannitol prior to freezing is from about 0.3 M to about 0.8 M. In embodiments, the concentration of mannitol prior to freezing is from about 0.4 M to about 0.6 M. In embodiments, the concentration of mannitol prior to freezing is from about 0.1 M to about 1.0 M. In embodiments, the concentration of mannitol prior to freezing is from about 0.25 M to about 0.75 M. In embodiments, the concentration of mannitol prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments of the methods provided herein, the bulking agent comprises both a disaccharide and a sugar alcohol. In embodiments, the bulking agent comprises only a disaccharide. In embodiments, the bulking agent comprises only a sugar alcohol. In embodiments, the bulking agent comprises both sucrose and mannitol. In embodiments, the bulking agent comprises only sucrose. In embodiments, the bulking agent comprises only mannitol.

In embodiments, the methods provided herein do not use DMSO for preservation of cells. In embodiments, the methods provided herein allow for use of DMSO at a lower concentration than would be needed for preservation of cells using traditional methods. In embodiments of the methods provided herein, the composition does not comprise DMSO. In embodiments, the composition comprises DMSO at a concentration of from about 1% to about 10% prior to freezing. In embodiments, the composition comprises DMSO at a concentration of from about 1 to about 10% prior to freezing, or from about 1.5% to about 8%, or from about 2% to about 5%. In embodiments, the composition comprises DMSO at a concentration of from about 3% to about 8% prior to freezing. In embodiments, the composition comprises DMSO at a concentration of from about 1% to about 5% prior to freezing. In embodiments, the composition comprises DMSO at a concentration of from about 1% to about 4.5% or of from about 1% to about 4% or of from about 1% to about 3% prior to freezing. In embodiments, the composition comprises DMSO at a concentration of about 1%, 2%, 3%, 4%, 5%, 6%, 7%, 8%, 9% or 10% prior to freezing.

In embodiments of the methods provided herein, prior to the freezing in (a), the population of cells is isolated and contacted with a poration solution. The term “poration solution” as used herein relates to a solution that is typically comprised of cell medium and sugar. It does not have the purpose to perforate the cell membrane. Rather, it is used as a pre-incubation solution with the purpose to introduce sugar, e.g., trehalose, which is contained therein, into the cells, which then facilitates cryoprotection. In embodiments, the methods comprise a step wherein prior to freezing, the population of cells is isolated (e.g., centrifuged or filtered or by simply removal of medium from the cells, which usually are attached to the wall or bottom of the container, such as a culture flask) and resuspended in a poration solution. The poration solution comprises a sugar, e.g., trehalose in an aqueous solution. In embodiments, the poration solution comprises sugar and water. In embodiments, the poration solution comprises trehalose and water. In embodiments, the poration solution comprises trehalose and cell culture medium. In embodiments, the population of cells is placed in the poration solution, and then subjected to thermal treatment. In embodiments, the thermal treatment is performed as outlined by He et al., (He, X., et al., Cell Preservation Technology 4, 178-187 (2006)). In embodiments, the thermal treatment includes, e.g., placing the population of cells in the poration solution, then cooling the composition for 1 to 60 minutes, e.g., 2 to 50 minutes, 3 to 40 minutes, 4 to 30 minutes, 5 to 20 minutes, 5 to 15 minutes, or about 10 minutes, followed by heating the composition for 1 to 60 minutes, e.g., 2 to 50 minutes, 3 to 40 minutes, 4 to 30 minutes, 5 to 20 minutes, 5 to 15 minutes. The cooling and heating can be repeated one, two, three, four, five, six, seven, eight, nine or ten times. In embodiments, the cooling and heating can be repeated one, two, or three times. In embodiments, the cooling was from −20° C. to a 10° C., or −10° C. to 10° C., or 0° C. to 10° C., or 2° C. to 8° C. In embodiments, the warming was from 0° C. to 50° C., or 4° C. to 40° C., 8° C. to 40° C. or 20° C. to 40° C. In embodiments, the difference between the cooling temperature and the warming temperature was greater than 4° C., greater than 6° C., greater than 8° C., greater than 10° C., greater than 15° C., or greater than 20° C.

In embodiments where the poration solution comprises trehalose, the concentration of trehalose in the poration solution is from about 0.1 M to about 1.0 M. In embodiments, the concentration of trehalose in the poration solution is from about 0.2 M to about 0.6 M. In embodiments, the concentration of trehalose in the poration solution is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M or 1.0 M.

In embodiments, the tonicity of the composition can affect cell viability and attachment. “Tonicity” refers to the ability of an extracellular solution to make water move into or out of a cell by osmosis. Tonicity is related to a solution's osmolarity, i.e., the total concentration of all solutes in the solution. An “isotonic” solution refers to a solution that is the same osmolarity as the cell, and thus there is no net movement of water into or out of the cell. A “hypertonic” solution refers to a solution that is higher than the osmolarity of the cell, and thus water will move out of the cell into the extracellular solution. A “hypotonic” solution refers to a solution that is lower in osmolarity of the cell, and thus water will move into the cell from the extracellular solution. In embodiments, the composition comprising the population of cells is an isotonic solution. In embodiments, the composition comprising the cells is a hypertonic solution. In some embodiments, the tonicity of the composition facilitates the lyophilization process, protects the cells from damage or lysis, and/or increases cell viability.

In some embodiments, the aqueous component of the composition comprises buffer. Non-limiting examples of buffers include phosphate buffer, Tris buffer, HEPES buffer, acetate buffer, bicarbonate buffer, citrate buffer, tricine buffer, TES buffer, and the like. In embodiments, the aqueous component of the composition comprises phosphate buffer, Tris buffer, acetate buffer, bicarbonate buffer, histidine buffer, citrate buffer, or combinations thereof.

In some embodiments, the aqueous component of the composition comprises cell culture medium. Non-limiting examples of cell culture medium include DMEM, MEM, RPMI 1640, EXPI293, OPTI-MEM, and STEMPRO from GIBCO, HYQ-RS from HYCLONE, X-VIVO and Hybridoma Serum-Free Culture Media from LONZA BIOWHITTAKER, and the like. In embodiments, the medium is free of serum. In embodiments, the medium is free of fetal bovine serum, bovine serum, or human serum. In embodiments, the medium is a chemically defined medium. An exemplary chemically-defined medium may include a basal media (such as, e.g., DMEM, F12, or RPMI 1640), sugars such as dextrose and glucose, amino acids, vitamins, inorganic salts, buffers, antioxidants, growth factors and energy sources, recombinant serum albumin, chemically defined lipids, recombinant insulin and/or zinc, recombinant transferrin or iron, selenium, and/or antioxidant thiols such as 2-mercaptoethanol or 1-thioglycerol. In embodiments, the cell culture medium comprises dextrose, glucose, amino acids, recombinant serum albumin, growth factors, or combinations thereof.

In embodiments of the methods provided herein the cells are reconstituted in a reconstitution agent, e.g., any liquid that can be used to suspend cells that have been frozen or lyophilized. In embodiments, wherein the reconstitution agent comprises cell culture medium. In embodiments, wherein the reconstitution agent comprises cell culture medium as described above.

In some embodiments, the lyophilization process does not lyse or damage the cells, at least not all cells. In embodiments, the lyophilized cells are capable of being reconstituted and being viable. In the context of lyophilization, “reconstitution” refers to the process of rehydrating a lyophilized substance, e.g., lyophilized cells. In embodiments, cells that are reconstituted after lyophilization are viable. In embodiments, cells that are reconstituted after lyophilization are capable of performing the same cellular functions as a cell not subjected to lyophilization. In embodiments, cells that are reconstituted after lyophilization are viable. In embodiments, cells that are reconstituted after lyophilization retain the ability to adhere or attach to other cells. In embodiments, cells that are reconstituted after lyophilization retain the ability to proliferate. In embodiments, stem cells that are reconstituted after lyophilization retain the ability to differentiate. In embodiments, cells that are reconstituted after lyophilization are suitable for use in cell therapy, e.g., introduction into a human or animal. In embodiments. cells that are reconstituted after lyophilization are attaching to the culture flask, proliferating, and are of regular morphology, thus indicating viability and, therefore, regular cell function.

In some embodiments, the reconstitution of lyophilized cells comprises resuspending the lyophilized cells in a reconstitution agent to form a reconstituted composition. In the context of lyophilization, “reconstitution agent” refers to a substance that resuspends and rehydrates the lyophilized product. In embodiments, the reconstitution agent comprises water. In embodiments, the reconstitution agent comprises buffer. In embodiments, the reconstitution agent comprises cell culture media. In embodiments, the reconstitution agent comprises polyvinylpyrrolidone (PVP). In embodiments, the reconstitution agent comprises trehalose. In embodiments, the reconstitution agent comprises PVP, trehalose, or combinations thereof. In embodiments, the reconstitution agent comprises phosphate buffer solution (PBS). In some embodiments, the reconstitution agent can be a solution known to maintain the viability of the cells.

The methods provided herein may be used for the freezing or lyophilization or cells of various types and from various sources. In some embodiments, the cell is obtained from cell culture, i.e., cultured cells. In embodiments, the population of cells comprises mammalian cells. In embodiments, the population of cells comprises stem cells. In embodiments, the population of cells comprises pluripotent stem cells, embryonic stem cells, mesenchymal stem cells, or hematopoietic stem cells. In embodiments, the population of cells comprises mesenchymal stem cells. In embodiments, the population of cells comprises induced pluripotent stem cells. In embodiments, the population of cells comprises neuroblastoma cells. In embodiments, the neuroblastoma cells are SK-N-AS cells. In embodiments, any of the above listed cells are human cells.

In embodiments, the population of cells comprises bacterial cells. Examples of such bacterial cells include, but are not limited to, E. coli, S. aureus, V cholerae, S. pneumoniae, B. subtilis, C. crescentus, M. genitalium, A. fischeri, Synechocystis, P. fluorescens, A. vinelandii, S. coelicolor. In embodiments, the bacterial cell is of bacteria used in preparation of food and/or beverages. Non-limiting exemplary genera of such cells include, but are not limited to, Acetobacter, Arthrobacter, Bacillus, Bifidobacterium, Brachybacterium, Brevibacterium, Carnobacterium, Corynebacterium, Enterococcus, Gluconacetobacter, Hafnia, Halomonas, Kocuria, Lactobacillus (including L. acetotolerans, L. acidipiscis, L. acidophilus, L. alimentarius, L. brevis, L. bucheri, L. casei, L. curvatus, L. fermentum, L. hilgardii, L. jensenii, L. kimchii, L. lactis, L. paracasei, L. plantarum, and L. sakei), Leuconostoc, Microbacterium, Pediococcus, Propionibacterium, Weissella, and Zymomonas.

In embodiments, the population of cells comprises eukaryotic cells. In embodiments, the eukaryotic cells are animal or human cells. In embodiments, the eukaryotic cells are a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. In embodiments, the eukaryotic cells are CHO-cell lines. In embodiments, the eukaryotic cells are CHO cells. In embodiments, the cells are CHO-K1 cells, CHO-K1 SV cells, DG44 CHO cells, DUXB11 CHO cells, CHOS cells, CHO GS knock-out cells, CHO FUT8 GS knock-out cells, CHOZN cells, or CHO-derived cells. The CHO GS knock-out cells (e.g., GSKO cells) are, for example, CHO-K1 SV GS knockout cells. Eukaryotic cells can also be avian cells, cell lines or cell strains, such as, for example, EBX cells, EB14, EB24, EB26, EB66, or Ebvl3.

In some embodiments, the eukaryotic cells are a human cells. In embodiments, the human cell are stem cells. In embodiments, the methods provided herein are advantageous for producing lyophilized stem cells that are viable upon reconstitution, e.g., for use in cell therapy. The stem cells can be, for example, pluripotent stem cells, including embryonic stem cells (ESCs), adult stem cells, induced pluripotent stem cells (iPSCs), tissue specific stem cells (e.g., hematopoietic stem cells, neural stem cells, epithelial stem cells, skin stem cells, and the like) and mesenchymal stem cells (MSCs). In embodiments, the stem cell is a mesenchymal stem cell (MSC). In embodiments, the MSC is obtained from the bone marrow, cord blood, peripheral blood, fallopian tube, liver, and/or lung of a human subject. In embodiments, the stem cell is an induced pluripotent stem cell (iPSC). In embodiments, the iPSC has at least one vector capable of expressing one or more pluripotency factors. In embodiments, the iPSC is derived from a fibroblast, keratinocyte, peripheral blood mononuclear cell (PBMCs), hepatocytes, neuronal cell, B cell, muscle cell, adrenal cell, and/or renal epithelial cell of a human subject, or any other type of cell known to be suitable for becoming an induced pluripotent stem cell. In embodiments, the human cell is a differentiated form of any of the cells described herein. In embodiments, the cell comprises at least one vector capable of expressing one or more pluripotency factors, but the pluripotency factors have not yet been expressed before lyophilization, thus the cell has not been induced to pluripotency yet. In embodiments, the eukaryotic cell is a cell derived from any primary cell in culture.

In some embodiments, the eukaryotic cells are hepatocytes such as a human hepatocytes, animal hepatocytes, or a non-parenchymal cells. For example, the eukaryotic cells can be plateable metabolism qualified human hepatocytes, plateable induction qualified human hepatocytes, plateable human hepatocytes, suspension qualified human hepatocytes (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). In embodiments, the cells are from the eye. In embodiments, the cells are retinal cells, scleral cells, choroidal epithelial cells, macrophage cells, or immune cells.

In some embodiments, the cells are obtained from a cell line. Non-limiting examples of cell lines include MOLT-4 (differentiated or undifferentiated), Jurkat, HL60 (differentiated or undifferentiated), U-937 (differentiated or undifferentiated), HDLM-2, THP-1 (differentiated or undifferentiated), GA10, Ramos, HUVEC, PANC-1, Expi293, HaCat, HCT-15, H-2228, peripheral blood mononuclear cells (PBMCs), KU-812, MC-04, HT-1376, TT, HCT-1116, MCF-7, Calu-3, and the like. Exemplary monocytic cell lines include THP-1, differentiated THP-1, HL60, and differentiated HL60. An exemplary NK cell line is NK92. Exemplary T cell lines include Jurkat and Molt-4. An exemplary B cell line is GA-10. Exemplary endothelial cell lines include HUVEC and differentiated HUVEC. Exemplary hepatocytic cell lines include HepG2 and differentiated HepG2. Exemplary epithelial cell lines include A549, A431, Caco-2, HT29, LNCap, SKOV3, SW480, PC3, MDMB-468, MDMB-231, MCF7, HT-1376, PANC-1, HCT15, Calu-3, Skov3, Bewo, K562, and HeLa. Further additional cell lines include, e.g., HT-29 sARPE-19, SH-SY5Y, and U87-MG.

Further examples of cells include T cells, B cells, dendritic cells, NK cells, monocytes, macrophages, granulocytes, platelets, erythrocytes, endothelial cells (e.g., an aortic endothelial cells), epithelial cells, stem cell precursor cells, mesenchymal stem cells, hematopoietic stem cells, leukocytes, senescent cells, adipose cells, hepatocytes, myocytes, or skeletal muscle cells. T cells include, e.g., helper T cells, such as the subtypes Th1, Th2, Th9, Th17, Th22, and Tfh; regulatory T cells; killer T cells; γδ TCR+ T cells; and natural killer T cells. Adipose cells include, e.g., normal adipocytes, diabetic adipocytes, omental adipocytes, MSC-derived adipocytes, preadipocytes, and omental preadipocytes.

In some embodiments, the eukaryotic cells are plant cells. For example, the plant cell can be of a crop plant such as cassava, corn, sorghum, wheat, or rice. The plant cells can be of an algae, tree, or vegetable. The plant cells can be of a monocot or dicot or of a crop or grain plant, a production plant, fruit, or vegetable. For example, the plant cells can be of a tree, e.g., a citrus tree such as orange, grapefruit, or lemon tree; peach or nectarine trees; apple or pear trees; nut trees such as almond or walnut or pistachio trees; nightshade plants, e.g., potatoes; plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the cells include cells that are in a microbiota. In embodiments, the cells are a combination of cells that comprise a microbiota. In embodiments, the cells comprise a full array of microorganisms in the microbiota that live on and/or in an organism, e.g., humans, a domesticated animal (e.g., a cow, pig, chicken, horse, etc.), or a zoo animal. In embodiments, the microbiota includes bacteria, archaea (primitive single-celled organisms), fungi, and even some protozoans and nonliving viruses. In embodiments, the microbiota is a gastrointestinal microbiota (e.g., a esophageal, stomach, and/or intestinal microbiota), an oral microbiota, a urinary tract microbiota, a nasal microbiota, respiratory microbiota, a skin microbiota, a vaginal microbiota, a rectal microbiota, or a combination thereof. In embodiments, the microbiota is an infant's microbiota. In embodiments, the microbiota is an adult's microbiota.

In the lyophilization methods provided herein, the removing of water comprises lowering pressure, applying heat, or both, to the frozen composition to remove water. Lyophilization methods are described herein. In embodiments, the removing water comprises a primary drying step and a secondary drying step. Primary and secondary drying steps are described herein. In embodiments, the removing water comprises only a primary drying step. In embodiments, the primary drying step comprises lowering pressure to remove the aqueous component. In embodiments, the secondary drying step comprises applying heat to remove the aqueous component.

In some embodiments, the freezing occurs at between about −30° C. to about −100° C. In embodiments, the freezing occurs at between about −60° C. to about −90° C. In embodiments, the freezing occurs at between about −70° C. to about −80° C. In embodiments, the freezing occurs at about −60° C., about −65° C., about −70° C., about −75° C., about −80° C., about −85° C., or about −90° C. In embodiments, the freezing occurs at between about −10° C. to about −100° C. In embodiments, the freezing occurs at between about −20° C. to about −90° C. In embodiments, the freezing occurs at between about −40° C. to about −60° C. In embodiments, the freezing lowers the temperature of the composition to about −50° C. In embodiments, the freezing lowers the temperature of the composition to about −80° C. In embodiments, the freezing lowers the temperature of the composition to about −40° C., −50° C., −60° C., −70° C., −80° C. or −90° C.

In some embodiments, the freezing lowers the temperature of the composition to about −50° C., water is removed at a pressure of a chamber pressure of about 20 mTorr to about 40 mTorr. In embodiments, the freezing lowers the temperature of the composition to about −40° C., water is removed at a pressure of a chamber pressure of about 60 mTorr to about 80 mTorr.

Once treated using the methods provided herein, the cells can be stored for extended periods. In embodiments of the methods of freezing cells, the resuspending the frozen cells occurs greater than two hours after freezing. In embodiments, the resuspending the frozen cells occurs greater than one day after freezing. In embodiments, the resuspending the frozen cells occurs greater than one week after freezing. In embodiments, the resuspending the frozen cells occurs greater than one month after freezing.

In embodiments of the methods of lyophilizing cells, the resuspending the lyophilized cells occurs greater than two hours after the removing the water. In embodiments, the resuspending the lyophilized cells occurs greater than one day after the removing the water. In embodiments, the resuspending the lyophilized cells occurs greater than one week after the removing the water. In embodiments, the resuspending the lyophilized cells occurs greater than one month after the removing water.

In embodiments of the methods, the frozen or lyophilized cells are stored below about −20° C. prior to the resuspending. In embodiments, the frozen or lyophilized cells are stored at about −20° C. to about 30° C. prior to the resuspending. In embodiments, the frozen or lyophilized cells are stored at about 4° C. to about 28° C. prior to the resuspending. In embodiments, the frozen or lyophilized cells are stored at about 10° C. to about 27° C. prior to the resuspending. In embodiments, the frozen or lyophilized cells are stored at about 2° C. to about 8° C. prior to the resuspending.

In embodiments, the frozen or lyophilized cells are stored at less than about −20° C. for greater than 2 days prior to the resuspending, e.g., greater than 2 weeks, 3 weeks, 1 month, 2 months, 3 months or 6 months prior to resuspending. In embodiments, the frozen or lyophilized cells are stored at greater than about −20° C. for greater than 2 days prior to the resuspending, e.g., greater than 2 weeks, 3 weeks, 1 month, 2 months, 3 months or 6 months prior to resuspending. In embodiments, the lyophilized cells are stored at about 20° C. to about 25° C. for greater than 1 week prior to the resuspending, e.g., greater than 2 weeks, 3 weeks, 1 month, 2 months, 3 months or 6 months prior to resuspending. In embodiments, the lyophilized cells are stored at about 2° C. to about 8° C. for greater than 1 week prior to the resuspending, e.g., greater than 2 weeks, 3 weeks, 1 month, 2 months, 3 months or 6 months prior to resuspending.

As discussed herein, the methods provide viable cells following freezing or lyophilization. A “viable” cell refers to an alive and functioning cell, e.g., a cell that is capable of survival and performing normal cellular functions. In embodiments, a normal cellular function of a cell is adhesion or attachment to other cells. “Cell adhesion” or “cell attachment” refers to a process by which cells interact and attach to neighboring cells and/or to the surface of their growth containers through interactions of surface proteins. Cells not capable of attaching may be unable to proliferate or perform normal cellular functions. For example, a stem cell that is not capable of attaching may be unable to differentiate. In another example, a cell that is introduced into a patient for cell therapy, but is unable to attach, may not provide therapeutic benefits because the cell is not capable of performing normal functions in the patient. Thus, in embodiments, a viable cell is a cell that is capable of attachment. In embodiments, a viable cell is a cell that is capable of proliferation. In embodiments, a viable stem cell is a stem cell that is capable of differentiation.

In some embodiments, viability of a cell is determined, e.g., by measuring the redox potential and/or metabolic activity of the cell population. The redox potential and/or metabolic activity of a cell population can be measured by reagents such as resazurin, a component of the reagents ALAMARBLUE® and PRESTOBLUE®; MTT (3-(4,5-dimethylthiazol-2-yl)-2,5-diphenyl tetrazolium bromide), used in the VYBRANT MTT Cell Viability Assay; or XTT (2,3-bis-(2-methoxy-4-nitro-5-sulfophenyl)-2H-tetrazolium-5-carboxanilide), used in the CYQUANT XTT Cell Viability Assay. In embodiments, viability of a cell is determined by measuring or inspecting the integrity of the cellular membrane, for example, using microscopy or flow cytometry. Microscopy, when used for the determination of viability, allows for example observation if the morphology of the cells looks similar to cells which are known to be viable, and/or whether the cells proliferate and/or whether the cells attached to the wall or to bottom of a culture flask. In embodiments, viability of a cell is measured after a cell is frozen, then thawed or resuspended. In embodiments, viability of a cell is measured after a cell is lyophilized, then reconstituted.

Additional examples of cell viability assays are described in, e.g., Riss et al., “Cell Viability Assays,” 2013 May 1 [Updated 2016 Jul. 1]. In: Sittampalam G S, Coussens N P, Brimacombe K, et al., editors. Assay Guidance Manual [Internet]. Bethesda (MD): Eli Lilly & Company and the National Center for Advancing Translational Sciences; 2004. Available from: www.ncbi.nlm.nih.gov/books/NBK144065.

The skilled artisan can appreciate that when discussing viability of cells, i.e., a population of cells, that not 100% of the cells will be viable. In embodiments, a viable cell population comprises at least 1% viable cells. In embodiments, a viable cell population comprises at least 5% viable cells. In embodiments, a viable cell population comprises at least 10% viable cells. In embodiments, a viable cell population comprises at least 15% viable cells. In embodiments, a viable cell population comprises at least 20% viable cells. In embodiments, a viable cell population comprises at least 30% viable cells. In embodiments, a viable cell population comprises at least 40% viable cells. In embodiments, a viable cell population comprises at least 50% viable cells. In embodiments, a viable cell population comprises at least 60% viable cells. In embodiments, a viable cell population comprises at least 70% viable cells. In embodiments, a viable cell population comprises at least 80% viable cells. In embodiments, a viable cell population comprises at least 90% viable cells. In embodiments, a viable cell population comprises about 1% viable cells to about 99% viable cells, 5% viable cells to about 99% viable cells, 10% viable cells to about 99% viable cells, 20% viable cells to about 99% viable cells, about 30% viable cells to about 99% viable cells, about 40% viable cells to about 99% viable cells, about 50% viable cells to about 99% viable cells, about 60% viable cells to about 99% viable cells, about 70% viable cells to about 99% viable cells, about 80% viable cells to about 99% viable cells, or about 90% viable cells to about 99% viable cells.

In some embodiments, a viable cell population comprises at least 1% of cells capable of attachment. In embodiments, a viable cell population comprises at least 5% of cells capable of attachment. In embodiments, a viable cell population comprises at least 10% of cells capable of attachment. In embodiments, a viable cell population comprises at least 20% of cells capable of attachment. In embodiments, a viable cell population comprises at least 30% of cells capable of attachment. In embodiments, a viable cell population comprises at least 40% of cells capable of attachment. In embodiments, a viable cell population comprises at least 50% of cells capable of attachment. In embodiments, a viable cell population comprises at least 60% of cells capable of attachment. In embodiments, a viable cell population comprises at least 70% of cells capable of attachment. In embodiments, a viable cell population comprises at least 80% of cells capable of attachment. In embodiments, a viable cell population comprises at least 90% of cells capable of attachment. In embodiments, a viable cell population comprises about 1% to about 99% cells capable of attachment, 5% to about 99% cells capable of attachment, 10% to about 99% cells capable of attachment, 20% to about 99% cells capable of attachment, about 30% to about 99% cells capable of attachment, about 40% to about 99% cells capable of attachment, about 50% to about 99% cells capable of attachment, about 60% to about 99% cells capable of attachment, about 70% to about 99% cells capable of attachment, about 80% to about 99% cells capable of attachment, or about 90% to about 99% cells capable of attachment.

In some embodiments, the reconstituted cells are viable. Methods of measuring viability of cells are described herein. In embodiments, cell viability is measured by staining and detecting an intact cell membrane. In embodiments, cell viability is measured by assessing metabolic activity, e.g., by using the ALAMARBLUE®, MTT, or XTT tests. In embodiments, at least 1% of the cells in the reconstituted composition are viable. In embodiments, at least 5% of the cells in the reconstituted composition are viable. In embodiments, at least 10% of the cells in the reconstituted composition are viable. In embodiments, at least 20% of the cells in the reconstituted composition are viable. In embodiments, at least 30% of the cells in the reconstituted composition are viable. In embodiments, at least 40% of the cells in the reconstituted composition are viable. In embodiments, at least 50% of the cells in the reconstituted composition are viable. In embodiments, at least 60% of the cells in the reconstituted composition are viable. In embodiments, at least 70% of the cells in the reconstituted composition are viable. In embodiments, at least 80% of the cells in the reconstituted composition are viable. In embodiments, at least 90% of the cells in the reconstituted composition are viable.

In some embodiments, the reconstituted cells are capable of attachment, as measured by ALAMARBLUE® test. As described herein, the ALAMARBLUE® test assesses metabolic activity of the cells, which can be an indicator of cell attachment. In embodiments, at least 20% of the cells in the reconstituted composition attach as measured by ALAMARBLUE® test. In embodiments, at least 30% of the cells in the reconstituted composition attach as measured by ALAMARBLUE® test. In embodiments, at least 40% of the cells in the reconstituted composition attach as measured by ALAMARBLUE® test. In embodiments, at least 50% of the cells in the reconstituted composition attach as measured by ALAMARBLUE® test. In embodiments, at least 60% of the cells in the reconstituted composition attach as measured by ALAMARBLUE® test. In embodiments, at least 70% of the cells in the reconstituted composition attach as measured by ALAMARBLUE® test. In embodiments, at least 80% of the cells in the reconstituted composition attach as measured by ALAMARBLUE® test. In embodiments, at least 90% of the cells in the reconstituted composition attach as measured by ALAMARBLUE® test.

### Compositions Comprising a Population of Viable Cells

In embodiments, the present disclosure provides a composition comprising a population of viable cells, an aqueous component, urea and a monosaccharide. In embodiments, the composition further comprises a bulking agent. In embodiments, the composition further comprises a hyaluronan gel.

In embodiments of the compositions, the concentration of the monosaccharide is from about 0.2 M to about 1.25 M, or from about 0.3 M to about 0.8 M, or from about 0.4 M to about 0.6 M.

In embodiments of the compositions, the concentration of the urea is from about 0.2 M to about 1.25 M, or from about 0.2 M to about 0.8 M, or from about 0.4 M to about 0.6 M.

In embodiments of the compositions, the concentration of the monosaccharide is from about 0.6 M to about 2.0 M, or from about 0.7 M to about 1.7 M, or from about 0.8 M to about 1.4 M.

In embodiments of the compositions, the concentration of the urea is from about 0.6 M to about 2.0 M, or from about 0.7 M to about 1.7 M, or from about 0.8 M to about 1.4 M.

In embodiments of the compositions, the molar ratio of the urea to monosaccharide in the composition is from about 5:1 to about 1:5, or from about 1:3 to about 3:1, or from about 1:2 to about 2:1, or is about 1:1.

In embodiments of the compositions, the bulking agent comprises a) a disaccharide, wherein the disaccharide is sucrose, lactose, maltose, trehalose, cellobiose, chitobiose, lactulose, isomaltose melibiose or gentiobiose; or b) a sugar alcohol, wherein the sugar alcohol is mannitol, sorbitol, galactitol, fucitol, iditol or inositol; or c) both a disaccharide and a sugar alcohol; or d) both sucrose and mannitol. In embodiments of the compositions, the concentration of disaccharide prior to freezing is from about 0.1 M to about 1.0 M, or from about 0.1 M to about 0.5 M, or from about 0.2 M to about 0.4 M; and/or the concentration of sugar alcohol prior to freezing is from about 0.1 M to about 1.0 M, or from about 0.3 M to about 0.8 M, or from about 0.4 M to about 0.6 M.

In embodiments of the compositions, the composition comprises from about 0.2 M to about 1.25 M urea and from about 0.2 M to about 1.25 M glucose.

In embodiments of the compositions, the composition does not comprise DMSO, or, the composition comprises DMSO at a concentration of from about 1% to about 10% prior to freezing, or the composition comprises DMSO at a concentration of from about 1% to about 5% prior to freezing.

In embodiments, the composition is in a frozen state at a temperature of between about −10° C. to about −100° C., or between about −20° C. to about −90° C., or between about −40° C. to about −60° C. In embodiments, the composition is a lyophilized composition. In embodiments, the composition contains less than about 90% (vol/vol) of water. In embodiments, the composition contains less than about 80% (vol/vol) of water. In embodiments, the composition contains less than about 70% (vol/vol) of water. In embodiments, the composition contains less than about 60% (vol/vol) of water. In embodiments, the composition contains less than about 50% (vol/vol) of water. In embodiments, the composition contains less than about 40% (vol/vol) of water. In embodiments, the composition contains less than about 30% (vol/vol) of water. In embodiments, the composition contains less than about 20% (vol/vol) of water. In embodiments, the composition contains less than about 10%, less than about 9%, less than about 8%, less than about 7%, less than about 6%, less than about 5%, less than about 4%, less than about 3%, less than about 2%, or less than about 1% (vol/vol) of water.

In embodiments, the composition is in a frozen state at a temperature of between about −10° C. to about −100° C., or between about −20° C. to about −90° C., or between about −40° C. to about −60° C.; in embodiments, said composition is a lyophilized composition; in embodiments, said composition contains less than about 90%, or less than about 80%, or less than about 7000, or less than about 60%, or less than about 50%, or less than about 40%, or less than about 300%, or less than about 20%, or less than about 10%, or less than about 9%, or less than about 8%, or less than about 7%, or less than about 6%, or less than about 5%, or less than about 4%, or less than about 3%, or less than about 2%, or less than about 1% (vol/vol) of water.

In embodiments, the present disclosure provides a composition comprising a population of viable cells, an aqueous component, urea, a monosaccharide, optionally a bulking agent, wherein said composition contains less than about 90%, or less than about 80%, or less than about 70%, or less than about 60%, or less than about 50%, or less than about 40%, or less than about 30%, or less than about 20%, or less than about 10%, or less than about 9%, or less than about 8%, or less than about 7%, or less than about 6%, or less than about 5%, or less than about 4%, or less than about 3%, or less than about 2%, or less than about 1% (vol/vol) of water.

In embodiments, the present disclosure provides a composition comprising a population of viable cells, an aqueous component, urea, a monosaccharide, and a bulking agent, wherein the composition contains less than about 10% (vol/vol) water. In embodiments, the composition contains less than about 5% (vol/vol) water. In embodiments, the composition contains less than about 3% (vol/vol) water. In embodiments, the composition contains less than about 1% (vol/vol) water.

In embodiments of the composition, the molar ratio (i.e. the (M/M) ratio) of glucose to urea in the composition is from about 30:1 to about 1:3. In embodiments, the molar ratio of glucose to urea in the composition is from about 15:1 to about 1:2. In embodiments, the molar ratio of glucose to urea in the composition is from about 3:1 to about 1:1. In embodiments, the molar ratio of glucose to urea in the composition is about 30:1, 25:1, 20:1, 15:1, 10:1, 5:1, 3:1, 2:1, 1:1, 1:2, 1:3, 1:4 or 1:5.

In embodiments, the present disclosure also provides a composition comprising a population of viable cells, an aqueous component, urea, a monosaccharide, and optionally a bulking agent, wherein the molar ratio of glucose to urea in the composition is from about 30:1 to about 1:3. In embodiments, the molar ratio of glucose to urea in the composition is from about 15:1 to about 1:2. In embodiments, the molar ratio of glucose to urea in the composition is from about 3:1 to about 1:1. In embodiments, the molar ratio of glucose to urea in the composition is about 30:1, 25:1, 20:1, 15:1, 10:1, 5:1, 3:1, 2:1, 1:1, 1:2, 1:3, 1:4 or 1:5.

In embodiments of the compositions provided herein, monosaccharide is glucose, fructose, galactose, mannose, ribose or deoxyribose. In embodiments, the monosaccharide is glucose, fructose or galactose. In embodiments, the monosaccharide is glucose.

In embodiments, the present disclosure also provides a composition comprising a population of viable cells, an aqueous component, from about 0.2 M to about 1.0 M urea, from about 0.2 M to about 1.0 M glucose, and optionally a bulking agent.

In embodiments, the composition comprises from about 0.3 M to about 0.8 M glucose. In embodiments, the composition comprises from about 0.4 M to about 0.6 M glucose. In embodiments, the composition comprises about 0.5 M glucose.

In embodiments, the composition comprises from about 0.3 M to about 0.8 M urea. In embodiments, the composition comprises from about 0.4 M to about 0.6 M urea. In embodiments, the composition comprises about 0.5 M urea.

In embodiments, the compositions further comprise sucrose. In embodiments, the compositions comprise from about 0.1 M to about 0.5 M sucrose. In embodiments, the compositions comprise from about 0.25 M to about 0.5 M sucrose. In embodiments, the compositions comprise about 0.25 M sucrose. In embodiments, the compositions comprise about 0.5 M sucrose.

In embodiments, the compositions further comprise mannitol. In embodiments, the compositions comprise from about 0.3 M to about 0.8 M mannitol. In embodiments, the compositions comprise from about 0.1 M to about 0.5 M mannitol. In embodiments, the compositions comprise from about 0.25 M to about 0.5 M mannitol. In embodiments, the compositions comprise about 0.25 M mannitol. In embodiments, the compositions comprise about 0.5 M mannitol. In embodiments, the compositions comprise about 0.4 M mannitol.

In embodiments of the compositions, the population of viable cells comprises eukaryotic cells or prokaryotic cells as described herein. In embodiments, the population of viable cells comprises mammalian cells. In embodiments, the population of viable cells comprises stem cells. In embodiments, the population of viable cells comprises pluripotent stem cells, embryonic stem cells, mesenchymal stem cells, or hematopoietic stem cells. In embodiments, the population of viable cells comprises mesenchymal stem cells. In embodiments, the population of viable cells comprises induced pluripotent stem cells. In embodiments, the population of viable cells comprises neuroblastoma cells. In embodiments, the neuroblastoma cells are SK-N-AS cells.

In embodiments, the compositions provided herein do not comprise DMSO. In embodiments, the compositions provided herein comprise DMSO at a lower concentration than would be needed in a composition for preservation of cells using traditional methods. In embodiments, the composition does not comprise DMSO. In embodiments, the composition comprises DMSO at a concentration of from about 1% to about 10%. In embodiments, the composition comprises DMSO at a concentration of from about 2% to about 5%. In embodiments, the composition comprises DMSO at a concentration of from about 3% to about 8%. In embodiments, the composition comprises DMSO at a concentration of from about 1% to about 5%. In embodiments, the composition comprises DMSO at a concentration of from about 1% to about 3%. In embodiments, the composition comprises DMSO at a concentration of about 1%, 2%, 3%, 4%, 5%, 6%, 7%, 8%, 9% or 10%. In embodiments, the compositions comprise DMSO at the above concentrations on a percent volume (% vol.) basis. In embodiments, the compositions comprise DMSO at the above concentrations on a percent weight (% w) basis. In some embodiments, the concentration of DMSO is less than 1%, less than 0.5%, less than 0.2% or less than 0.1% (wt %) of the compositions.

### Additional Methods

In embodiments, the present disclosure is also directed to a method of producing a population of frozen cells, comprising a step (a) of freezing a composition as defined above to produce the population of frozen cells.

In embodiments, the present disclosure provides methods of freezing a population of cells, comprising freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.

In embodiments, the present disclosure provides methods of lyophilizing a population of cells, comprising: a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and b) removing the aqueous component, in particular water, from the frozen composition to produce a population of lyophilized cells, wherein the aqueous component, in particular water, in the population of lyophilized cells is less than about 10% w/w.

In embodiments, the present disclosure is also directed to a method of producing a population of lyophilized cells, comprising a step (a) of freezing a composition as defined herein and a step (b) of removing at least about 10% (vol/vol) of water from the frozen composition to produce the population of lyophilized cells; in embodiments, step (b) comprises removing at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% (vol/vol) of water.

In embodiments, the present disclosure is also directed to a method of producing a population of lyophilized cells, comprising a step (a) of freezing a composition as defined above and a step (b) of removing at least about 10% (vol/vol) of water from the frozen composition to produce the population of lyophilized cells. In embodiments, step (b) comprises removing at least about 20% (vol/vol) of water. In embodiments, step (b) comprises removing at least about 30% (vol/vol) of water. In embodiments, step (b) comprises removing at least about 40% (vol/vol) of water. In embodiments, step (b) comprises removing at least about 50% (vol/vol) of water. In embodiments, step (b) comprises removing at least about 60% (vol/vol) of water. In embodiments, step (b) comprises removing at least about 70% (vol/vol) of water. In embodiments, step (b) comprises removing at least about 80% (vol/vol) of water. In embodiments, step (b) comprises removing at least about 90%, about 91%, about 92%, about 93%, about 94%, about 95%, about 96%, about 97%, about 98%, or about 99% (vol/vol) of water.

In embodiments, the present disclosure provides methods of producing a viable population of cells, comprising: a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and b) resuspending the population of lyophilized cells in a reconstitution agent to form a reconstituted composition, wherein at least about 1% of the cells in the reconstituted composition are viable.

In embodiments, the present disclosure provides methods of producing a viable population of cells, comprising: a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; b) removing the aqueous component, in particular water, from the frozen composition to produce a population of lyophilized cells, wherein the aqueous component, in particular water, in the population of lyophilized cells is less than about 10% w/w; and c) resuspending the population of lyophilized cells in a reconstitution agent to form a reconstituted composition, wherein at least about 1% of the cells in the reconstituted composition are viable.

In embodiments, the present disclosure is also directed to a method of producing a population of reconstituted viable cells, comprising a step (a) of freezing a composition as defined above to produce a population of frozen cells and a step (b) of resuspending the population of frozen cells in a reconstitution agent to form a reconstituted composition, wherein at least about 1% of the cells are viable.

In embodiments, the present disclosure is also directed to a method of producing a population of reconstituted viable cells, comprising a step (a) of freezing a composition as defined above; a step (b) of removing at least about 10% (vol/vol) of water from the frozen composition to produce the population of lyophilized cells, and a step (c) of resuspending the population of lyophilized cells in a reconstitution agent to form a reconstituted composition, wherein at least about 1% of the cells are viable; in embodiments, step (b) comprises removing at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, or at least about 99% (vol/vol) of water.

In embodiments, the present disclosure provides methods of freezing a population of cells, comprising freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.

In embodiments, the present disclosure provides methods of lyophilizing a population of cells, comprising: a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; and b) removing the aqueous component, in particular water, from the frozen composition to produce a population of lyophilized cells, wherein the aqueous component, in particular water, in the population of lyophilized cells is less than about 10% w/w.

In embodiments, the present disclosure provides methods of producing a viable population of cells, comprising: a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; and b) resuspending the population of lyophilized cells in a reconstitution agent to form a reconstituted composition, wherein at least about 1% of the cells in the reconstituted composition are viable.

In embodiments, the present disclosure provides methods of producing a viable population of cells, comprising: a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; b) removing the aqueous component, in particular water, from the frozen composition to produce a population of lyophilized cells, wherein the aqueous component, in particular water, in the population of lyophilized cells is less than about 10% w/w; and c) resuspending the population of lyophilized cells in a reconstitution agent to form a reconstituted composition, wherein at least about 1% of the cells in the reconstituted composition are viable.

In embodiments of any of the above methods, the composition further comprises from about 1% to about 8% DMSO.

In embodiments of any of the above methods, the composition further comprises a hydrogel. In embodiments, the hydrogel is a hyaluronan gel, alginate gel or collagen gel. In embodiments, the population of cells is suspended in the hydrogel.

In embodiments of any of the above methods, the composition is free of DMSO.

In embodiments of any of the above methods, prior to the freezing in step (a), the population of cells is isolated and contacted with a poration solution. In embodiments, the poration solution comprises trehalose. In embodiments, the concentration of trehalose in the poration solution is from about 0.1 M to about 1.0 M, or from about 0.1 M to about 0.6 M.

In embodiments of any of the above methods, at least about 20% of the cells in the reconstituted composition are viable as measured by ALAMARBLUE® test. In embodiments, at least about 30% of the cells in the reconstituted composition are viable as measured by ALAMARBLUE® test. In embodiments, at least about 40% of the cells in the reconstituted composition are viable as measured by ALAMARBLUE® test.

In embodiments of any of the above methods, at least about 50% of the cells in the reconstituted composition are viable as measured by ALAMARBLUE® test.

The invention is further described by the following embodiments:

### Methods

In one embodiment, the present invention pertains to a method of producing a population of frozen cells, comprising:
freezing a composition comprising a population of cells, an aqueous component, urea, a monosaccharide, and optionally a bulking agent to produce the population of frozen cells.

In a further embodiment, the present invention pertains to a method of producing a population of reconstituted viable cells, comprising:
(a) freezing a composition comprising a population of cells, an aqueous component, urea, a monosaccharide, and optionally a bulking agent to produce a population of frozen cells; and
(b) resuspending the population of frozen cells in a reconstitution agent to form a reconstituted composition, wherein at least about 1% of the cells are viable.

In a further embodiment, the present invention pertains to a method of producing a population of lyophilized cells, comprising:
(a) freezing a composition comprising a population of cells, an aqueous component, urea, a monosaccharide, and a bulking agent; and
(b) removing at least about 90% of the aqueous component from the frozen composition to produce the population of lyophilized cells.

In a further embodiment, the present invention pertains to a method of producing a population of reconstituted viable cells, comprising:
(a) freezing a composition comprising a population of cells, an aqueous component, urea, a monosaccharide, and a bulking agent;
(b) removing at least about 90% of the aqueous component from the frozen composition to produce the population of lyophilized cells, and
(c) resuspending the population of lyophilized cells in a reconstitution agent to form a reconstituted composition, wherein at least about 1% of the cells are viable.

In any one of the above-embodiments, the composition may further comprise a hydrogel.

In one embodiment, the hydrogel is a biocompatible hydrogel.

In one embodiment, the hydrogel is a hyaluronan gel, alginate gel, agarose gel, collagen gel or combination of thereof.

In any one of the above-embodiments, the population of cells may be suspended in the hydrogel.

In any one of the above-embodiments, the concentration of the monosaccharide prior to freezing may be from about 0.2 M to about 1.0 M.

In any one of the above-embodiments, the concentration of the monosaccharide prior to freezing may be from about 0.3 M to about 0.8 M.

In any one of the above-embodiments, the concentration of the monosaccharide prior to freezing may be from about 0.4 M to about 0.6 M.

In any one of the above-embodiments, the monosaccharide may be glucose, fructose or galactose.

In one embodiment, the monosaccharide is glucose.

In one embodiment, the glucose prior to freezing is from about 0.2 M to about 1.0 M.

In one embodiment, the concentration of the glucose prior to freezing is from about 0.3 M to about 0.8 M.

In one embodiment, the concentration of the glucose prior to freezing is from about 0.4 M to about 0.6 M.

In any one of the above-embodiments, the concentration of the urea prior to freezing may be from about 0.2 M to about 1.0 M.

In any one of the above-embodiments, the concentration of the urea prior to freezing may be from about 0.3 M to about 0.8 M.

In any one of the above-embodiments, the concentration of the urea prior to freezing may be from about 0.4 M to about 0.6 M.

In any one of the above-embodiments, the molar ratio of urea to monosaccharide prior to freezing may be from about 1:3 to about 3:1.

In any one of the above-embodiments, the molar ratio of urea to monosaccharide prior to freezing may be from about 1:2 to about 2:1.

In any one of the above-embodiments, the molar ratio of urea to monosaccharide prior to freezing may be about 1:1.

In any one of the above-embodiments, the molar ratio of urea to glucose prior to freezing may be from about 1:3 to about 3:1.

In any one of the above-embodiments, the molar ratio of urea to glucose prior to freezing may be from about 1:2 to about 2:1.

In any one of the above-embodiments, the molar ratio of urea to glucose prior to freezing may be about 1:1.

In any one of the above-embodiments, the freezing may be performed with the cells in suspension or attached to a surface of a container.

In any one of the above-embodiments, the freezing may be performed with the population of cells suspended in a hydrogel.

In any one of the above-embodiments, the freezing may be performed in a container with or without collagen coating.

In any one of the above-embodiments, the container may be a glass or plastic container with or without a membrane for cell attachment or growth.

In any one of the above-embodiments, the population of cells may be from about 1×10⁴ cells per mL to 1×10⁶ cells per mL.

In any one of the above-embodiments, the population of cells may be from about 1×10⁵ cells per mL to 4×10⁵ cells per mL.

In any one of the above-embodiments, the population of cells may be from about 2×10⁵ cells per mL to 2.5×10⁵ cells per mL.

In any one of the above-embodiments, the bulking agent may comprise a disaccharide.

In one embodiment, the concentration of disaccharide prior to freezing is from about 0.1 M to about 1.0 M.

In one embodiment, the concentration of disaccharide prior to freezing is from about 0.1 M to about 0.5 M.

In one embodiment, the concentration of disaccharide prior to freezing is from about 0.2 M to about 0.4 M.

In one embodiment, the disaccharide is sucrose, lactose, maltose, trehalose, cellobiose, chitobiose, lactulose, isomaltose melibiose or gentiobiose.

In one embodiment, the disaccharide is sucrose.

In one embodiment, the concentration of sucrose prior to freezing is from about 0.1 M to about 1.0 M.

In one embodiment, the concentration of sucrose prior to freezing is from about 0.1 M to about 0.5 M.

In one embodiment, the concentration of sucrose prior to freezing is from about 0.2 M to about 0.4 M.

In any one of the above-embodiments, the bulking agent may comprise a sugar alcohol.

In one embodiment, the concentration of sugar alcohol prior to freezing is from about 0.1 M to about 1.0 M.

In one embodiment, the concentration of sugar alcohol prior to freezing is from about 0.3 M to about 0.8 M.

In one embodiment, the concentration of sugar alcohol prior to freezing is from about 0.4 M to about 0.6 M.

In one embodiment, the sugar alcohol is mannitol, sorbitol, galactitol, fucitol, iditol or inositol.

In one embodiment, the sugar alcohol is mannitol.

In one embodiment, the concentration of mannitol prior to freezing is from about 0.1 M to about 1.0 M.

In one embodiment, the concentration of mannitol prior to freezing is from about 0.3 M to about 0.8 M.

In one embodiment, the concentration of mannitol prior to freezing is from about 0.4 M to about 0.6 M.

In any one of the above-embodiments, the bulking agent may comprise both a disaccharide and a sugar alcohol.

In any one of the above-embodiments, the bulking agent may comprise both sucrose and mannitol.

In any one of the above-embodiments, the composition may not comprise DMSO.

In any one of the above-embodiments, the composition may comprise DMSO at a concentration of from about 1% to about 10% prior to freezing.

In any one of the above-embodiments, the composition may comprise DMSO at a concentration of from about 2% to about 5% prior to freezing.

In any one of the above-embodiments, prior to the freezing in (a), the population of cells may be isolated and contacted with a poration solution.

In one embodiment, the poration solution comprises trehalose and optionally cell culture medium.

In one embodiment, the concentration of trehalose in the poration solution is from about 0.1 M to about 1.0 M.

In one embodiment, the concentration of trehalose in the poration solution is from about 0.2 M to about 0.6 M.

In any one of the above-embodiments, the reconstitution agent may comprise cell culture medium.

In any one of the above-embodiments, the reconstitution agent may comprise a phosphate buffer solution.

In any one of the above-embodiments, the composition may be an isotonic solution.

In any one of the above-embodiments, the composition may be a hypertonic solution.

In any one of the above-embodiments, the population of cells may comprise mammalian cells.

In any one of the above-embodiments, the population of cells may comprise stem cells.

In any one of the above-embodiments, the population of cells may comprise pluripotent stem cells, embryonic stem cells, mesenchymal stem cells, or hematopoietic stem cells.

In any one of the above-embodiments, the population of cells may comprise mesenchymal stem cells.

In any one of the above-embodiments, the population of cells may comprise induced pluripotent stem cells.

In any one of the above-embodiments, the population of cells may comprise neuroblastoma cells.

In one embodiment, the neuroblastoma cells are SK-N-AS cells.

In any one of the above-embodiments, the aqueous component may comprises a buffer.

In one embodiment, the buffer comprises phosphate buffer, Tris buffer, acetate buffer, bicarbonate buffer, histidine buffer, citrate buffer, or combinations thereof.

In any one of the above-embodiments, the aqueous component may comprise cell culture medium.

In one embodiment, the cell culture medium is free of serum.

In any one of the above-embodiments, the removing the aqueous component may comprise lowering pressure, applying heat, or both, to the frozen composition to remove the aqueous component.

In any one of the above-embodiments, the removing the aqueous component may comprise a primary drying step and a secondary drying step.

In any one of the above-embodiments, the removing the aqueous component may comprise only a primary drying step.

In any one of the above-embodiments, the primary drying step may comprise lowering pressure to remove the aqueous component.

In any one of the above-embodiments, the secondary drying step may comprise applying heat to remove the aqueous component.

In any one of the above-embodiments, the freezing may occur at between about −10° C. to about −100° C.

In any one of the above-embodiments, the freezing may occur at between about −20° C. to about −90° C.

In any one of the above-embodiments, the freezing may occur at between about −40° C. to about −60° C.

In any one of the above-embodiments, the freezing lowers the temperature of the composition to about −80° C.

In any one of the above-embodiments, the freezing may lower the temperature of the composition to about −40° C., the aqueous component may be removed at a pressure of a chamber pressure of about 60 mTorr to about 80 mTorr.

In any one of the above-embodiments, the resuspending the frozen cells may occur greater than two hours after freezing.

In any one of the above-embodiments, the resuspending the frozen cells may occur greater than one day after freezing.

In any one of the above-embodiments, the resuspending the frozen cells may occur greater than one week after freezing.

In any one of the above-embodiments, the resuspending the frozen cells may occur greater than one month after freezing.

In any one of the above-embodiments, the resuspending the lyophilized cells may occur greater than two hours after the removing the aqueous component.

In any one of the above-embodiments, the resuspending the lyophilized cells may occur greater than one day after the removing the aqueous component.

In any one of the above-embodiments, the resuspending the lyophilized cells may occur greater than one week after the removing the aqueous component.

In any one of the above-embodiments, the resuspending the lyophilized cells may occur greater than one month after the removing the aqueous component.

In any one of the above-embodiments, the frozen or lyophilized cells may be stored below about −20° C. prior to the resuspending.

In any one of the above-embodiments, the frozen or lyophilized cells may be stored at about −20° C. to about 30° C. prior to the resuspending.

In any one of the above-embodiments, the frozen or lyophilized cells may be stored at about 4° C. to about 28° C. prior to the resuspending.

In any one of the above-embodiments, the frozen or lyophilized cells may be stored at about 10° C. to about 27° C. prior to the resuspending.

In any one of the above-embodiments, the frozen or lyophilized cells may be stored at about 2° C. to about 8° C. prior to the resuspending.

In any one of the above-embodiments, the frozen or lyophilized cells may be stored at greater than about −20° C. for greater than 2 days prior to the resuspending.

In any one of the above-embodiments, the lyophilized cells may be stored at about 20° C. to about 25° C. for greater than 1 week prior to the resuspending.

In any one of the above-embodiments, the lyophilized cells may be stored at about 2° C. to about 8° C. for greater than 1 week prior to the resuspending.

In any one of the above-embodiments, at least about 20% of the cells in the reconstituted composition may be viable as measured by ALAMARBLUE® test.

In any one of the above-embodiments, at least about 30% of the cells in the reconstituted composition may be viable as measured by ALAMARBLUE® test.

In any one of the above-embodiments, at least about 40% of the cells in the reconstituted composition may be viable as measured by ALAMARBLUE® test.

In any one of the above-embodiments, at least about 50% of the cells in the reconstituted composition may be viable as measured by ALAMARBLUE® test.

### Compositions

The present invention further pertains to a composition comprising a population of viable cells, an aqueous component, urea, a monosaccharide, and optionally a bulking agent, wherein the composition contains less than about 10% (vol/vol) water.

In one embodiment, the composition contains less than about 5% (vol/vol) water.

In one embodiment, the molar ratio of glucose to urea in the composition is from about 30:1 to about 1:3.

In one embodiment, the molar ratio of glucose to urea in the composition is from about 15:1 to about 1:2.

In one embodiment, the molar ratio of glucose to urea in the composition is from about 3:1 to about 1:1.

The present invention further pertains to a composition comprising a population of viable cells, an aqueous component, urea, a monosaccharide, and optionally a bulking agent, wherein the molar ratio of glucose to urea in the composition is from about 30:1 to about 1:3.

In one embodiment, the molar ratio of glucose to urea in the composition is from about 15:1 to about 1:2.

In one embodiment, the molar ratio of glucose to urea in the composition is from about 3:1 to about 1:1.

In any one embodiment of the compositions, the monosaccharide may be glucose.

The present invention further pertains to a composition comprising a population of viable cells, an aqueous component, from about 0.2 M to about 1.0 M urea, from about 0.2 M to about 1.0 M glucose, and optionally a bulking agent.

In one embodiment, the composition comprises from about 0.3 M to about 0.8 M glucose.

In one embodiment, the composition comprises from about 0.4 M to about 0.6 M glucose.

In one embodiment, the composition comprises from about 0.3 M to about 0.8 M urea.

In one embodiment, the composition comprises from about 0.4 M to about 0.6 M urea.

In any one embodiment of the compositions, the composition may further comprise sucrose.

In one embodiment, the composition comprises from about 0.1 M to about 0.5 M sucrose.

In any one embodiment of the compositions, the composition may further comprise mannitol.

In one embodiment, the composition comprises from about 0.3 M to about 0.8 M mannitol.

In any one embodiment of the compositions, the population of viable cells may comprise mammalian cells.

In any one embodiment of the compositions, the population of viable cells may comprise stem cells.

In any one embodiment of the compositions, the population of viable cells may comprise pluripotent stem cells, embryonic stem cells, mesenchymal stem cells, or hematopoietic stem cells.

In any one embodiment of the compositions, the population of viable cells may comprise mesenchymal stem cells.

In any one embodiment of the compositions, the population of viable cells may comprise induced pluripotent stem cells.

In any one embodiment of the compositions, the population of viable cells may comprise neuroblastoma cells.

In one embodiment, the neuroblastoma cells are SK-N-AS cells.

In any one embodiment of the compositions, the composition may be free of DMSO.

### Methods

The present invention further pertains to a method of freezing a population of cells, comprising freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.

The present invention further pertains to a method of lyophilizing a population of cells, comprising:
(a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and
(b) removing the aqueous component from the frozen composition to produce a population of lyophilized cells, wherein the aqueous component in the population of lyophilized cells is less than about 10% w/w.

The present invention further pertains to a method of producing a viable population of cells, comprising:
(a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and
(b) resuspending the population of lyophilized cells in a reconstitution agent to form a reconstituted composition, wherein at least about 1% of the cells in the reconstituted composition are viable.

The present invention further pertains to a method of producing a viable population of cells, comprising:
(a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.;
(b) removing the aqueous component from the frozen composition to produce a population of lyophilized cells, wherein the aqueous component in the population of lyophilized cells is less than about 10% w/w; and
(c) resuspending the population of lyophilized cells in a reconstitution agent to form a reconstituted composition, wherein at least about 1% of the cells in the reconstituted composition are viable.

The present invention further pertains to a method of freezing a population of cells, comprising freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.

The present invention further pertains to a method of lyophilizing a population of cells, comprising:
(a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; and
(b) removing the aqueous component from the frozen composition to produce a population of lyophilized cells, wherein the aqueous component in the population of lyophilized cells is less than about 10% w/w.

The present invention further pertains to a method of producing a viable population of cells, comprising:
(a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; and
(b) resuspending the population of lyophilized cells in a reconstitution agent to form a reconstituted composition, wherein at least about 1% of the cells in the reconstituted composition are viable.

The present invention further pertains to a method of producing a viable population of cells, comprising:
(a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.;
(b) removing the aqueous component from the frozen composition to produce a population of lyophilized cells, wherein the aqueous component in the population of lyophilized cells is less than about 10% w/w; and
(c) resuspending the population of lyophilized cells in a reconstitution agent to form a reconstituted composition, wherein at least about 1% of the cells in the reconstituted composition are viable.

In any one embodiment of the methods, the composition may further comprise from about 1% to about 8% DMSO.

In any one embodiment of the methods, the composition may further comprise a hydrogel.

In one embodiment, the hydrogel is a hyaluronan gel, alginate gel or collagen gel.

In one embodiment, the population of cells is suspended in the hydrogel.

In any one embodiment of the methods, the composition may be free of DMSO.

In any one embodiment of the methods, at least about 20% of the cells in the reconstituted composition may be viable as measured by ALAMARBLUE® test.

In any one embodiment of the methods, at least about 30% of the cells in the reconstituted composition may be viable as measured by ALAMARBLUE® test.

In any one embodiment of the methods, at least about 40% of the cells in the reconstituted composition may be viable as measured by ALAMARBLUE® test.

In any one embodiment of the methods, at least about 50% of the cells in the reconstituted composition may be viable as measured by ALAMARBLUE® test.

### Items

What is itemed is:
1. A method of producing a population of frozen cells, comprising: freezing a composition comprising a population of cells, an aqueous component, urea, a monosaccharide, and optionally a bulking agent to produce the population of frozen cells.
2. A method of producing a population of reconstituted viable cells, comprising:
   (a) freezing a composition comprising a population of cells, an aqueous component, urea, a monosaccharide, and optionally a bulking agent to produce a population of frozen cells; and
   (b) resuspending the population of frozen cells in a reconstitution agent to form a reconstituted composition, wherein at least about 1% of the cells are viable.
3. A method of producing a population of lyophilized cells, comprising:
   (a) freezing a composition comprising a population of cells, an aqueous component, urea, a monosaccharide, and a bulking agent; and
   (b) removing at least about 90% of the aqueous component from the frozen composition to produce the population of lyophilized cells.
4. A method of producing a population of reconstituted viable cells, comprising:
   (a) freezing a composition comprising a population of cells, an aqueous component, urea, a monosaccharide, and a bulking agent;
   (b) removing at least about 90% of the aqueous component from the frozen composition to produce the population of lyophilized cells, and
   (c) resuspending the population of lyophilized cells in a reconstitution agent to form a reconstituted composition, wherein at least about 1% of the cells are viable.
5. The method of any one of items 1 to 4, wherein the composition further comprises a hydrogel.
6. The method of item 5, wherein the hydrogel is a biocompatible hydrogel.
7. The method of item 5, wherein the hydrogel is a hyaluronan gel, alginate gel, agarose gel, collagen gel or combination of thereof.
8. The method of any one of items 1 to 7, wherein the population of cells is suspended in the hydrogel.
9. The method of any one of items 1 to 8, wherein the concentration of the monosaccharide prior to freezing is from about 0.2 M to about 1.0 M.
10. The method of any one of items 1 to 8, wherein the concentration of the monosaccharide prior to freezing is from about 0.3 M to about 0.8 M.
11. The method of any one of items 1 to 8, wherein the concentration of the monosaccharide prior to freezing is from about 0.4 M to about 0.6 M.
12. The method of any one of items 1 to 11, where in the monosaccharide is glucose, fructose or galactose.
13. The method of item 12, wherein the monosaccharide is glucose.
14. The method of item 13, wherein the concentration of the glucose prior to freezing is from about 0.2 M to about 1.0 M.
15. The method of item 13, wherein the concentration of the glucose prior to freezing is from about 0.3 M to about 0.8 M.
16. The method of item 13, wherein the concentration of the glucose prior to freezing is from about 0.4 M to about 0.6 M.
17. The method of any one of items 1 to 16, wherein the concentration of the urea prior to freezing is from about 0.2 M to about 1.0 M.
18. The method of any one of items 1 to 16, wherein the concentration of the urea prior to freezing is from about 0.3 M to about 0.8 M.
19. The method of any one of items 1 to 16, wherein the concentration of the urea prior to freezing is from about 0.4 M to about 0.6 M.
20. The method of any one of items 1 to 19, wherein the ratio of urea to monosaccharide prior to freezing is from about 1:3 to about 3:1.
21. The method of any one of items 1 to 19, wherein the ratio of urea to monosaccharide prior to freezing is from about 1:2 to about 2:1.
22. The method of any one of items 1 to 19, wherein the ratio of urea to monosaccharide prior to freezing is about 1:1.
23. The method of any one of items 13 to 19, wherein the ratio of urea to glucose prior to freezing is from about 1:3 to about 3:1.
24. The method of any one of items 1 to 19, wherein the ratio of urea to glucose prior to freezing is from about 1:2 to about 2:1.
25. The method of any one of items 1 to 19, wherein the ratio of urea to glucose prior to freezing is about 1:1.
26. The method of any one of items 1 to 25, wherein the freezing is performed with the cells in suspension or attached to a surface of a container.
27. The method of any one of items 1 to 25, wherein the freezing is performed with the population of cells suspended in a hydrogel.
28. The method of any one of items 1 to 27, wherein the freezing is performed in a container with or without collagen coating.
29. The method of any one of items 1 to 28, wherein the container is a glass or plastic container with or without a membrane for cell attachment or growth.
30. The method of any one of items 1 to 29, wherein the population of cells is from about 1×10⁴ cells per mL to 1×10⁷ cells per mL.
31. The method of any one of items 1 to 29, wherein the population of cells is from about 1×10⁵ cells per mL to 4×10⁵ cells per mL.
32. The method of any one of items 1 to 29, wherein the population of cells is from about 2×10⁵ cells per mL to 2.5×10⁵ cells per mL.
33. The method of any one of items 1 to 32, wherein the bulking agent comprises a disaccharide.
34. The method item 33, wherein the concentration of disaccharide prior to freezing is from about 0.1 M to about 1.0 M.
35. The method item 33, wherein the concentration of disaccharide prior to freezing is from about 0.1 M to about 0.5 M.
36. The method item 33, wherein the concentration of disaccharide prior to freezing is from about 0.2 M to about 0.4 M.
37. The method of any one of items 33 to 36, wherein the disaccharide is sucrose, lactose, maltose, trehalose, cellobiose, chitobiose, lactulose, isomaltose melibiose or gentiobiose.
38. The method of item 33, wherein the disaccharide is sucrose.
39. The method item 35, wherein the concentration of sucrose prior to freezing is from about 0.1 M to about 1.0 M.
40. The method item 35, wherein the concentration of sucrose prior to freezing is from about 0.1 M to about 0.5 M.
41. The method item 35, wherein the concentration of sucrose prior to freezing is from about 0.2 M to about 0.4 M.
42. The method of any one of items 1 to 32, wherein the bulking agent comprises a sugar alcohol.
43. The method item 42, wherein the concentration of sugar alcohol prior to freezing is from about 0.1 M to about 1.0 M.
44. The method item 42, wherein the concentration of sugar alcohol prior to freezing is from about 0.3 M to about 0.8 M.
45. The method item 42, wherein the concentration of sugar alcohol prior to freezing is from about 0.4 M to about 0.6 M.
46. The method of any one of items 42 to 45, wherein the sugar alcohol is mannitol, sorbitol, galactitol, fucitol, iditol or inositol.
47. The method of item 46, wherein the sugar alcohol is mannitol.
48. The method item 47, wherein the concentration of mannitol prior to freezing is from about 0.1 M to about 1.0 M.
49. The method item 47, wherein the concentration of mannitol prior to freezing is from about 0.3 M to about 0.8 M.
50. The method item 47, wherein the concentration of mannitol prior to freezing is from about 0.4 M to about 0.6 M.
51. The method of any one of items 1 to 50, wherein the bulking agent comprises both a disaccharide and a sugar alcohol.
52. The method of any one of items 1 to 50, wherein the bulking agent comprises both sucrose and mannitol.
53. The method of any one of items 1 to 52, wherein the composition does not comprise DMSO.
54. The method of any one of items 1 to 52, wherein the composition comprises DMSO at a concentration of from about 1% to about 10% prior to freezing.
55. The method of any one of items 1 to 52, wherein the composition comprises DMSO at a concentration of from about 2% to about 5% prior to freezing.
56. The method of any one of items 1 to 55, wherein prior to the freezing in (a), the population of cells is isolated and contacted with a poration solution.
57. The method of item 56, wherein the poration solution comprises trehalose and cell culture medium.
58. The method of item 57, wherein the concentration of trehalose in the poration solution is from about 0.1 M to about 1.0 M.
59. The method of item 57, wherein the concentration of trehalose in the poration solution is from about 0.2 M to about 0.6 M.
60. The method of any one of items 2 or 4 to 59, wherein the reconstitution agent comprises cell culture medium.
61. The method of any one of items 2 or 4 to 62, wherein the reconstitution agent comprises a phosphate buffer solution.
62. The method of any one of items 1 to 62, wherein the composition is an isotonic solution.
63. The method of any one of items 1 to 63, wherein the composition is a hypertonic solution.
64. The method of any one of items 1 to 64, wherein the population of cells comprises mammalian cells.
65. The method of any one of items 1 to 65, wherein the population of cells comprises stem cells.
66. The method of any one of items 1 to 66, wherein the population of cells comprises pluripotent stem cells, embryonic stem cells, mesenchymal stem cells, or hematopoietic stem cells.
67. The method of any one of items 1 to 67, wherein the population of cells comprises mesenchymal stem cells.
68. The method of any one of items 1 to 68, wherein the population of cells comprises induced pluripotent stem cells.
69. The method of any one of items 1 to 67, wherein the population of cells comprises neuroblastoma cells.
70. The method of item 70, wherein the neuroblastoma cells are SK-N-AS cells.
71. The method of any one of items 1 to 71, wherein the aqueous component comprises a buffer.
72. The method of item 72, wherein the buffer comprises phosphate buffer, Tris buffer, acetate buffer, bicarbonate buffer, histidine buffer, citrate buffer, or combinations thereof.
73. The method of any one of items 1 to 73, wherein the aqueous component comprises cell culture medium.
74. The method of item 74, wherein the cell culture medium is free of serum.
75. The method of any one of items 3 to 75, wherein the removing the aqueous component comprises lowering pressure, applying heat, or both, to the frozen composition to remove the aqueous component.
76. The method of any one of items 3 to 76, wherein the removing the aqueous component comprises a primary drying step and a secondary drying step.
77. The method of any one of items 3 to 77, wherein the removing the aqueous component comprises only a primary drying step.
78. The method of any one of items 3 to 78, wherein the primary drying step comprises lowering pressure to remove the aqueous component.
79. The method of any one of items 3 to 79, wherein the secondary drying step comprises applying heat to remove the aqueous component.
80. The method of any one of items 1 to 80, wherein the freezing occurs at between about −10° C. to about −100° C.
81. The method of any one of items 1 to 80, wherein the freezing occurs at between about −20° C. to about −90° C.
82. The method of any one of items 1 to 80, wherein the freezing occurs at between about −40° C. to about −60° C.
83. The method of any one of items 1 to 80, wherein the freezing lowers the temperature of the composition to about −80° C.
84. The method of any one of items 3 to 84, wherein the freezing lowers the temperature of the composition to about −40° C., the aqueous component is removed at a pressure of a chamber pressure of about 60 mTorr to about 80 mTorr.
85. The method of any one of items 2 or 5 to 85, wherein the resuspending the frozen cells occurs greater than two hours after freezing.
86. The method of any one of items 2 or 5 to 85, wherein the resuspending the frozen cells occurs greater than one day after freezing.
87. The method of any one of items 2 or 5 to 85, wherein the resuspending the frozen cells occurs greater than one week after freezing.
88. The method of any one of items 2 or 5 to 85, wherein the resuspending the frozen cells occurs greater than one month after freezing.
89. The method of any one of items 4 to 85, wherein the resuspending the lyophilized cells occurs greater than two hours after the removing the aqueous component.
90. The method of any one of items 4 to 85, wherein the resuspending the lyophilized cells occurs greater than one day after the removing the aqueous component.
91. The method of any one of items 4 to 85, wherein the resuspending the lyophilized cells occurs greater than one week after the removing the aqueous component.
92. The method of any one of items 4 to 85, wherein the resuspending the lyophilized cells occurs greater than one month after the removing the aqueous component.
93. The method of any one of items 2 or 4 to 93, wherein the frozen or lyophilized cells are stored below about −20° C. prior to the resuspending.
94. The method of any one of items 2 or 4 to 93, wherein the frozen or lyophilized cells are stored at about −20° C. to about 30° C. prior to the resuspending.
95. The method of any one of items 2 or 4 to 93, wherein the frozen or lyophilized cells are stored at about 4° C. to about 28° C. prior to the resuspending.
96. The method of any one of items 2 or 4 to 93, wherein the frozen or lyophilized cells are stored at about 10° C. to about 27° C. prior to the resuspending.
97. The method of any one of items 2 or 4 to 93, wherein the frozen or lyophilized cells are stored at about 2° C. to about 8° C. prior to the resuspending.
98. The method of any one of items 2 or 4 to 93 wherein the frozen or lyophilized cells are stored at greater than about −20° C. for greater than 2 days prior to the resuspending.
99. The method of any one of items 4 to 98, wherein the lyophilized cells are stored at about 20° C. to about 25° C. for greater than 1 week prior to the resuspending.
100. The method of any one of items 4 to 98, wherein the lyophilized cells are stored at about 2° C. to about 8° C. for greater than 1 week prior to the resuspending.
101. The method of any one of items 1 to 101, wherein at least about 20% of the cells in the reconstituted composition are viable.
102. The method of any one of items 1 to 101, wherein at least about 30% of the cells in the reconstituted composition are viable.
103. The method of any one of items 1 to 101, wherein at least about 40% of the cells in the reconstituted composition are viable.
104. The method of any one of items 1 to 101, wherein at least about 50% of the cells in the reconstituted composition are viable as measured by ALAMARBLUE test.
105. A composition comprising a population of viable cells, an aqueous component, urea, a monosaccharide, and a bulking agent, wherein the composition contains less than about 10% (vol/vol) water.
106. The composition of item 106, wherein the composition contains less than about 5% (vol/vol) water.
107. The composition of item 107, wherein the weight ratio of glucose to urea in the composition is from about 30:1 to about 1:3.
108. The composition of item 107, wherein the weight ratio of glucose to urea in the composition is from about 15:1 to about 1:2.
109. The composition of item 107, wherein the weight ratio of glucose to urea in the composition is from about 3:1 to about 1:1.
110. A composition comprising a population of viable cells, an aqueous component, urea, a monosaccharide, and optionally a bulking agent, wherein the weight ratio of glucose to urea in the composition is from about 30:1 to about 1:3.
111. The composition of item 111, wherein the weight ratio of glucose to urea in the composition is from about 15:1 to about 1:2.
112. The composition of item 111, wherein the weight ratio of glucose to urea in the composition is from about 3:1 to about 1:1.
113. The composition of any of items 107 to 113, wherein the monosaccharide is glucose.
114. A composition comprising a population of viable cells, an aqueous component, from about 0.2 M to about 1.0 M urea, from about 0.2 M to about 1.0 M glucose, and optionally a bulking agent.
115. The composition of item 115, comprising from about 0.3 M to about 0.8 M glucose.
116. The composition of item 115, comprising from about 0.4 M to about 0.6 M glucose.
117. The composition of any one of items 115 to 117, comprising from about 0.3 M to about 0.8 M urea.
118. The composition of any one of items 115 to 117, comprising from about 0.4 M to about 0.6 M urea.
119. The composition of any one of items 115 to 117, further comprising sucrose.
120. The composition of item 120, comprising from about 0.1 M to about 0.5 M sucrose.
121. The composition of any one of items 115 to 117, further comprising mannitol.
122. The composition of item 122, comprising from about 0.3 M to about 0.8 M mannitol.
123. The composition of any one of items 107 to 123, wherein the population of viable cells comprises mammalian cells.
124. The composition of any one of items 107 to 123, wherein the population of viable cells comprises stem cells.
125. The composition of any one of items 107 to 123, wherein the population of viable cells comprises pluripotent stem cells, embryonic stem cells, mesenchymal stem cells, or hematopoietic stem cells.
126. The composition of any one of items 107 to 123, wherein the population of viable cells comprises mesenchymal stem cells.
127. The composition of any one of items 107 to 123, wherein the population of viable cells comprises induced pluripotent stem cells.
128. The composition of any one of items 107 to 123, wherein the population of viable cells comprises neuroblastoma cells.
129. The composition of item 129, wherein the neuroblastoma cells are SK-N-AS cells.
130. The composition of any one of items 107 to 130, wherein the composition is free of DMSO.
131. A method of freezing a population of cells, comprising freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.
132. A method of lyophilizing a population of cells, comprising:
   (a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and
   (b) removing the aqueous component from the frozen composition to produce a population of lyophilized cells, wherein the aqueous component in the population of lyophilized cells is less than about 10% w/w.
133. A method of producing a viable population of cells, comprising:
   (a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.; and
   (b) resuspending the population of lyophilized cells in a reconstitution agent to form a reconstituted composition, wherein at least about 1% of the cells in the reconstituted composition are viable.
134. A method of producing a viable population of cells, comprising:
   (a) freezing a composition comprising the population of cells, an aqueous component, about 0.2 M to about 1 M urea, about 0.2 M to about 1 M glucose, and about 0.1 M to about 0.5 M sucrose, wherein the freezing occurs below about −30° C.;
   (b) removing the aqueous component from the frozen composition to produce a population of lyophilized cells, wherein the aqueous component in the population of lyophilized cells is less than about 10% w/w; and
   (c) resuspending the population of lyophilized cells in a reconstitution agent to form a reconstituted composition, wherein at least about 1% of the cells in the reconstituted composition are viable.
135. A method of freezing a population of cells, comprising freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.
136. A method of lyophilizing a population of cells, comprising:
   (a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; and
   (b) removing the aqueous component from the frozen composition to produce a population of lyophilized cells, wherein the aqueous component in the population of lyophilized cells is less than about 10% w/w.
137. A method of producing a viable population of cells, comprising:
   (a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.; and
   (b) resuspending the population of lyophilized cells in a reconstitution agent to form a reconstituted composition, wherein at least about 1% of the cells in the reconstituted composition are viable.
138. A method of producing a viable population of cells, comprising:
   (a) freezing a composition comprising the population of cells, an aqueous component, about 0.2 M to about 1 M urea, about 0.2 M to about 1 M glucose, and about 0.2 M to about 0.8 M mannitol, wherein the freezing occurs below about −30° C.;
   (b) removing the aqueous component from the frozen composition to produce a population of lyophilized cells, wherein the aqueous component in the population of lyophilized cells is less than about 10% w/w; and
   (c) resuspending the population of lyophilized cells in a reconstitution agent to form a reconstituted composition, wherein at least about 1% of the cells in the reconstituted composition are viable.
139. The method of any one of items 132 to 138, wherein the composition further comprises from about 1% to about 8% DMSO.
140. The method of any one of items 132 to 139, wherein the composition further comprises a hydrogel.
141. The method of item 140, wherein the hydrogel is a hyaluronan gel, alginate gel or collagen gel.
142. The method of item 141, wherein the population of cells is suspended in the hydrogel.
143. The method of any one of items 132 to 142, wherein the composition is free of DMSO.
144. The method of any one of items 133, 134, 137 or 138 to 143 to, wherein at least about 20% of the cells in the reconstituted composition are viable.
145. The method of any one of items 133, 134, 137 or 138 to 143, wherein at least about 30% of the cells in the reconstituted composition are viable.
146. The method of any one of items 133, 134, 137 or 138 to 143, wherein at least about 40% of the cells in the reconstituted composition are viable.
147. The method of any one of items 133, 134, 137 or 138 to 143, wherein at least about 50% of the cells in the reconstituted composition are viable as measured by ALAMARBLUE test.
   The present invention further pertains to the following numbered embodiments:
1. A method of producing a population of frozen cells, comprising: freezing a composition comprising a population of cells, an aqueous component, urea, a monosaccharide, and optionally a bulking agent to produce the population of frozen cells.
2. A method of producing a population of reconstituted viable cells, comprising:
   (a) freezing a composition comprising a population of cells, an aqueous component, urea, a monosaccharide, and optionally a bulking agent to produce a population of frozen cells; and
   (b) resuspending the population of frozen cells in a reconstitution agent to form a reconstituted composition, wherein at least about 1% of the cells are viable.
3. A method of producing a population of lyophilized cells, comprising:
   (a) freezing a composition comprising a population of cells, an aqueous component, urea, a monosaccharide, and a bulking agent; and
   (b) removing at least about 90% of the aqueous component from the frozen composition to produce the population of lyophilized cells.
4. A method of producing a population of reconstituted viable cells, comprising:
   (a) freezing a composition comprising a population of cells, an aqueous component, urea, a monosaccharide, and a bulking agent;
   (b) removing at least about 90% of the aqueous component from the frozen composition to produce the population of lyophilized cells, and
   (c) resuspending the population of lyophilized cells in a reconstitution agent to form a reconstituted composition, wherein at least about 1% of the cells are viable.
5. The method of any one of embodiments 1 to 4, wherein the composition further comprises a hydrogel, preferably, wherein the hydrogel is a biocompatible hydrogel, or a hyaluronan gel, alginate gel, agarose gel, collagen gel or combination of thereof.
6. The method of any one of embodiments 1 to 5, wherein the population of cells is suspended in the hydrogel.
7. The method of any one of embodiments 1 to 6, wherein the concentration of the monosaccharide prior to freezing is from about 0.2 M to about 1.0 M, or from about 0.3 M to about 0.8 M, or from about 0.4 M to about 0.6 M.
8. The method of any one of embodiments 1 to 7, where in the monosaccharide is glucose, fructose or galactose, preferably, wherein the monosaccharide is glucose, optionally, wherein the concentration of the glucose prior to freezing is from about 0.2 M to about 1.0 M, or from about 0.3 M to about 0.8 M, or from about 0.4 M to about 0.6 M.
9. The method of any one of embodiments 1 to 8, wherein the concentration of the urea prior to freezing is from about 0.2 M to about 1.0 M, preferably, from about 0.3 M to about 0.8 M, or from about 0.4 M to about 0.6 M, and/or wherein the ratio of urea to monosaccharide prior to freezing is from about 1:3 to about 3:1, preferably from about 1:2 to about 2:1, or is about 1:1.
10. The method of embodiment 8, wherein the ratio of urea to glucose prior to freezing is from about 1:3 to about 3:1, preferably from about 1:2 to about 2:1, or is about 1:1.
11. The method of any one of embodiments 1 to 10, wherein the freezing is performed with the cells in suspension or attached to a surface of a container, or with the population of cells suspended in a hydrogel, and/or in a container with or without collagen coating, optionally, wherein the container is a glass or plastic container with or without a membrane for cell attachment or growth.
12. The method of any one of the preceding embodiments, wherein the population of cells is from about 1×10⁴ cells per mL to 1×10⁷ cells per mL, or from about 1×10⁵ cells per mL to 4×10⁵ cells per mL, or from about 2×10⁵ cells per mL to 2.5×10⁵ cells per mL.
13. The method of any one of the preceding embodiments, wherein the bulking agent comprises
   a) a disaccharide,
   optionally, wherein the concentration of disaccharide prior to freezing is from about 0.1 M to about 1.0 M, or from about 0.1 M to about 0.5 M, or from about 0.2 M to about 0.4 M,
   optionally, wherein the disaccharide is sucrose, lactose, maltose, trehalose, cellobiose, chitobiose, lactulose, isomaltose melibiose or gentiobiose, preferably, wherein the disaccharide is sucrose, optionally, wherein the concentration of sucrose prior to freezing is from about 0.1 M to about 1.0, or from about 0.1 M to about 0.5 M, or from about 0.2 M to about 0.4 M; or
   b) a sugar alcohol,
   optionally, wherein the concentration of sugar alcohol prior to freezing is from about 0.1 M to about 1.0 M, or from about 0.3 M to about 0.8 M, or from about 0.4 M to about 0.6 M,
   optionally, wherein the sugar alcohol is mannitol, sorbitol, galactitol, fucitol, iditol or inositol, preferably, wherein the sugar alcohol is mannitol, optionally, wherein the concentration of mannitol prior to freezing is from about 0.1 M to about 1.0 M, or from about 0.3 M to about 0.8 M, or about 0.4 M to about 0.6 M; or
   c) both a disaccharide and a sugar alcohol; or
   d) both sucrose and mannitol.
14. The method of any one of the preceding embodiments, wherein the composition does not comprise DMSO, or, wherein the composition comprises DMSO at a concentration of from about 1% to about 10% prior to freezing, or wherein the composition comprises DMSO at a concentration of from about 2% to about 5% prior to freezing.
15. The method of any one of the preceding embodiments, wherein prior to the freezing in (a), the population of cells is isolated and contacted with a poration solution, preferably, wherein the poration solution comprises trehalose and cell culture medium, optionally, wherein the concentration of trehalose in the poration solution is from about 0.1 M to about 1.0 M, or from about 0.2 M to about 0.6 M.
16. The method of any one of embodiments 2 or 4 to 15, wherein the reconstitution agent comprises cell culture medium, or wherein the reconstitution agent comprises a phosphate buffer solution.
17. The method of any one of the preceding embodiments, wherein the composition is an isotonic solution, or wherein the composition is a hypertonic solution.
18. The method of any one of the preceding embodiments, wherein the population of cells comprises mammalian cells, preferably, wherein the population of cells comprises stem cells, optionally wherein the population of cells comprises pluripotent stem cells, embryonic stem cells, mesenchymal stem cells, or hematopoietic stem cell, and/or wherein the population of cells comprises mesenchymal stem cells, and/or wherein the population of cells comprises induced pluripotent stem cells, and/or wherein the population of cells comprises neuroblastoma cells, preferably, wherein the neuroblastoma cells are SK-N-AS cells.
19. The method of any one of the preceding embodiments, wherein the aqueous component comprises
   a) a buffer, preferably, wherein the buffer comprises phosphate buffer, Tris buffer, acetate buffer, bicarbonate buffer, histidine buffer, citrate buffer, or combinations thereof, and/or
   b) cell culture medium, preferably, wherein the cell culture medium is free of serum.
20. The method of any one of embodiments 3 to 19, wherein the removing the aqueous component comprises lowering pressure, applying heat, or both, to the frozen composition to remove the aqueous component, and/or wherein the removing the aqueous component comprises a primary drying step and a secondary drying step, and/or wherein the removing the aqueous component comprises only a primary drying step, preferably, wherein the primary drying step comprises lowering pressure to remove the aqueous component, and/or wherein the secondary drying step comprises applying heat to remove the aqueous component.
21. The method of any one of the preceding embodiments, wherein the freezing occurs at between about −10° C. to about −100° C., preferably, at between about −20° C. to about −90°, or at between about −40° C. to about −60° C., or wherein the freezing lowers the temperature of the composition to about −80° C., preferably wherein the freezing lowers the temperature of the composition to about −40° C., the aqueous component is removed at a pressure of a chamber pressure of about 60 mTorr to about 80 mTorr.
22. The method of any one of embodiments 2 or 5 to 21, wherein the resuspending the frozen cells occurs greater than two hours after freezing, preferably, greater than one day after freezing, or greater than one week after freezing, or greater than one month after freezing.
23. The method of any one of embodiments 4 to 22, wherein the resuspending the lyophilized cells occurs greater than two hours after the removing the aqueous component, preferably, greater than one day after the removing the aqueous component, or greater than one week after the removing the aqueous component, or greater than one month after the removing the aqueous component.
24. The method of any one of embodiments 2 or 4 to 23, wherein the frozen or lyophilized cells are stored below about −20° C. prior to the resuspending, preferably at about −20° C. to about 30° C. prior to the resuspending, or at about 4° C. to about 28° C. prior to the resuspending, or at about 10° C. to about 27° C. prior to the resuspending, or at about 2° C. to about 8° C. prior to the resuspending, or at greater than about −20° C. for greater than 2 days prior to the resuspending.
25. The method of any one of embodiments 4 to 24, wherein the lyophilized cells are stored at about 20° C. to about 25° C. for greater than 1 week prior to the resuspending, preferably at about 2° C. to about 8° C. for greater than 1 week prior to the resuspending.
26. The method of any one of the preceding embodiments, wherein at least about 20% of the cells in the reconstituted composition are viable, preferably, wherein at least about 30% of the cells in the reconstituted composition are viable, or wherein at least about 40% of the cells in the reconstituted composition are viable, or wherein at least about 50% of the cells in the reconstituted composition are viable as measured by ALAMARBLUE test.
27. A composition comprising a population of viable cells, an aqueous component, urea, a monosaccharide, and a bulking agent, wherein the composition contains less than about 10% (vol/vol) water, preferably, less than about 5% (vol/vol) water, optionally, wherein the weight ratio of glucose to urea in the composition is from about 30:1 to about 1:3, or from about 15:1 to about 1:2, or from about 3:1 to about 1:1.
28. A composition comprising a population of viable cells, an aqueous component, urea, a monosaccharide, and optionally a bulking agent, wherein the weight ratio of glucose to urea in the composition is from about 30:1 to about 1:3, optionally, wherein the weight ratio of glucose to urea in the composition is from about 15:1 to about 1:2, or from about 3:1 to about 1:1.
29. The composition of any of embodiments 27 to 28, wherein the monosaccharide is glucose.
30. A composition comprising a population of viable cells, an aqueous component, from about 0.2 M to about 1.0 M urea, from about 0.2 M to about 1.0 M glucose, and optionally a bulking agent, optionally comprising from about 0.3 M to about 0.8 M glucose, or from about 0.4 M to about 0.6 M glucose, and/or from about 0.3 M to about 0.8 M urea, or from about 0.4 M to about 0.6 M urea, and/or further comprising sucrose, preferably from about 0.1 M to about 0.5 M sucrose.
   The present invention also pertains to the following numbered embodiments:
1. A method of producing a population of frozen cells, comprising: freezing a composition comprising a population of cells, an aqueous component, urea, a monosaccharide, and optionally a bulking agent to produce the population of frozen cells.
2. A method of producing a population of reconstituted viable cells, comprising:
   (a) freezing a composition comprising a population of cells, an aqueous component, urea, a monosaccharide, and optionally a bulking agent to produce a population of frozen cells; and
   (b) resuspending the population of frozen cells in a reconstitution agent to form a reconstituted composition, wherein at least about 1% of the cells are viable.
3. The method of embodiment 1 or 2, wherein the concentration of the monosaccharide prior to freezing is from about 0.2 M to about 1.25 M, or from about 0.3 M to about 0.8 M, or from about 0.4 M to about 0.6 M.
4. The method of any one of embodiments 1 to 3, where in the monosaccharide is glucose, fructose or galactose.
5. The method of any one of embodiments 1 to 4, wherein the monosaccharide is glucose and wherein the concentration of the glucose prior to freezing is from about 0.2 M to about 1.25 M, or from about 0.2 M to about 0.8 M, or from about 0.4 M to about 0.6 M.
6. The method of any one of embodiments 1 to 5, wherein the concentration of the urea prior to freezing is from about 0.2 M to about 1.25 M, or from about 0.2 M to about 0.8 M, or from about 0.4 M to about 0.6 M.
7. The method of any one of embodiments 1 to 6, wherein the molar ratio of urea to monosaccharide prior to freezing is from about 1:3 to about 3:1, or from about 1:2 to about 2:1, or is about 1:1.
8. The method of any one of embodiments 1 to 7, wherein the monosaccharide is glucose and the molar ratio of urea to glucose prior to freezing is from about 1:3 to about 3:1, or from about 1:2 to about 2:1, or is about 1:1.
9. A method of producing a population of lyophilized cells, comprising:
   (a) freezing a composition comprising a population of cells, a hyaluronan gel, an aqueous component, urea, a monosaccharide, and a bulking agent, wherein the concentration of the monosaccharide prior to freezing is at least about 0.6 M, and the concentration of urea prior to freezing is at least about 0.6 M; and
   (b) removing at least about 90% of the aqueous component from the frozen composition to produce the population of lyophilized cells.
10. A method of producing a population of reconstituted viable cells, comprising:
   (a) freezing a composition comprising a population of cells, a hyaluronan gel, an aqueous component, urea, a monosaccharide, and a bulking agent, wherein the concentration of the monosaccharide prior to freezing is at least about 0.6 M, and the concentration of urea prior to freezing is at least about 0.6 M;
   (b) removing at least about 90% of the aqueous component from the frozen composition to produce the population of lyophilized cells, and
   (c) resuspending the population of lyophilized cells in a reconstitution agent to form a reconstituted composition, wherein at least about 1% of the cells are viable.
11. The method of embodiments 9 or 10, wherein the concentration of the monosaccharide prior to freezing is from about 0.6 M to about 2.0 M, or from about 0.7 M to about 1.7 M, or from about 0.8 M to about 1.4 M.
12. The method of any one of embodiments 9 to 11, where in the monosaccharide is glucose, fructose or galactose,
13. The method of any one of embodiments 9 to 12, wherein the monosaccharide is glucose and wherein the concentration of the glucose prior to freezing is from about 0.6 M to about 2.0 M, or from about 0.7 M to about 1.7 M, or from about 0.8 M to about 1.4 M.
14. The method of any one of embodiments 9 to 13, wherein the concentration of the urea prior to freezing is from about 0.6 M to about 2.0 M, or from about 0.7 M to about 1.7 M, or from about 0.8 M to about 1.4 M.
15. The method of any one of embodiments 9 to 14, wherein the molar ratio of urea to monosaccharide prior to freezing is from about 1:3 to about 3:1, or from about 1:2 to about 2:1, or is about 1:1.
16. The method of any one of embodiments 9 to 15, wherein the monosaccharide is glucose and the molar ratio of urea to glucose prior to freezing is from about 1:3 to about 3:1, or from about 1:2 to about 2:1, or is about 1:1.
17. The method of any one of the preceding embodiments, wherein the bulking agent comprises
   a) a disaccharide,
   wherein the concentration of disaccharide prior to freezing is from about 0.1 M to about 1.0 M, or from about 0.1 M to about 0.5 M, or from about 0.2 M to about 0.4 M,
   wherein the disaccharide is sucrose, lactose, maltose, trehalose, cellobiose, chitobiose, lactulose, isomaltose melibiose or gentiobiose; or
   b) a sugar alcohol,
   wherein the concentration of sugar alcohol prior to freezing is from about 0.1 M to about 1.0 M, or from about 0.3 M to about 0.8 M, or from about 0.4 M to about 0.6 M,
   wherein the sugar alcohol is mannitol, sorbitol, galactitol, fucitol, iditol or inositol; or
   c) both a disaccharide and a sugar alcohol; or
   d) both sucrose and mannitol.
18. The method of any one of the preceding embodiments, wherein the composition does not comprise DMSO, or, wherein the composition comprises DMSO at a concentration of from about 1% to about 10% prior to freezing, or wherein the composition comprises DMSO at a concentration of from about 1% to about 5% prior to freezing.
19. The method of any one of the preceding embodiments, wherein prior to the freezing in (a), the population of cells is isolated and contacted with a poration solution.
20. The method of embodiments 19, wherein the poration solution comprises trehalose and wherein the concentration of trehalose in the poration solution is from about 0.1 M to about 1.0 M, or from about 0.1 M to about 0.6 M.
21. The method of any one of the preceding embodiments, wherein the population of cells comprises mammalian cells.
   Cryoprotection of Cells that are Stored by Freezing:

The present invention pertains to a method of producing a population of frozen cells, comprising freezing a composition comprising a population of cells, an aqueous component, urea, and a monosaccharide, to produce the population of frozen cells.

The present invention further pertains to a method of producing a population of reconstituted viable cells, comprising:
(a) freezing a composition comprising a population of cells, an aqueous component, urea, a monosaccharide, and optionally a bulking agent to produce a population of frozen cells; and
(b) resuspending the population of frozen cells in a reconstitution agent to form a reconstituted composition, wherein at least about 1% of the cells are viable.

In one embodiment, the concentration of the monosaccharide prior to freezing is from about 0.1 M to about 1.5 M. In one embodiment, the concentration of the monosaccharide prior to freezing is from about 0.2 M to about 1.2 M. Preferably, the concentration of the monosaccharide prior to freezing is from about 0.2 M to about 1.0 M. More preferably, the concentration of the monosaccharide prior to freezing is from about 0.2 M to about 0.8 M. Still more preferably, the concentration of the monosaccharide prior to freezing is from about 0.3 M to about 0.7 M. Even more preferably, the concentration of the monosaccharide prior to freezing is from about 0.4 M to about 0.6 M. Most preferably, the concentration of the monosaccharide prior to freezing is about 0.5 M.

In one embodiment, the concentration of the monosaccharide prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, 1.0 M, 1.05 M, 1.10 M, 1.15 M, 1.20 M, 1.25 M, 1.30 M, 1.35 M, 1.40 M, 1.45 M, or 1.50 M.

In one embodiment, the monosaccharide is glucose, fructose or galactose.

In one embodiment, the concentration of glucose prior to freezing is from about 0.1 M to about 1.5 M. In one embodiment, the concentration of glucose prior to freezing is from about 0.2 M to about 1.2 M. Preferably, the concentration of glucose prior to freezing is from about 0.2 M to about 1.0 M. More preferably, the concentration of glucose prior to freezing is from about 0.2 M to about 0.8 M. Still more preferably, the concentration of glucose prior to freezing is from about 0.3 M to about 0.7 M. Even more preferably, the concentration of glucose prior to freezing is from about 0.4 M to about 0.6 M. Most preferably, the concentration of glucose prior to freezing is about 0.5 M.

In one embodiment, the concentration of glucose prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, 1.0 M, 1.05 M, 1.10 M, 1.15 M, 1.20 M, 1.25 M, 1.30 M, 1.35 M, 1.40 M, 1.45 M, or 1.50 M.

In one embodiment, the concentration of urea prior to freezing is from about 0.1 M to about 1.5 M. In one embodiment, the concentration of urea prior to freezing is from about 0.2 M to about 1.2 M. Preferably, the concentration of urea prior to freezing is from about 0.2 M to about 1.0 M. More preferably, the concentration of urea prior to freezing is from about 0.2 M to about 0.8 M. Still more preferably, the concentration of urea prior to freezing is from about 0.2 M to about 0.8 M. Even more preferably, the concentration of urea prior to freezing is from about 0.4 M to about 0.6 M. Most preferably, the concentration of urea prior to freezing is about 0.5 M.

In one embodiment, the concentration of urea prior to freezing is about 0.1 M, 0.15 M, 0.2 M, 0.25 M, 0.3 M, 0.35 M, 0.4 M, 0.45 M, 0.5 M, 0.55 M, 0.6 M, 0.65 M, 0.7 M, 0.75 M, 0.8 M, 0.85 M, 0.9 M, 0.95 M, 1.0 M, 1.05 M, 1.10 M, 1.15 M, 1.20 M, 1.25 M, 1.30 M, 1.35 M, 1.40 M, 1.45 M, or 1.50 M.

In one embodiment, the molar ratio of urea to monosaccharide prior to freezing is from about 1:5 to about 5:1. In one embodiment, the molar ratio of urea to monosaccharide prior to freezing is from about 1:4 to about 4:1. In one embodiment, the molar ratio of urea to monosaccharide prior to freezing is from about 1:3 to about 3:1. In one embodiment, the molar ratio of urea to monosaccharide prior to freezing is from about 1:2 to about 2:1. Preferably, the molar ratio of urea to monosaccharide prior to freezing is about 1:1.

In one embodiment, the molar ratio of urea to glucose prior to freezing is from about 1:5 to about 5:1. In one embodiment, the molar ratio of urea to glucose prior to freezing is from about 1:4 to about 4:1. In one embodiment, the molar ratio of urea to glucose prior to freezing is from about 1:3 to about 3:1. In one embodiment, the molar ratio of urea to glucose prior to freezing is from about 1:2 to about 2:1. Preferably, the molar ratio of urea to glucose prior to freezing is about 1:1.

In one embodiment, the concentration of glucose prior to freezing is from about 0.2 M to about 1.2 M and the concentration of urea prior to freezing is from about 0.2 M to about 1.2 M. Preferably, the concentration of glucose prior to freezing is from about 0.2 M to about 1.0 M and the concentration of urea prior to freezing is from about 0.2 M to about 1.0 M. More preferably, the concentration of glucose prior to freezing is from about 0.2 M to about 0.8 M and the concentration of urea prior to freezing is from about 0.2 M to about 0.8 M. Even more preferably, the concentration of glucose prior to freezing is from about 0.4 M to about 0.6 M and the concentration of urea prior to freezing is from about 0.4 M to about 0.6 M. Most preferably, the concentration of glucose prior to freezing is about 0.5 M and the concentration of urea prior to freezing is about 0.5 M.

In one embodiment, the composition further comprises a bulking agent.

In one embodiment, the bulking agent is mannitol. In one embodiment, the concentration of mannitol prior to freezing is from about 0.1 M to about 1.0 M. In one embodiment, the concentration of mannitol prior to freezing is from about 0.1 M to about 0.8 M. Preferably, the concentration of mannitol prior to freezing is from about 0.1 M to about 0.6 M. More preferably, the concentration of mannitol prior to freezing is from about 0.15 M to about 0.55 M.

In one embodiment, the concentration of mannitol prior to freezing is from about 0.1 M to about 0.3 M. In one embodiment, the concentration of mannitol prior to freezing is from about 0.4 M to about 0.6 M.

In one embodiment, the bulking agent is sucrose. In one embodiment, the concentration of sucrose prior to freezing is from about 0.1 M to about 1.0 M. In one embodiment, the concentration of sucrose prior to freezing is from about 0.1 M to about 0.5 M. Preferably, the concentration of sucrose prior to freezing is from about 0.1 M to about 0.4 M. More preferably, the concentration of sucrose prior to freezing is from about 0.2 M to about 0.3 M.

In one embodiment, the composition comprises DMSO at a concentration of from about 1% to about 10% prior to freezing. In one embodiment, the composition comprises DMSO at a concentration of from about 1% to about 5% prior to freezing. In one embodiment, the composition comprises DMSO at a concentration of from about 1% to about 3% prior to freezing.

In one embodiment, prior to the freezing in (a), the population of cells is isolated and contacted with a poration solution. In one embodiment, the poration solution comprises trehalose and cell culture medium. In one embodiment, the concentration of trehalose in the poration solution is from about 0.1 M to about 1.0 M. In one embodiment, the concentration of trehalose in the poration solution is from about 0.1 M to about 0.8 M. In one embodiment, the concentration of trehalose in the poration solution is from about 0.1 M to about 0.6 M. In one embodiment, the concentration of trehalose in the poration solution is from about 0.1 M to about 0.3 M.

In one embodiment, the cells are contacted with the poration solution for at least 1 h, at least 2 h, at least 3 h, at least 4 h, at least 5 h, at least 6 h, at least 7 h, at least 8 h, at least 9 h, at least 10 h, at least 11 h, or at least 12 h. In one embodiment, the cells are contacted with the poration solution for 1 h to 24 h, for 1 h to 18 h, or for 1 h to 12 h.

In one embodiment, the composition comprises urea, glucose, and mannitol, wherein the concentration of glucose prior to freezing is from about 0.4 M to about 0.6 M, the concentration of urea prior to freezing is from about 0.4 M to about 0.6 M, and the concentration of mannitol prior to freezing is from about 0.1 M to about 0.6 M.

In one embodiment, the composition comprises urea, glucose, and sucrose, wherein the concentration of glucose prior to freezing is from about 0.4 M to about 0.6 M, the concentration of urea prior to freezing is from about 0.4 M to about 0.6 M, and the concentration of sucrose prior to freezing is from about 0.2 M to about 0.3 M.

In one embodiment, the composition comprises urea, glucose, sucrose, and DMSO wherein the concentration of glucose prior to freezing is from about 0.4 M to about 0.6 M, the concentration of urea prior to freezing is from about 0.4 M to about 0.6 M, the concentration of sucrose prior to freezing is from about 0.2 M to about 0.3 M, and the concentration of DMSO prior to freezing is from about 1% to about 3%.

The invention further pertains to a composition comprising a population of viable cells, an aqueous component, from about 0.2 M to about 1.0 M urea, from about 0.2 M to about 1.0 M glucose, and optionally a bulking agent.

In one embodiment, the composition comprises from about 0.3 M to about 0.8 M glucose, preferably from about 0.4 M to about 0.6 M glucose

In one embodiment, the composition comprises from about 0.3 M to about 0.8 M urea, preferably from about 0.4 M to about 0.6 M urea

In one embodiment, the bulking agent is sucrose. In one embodiment, the composition comprises from about 0.1 M to about 0.5 M. Preferably, the composition comprises from about 0.1 M to about 0.4 M. More preferably, the composition comprises from about 0.2 M to about 0.3 M.

In one embodiment, the bulking agent is mannitol. In one embodiment, the composition comprises from about 0.1 M to about 0.8 M. Preferably, the composition comprises from about 0.1 M to about 0.6 M. More preferably, the composition comprises from about 0.15 M to about 0.55 M.

In one embodiment, the composition further comprises DMSO. In one embodiment, the composition comprises from about 1% to about 10% DMSO. In one embodiment, the composition comprises from about 1% to about 5% DMSO. In one embodiment, the composition comprises from about 1% to about 3% DMSO.

Lyoprotection of Cells that are Stored by Lyophilization:

The present invention also pertains to a method of producing a population of lyophilized cells, comprising:
(a) freezing a composition comprising a population of cells, a hyaluronan gel, an aqueous component, urea, a monosaccharide, and a bulking agent, wherein the concentration of the monosaccharide prior to freezing is at least about 0.6 M, and the concentration of urea prior to freezing is at least about 0.6 M; and
(b) removing at least about 90% of water from the frozen composition to produce the population of lyophilized cells.

The present invention further pertains to a method of producing a population of reconstituted viable cells, comprising:
(a) freezing a composition comprising a population of cells, a hyaluronan gel, an aqueous component, urea, a monosaccharide, and a bulking agent, wherein the concentration of the monosaccharide prior to freezing is at least about 0.6 M, and the concentration of urea prior to freezing is at least about 0.6 M;
(b) removing at least about 90% of water from the frozen composition to produce the population of lyophilized cells, and
(c) resuspending the population of lyophilized cells in a reconstitution agent to form a reconstituted composition, wherein at least about 1% of the cells are viable.

Hyaluronan gel is known to the skilled artisan. In one embodiment, the hyaluronan gel is sourced from hyaluronic acid or hyaluronic acid sodium salt, preferably hyaluronic acid sodium salt. In one embodiment, the hyaluronan gel is based on PBS (phosphate buffered saline) or water and hyaluronic acid sodium salt. Preferably, the hyaluronan gel is based on PBS (phosphate buffered saline) and hyaluronic acid sodium salt.

In one embodiment, the hyaluronic acid or hyaluronic acid sodium salt is comprised in the composition in a concentration of about 5 mg/mL to about 50 mg/mL prior to freezing, such as at about 5 mg/mL, 10 mg/mL, 15 mg/mL, 20 mg/mL, 25 mg/mL, 30 mg/mL, 35 mg/mL, 40 mg/mL, 45 mg/mL, or 50 mg/mL. Preferably, the hyaluronic acid or hyaluronic acid sodium salt is comprised in the composition in a concentration of about 5 mg/mL to about 35 mg/mL prior to freezing, such as about 5 mg/mL to about 15 mg/mL, about 15 mg/mL to about 25 mg/mL, or about 25 mg/mL to about 35 mg/mL.

In one embodiment, the concentration of the monosaccharide prior to freezing is from about 0.6 M to about 2.0 M. In one embodiment, the concentration of the monosaccharide prior to freezing is from about 0.7 M to about 1.7 M. Preferably, the concentration of the monosaccharide prior to freezing is from about 0.8 M to about 1.4 M. More preferably, the concentration of the monosaccharide prior to freezing is from about 0.9 M to about 1.2 M. Most preferably, the concentration of the monosaccharide prior to freezing is about 1.0 M.

In one embodiment, the concentration of the monosaccharide prior to freezing is about 0.6 M, 0.65 M, 0.7 M, 0.75 M, 0.8 M, 0.85 M, 0.9 M, 0.95 M, 1.0 M, 1.05 M, 1.1 M, 1.15 M, 1.2 M, 1.25 M, 1.3 M, 1.35 M, 1.4 M, 1.45 M, 1.5 M, 1.55 M, 1.60 M, 1.65 M, 1.70 M, 1.75 M, 1.80 M, 1.85 M, 1.90 M, 1.95 M, or 2.0 M.

In one embodiment, the monosaccharide is glucose, fructose or galactose, preferably glucose.

In one embodiment, the concentration of glucose prior to freezing is from about 0.6 M to about 2.0 M. In one embodiment, the concentration of glucose prior to freezing is from about 0.7 M to about 1.7 M. Preferably, the concentration of glucose prior to freezing is from about 0.8 M to about 1.4 M. More preferably, the concentration of glucose prior to freezing is from about 0.9 M to about 1.2 M. Most preferably, the concentration of glucose prior to freezing is about 1.0 M.

In one embodiment, the concentration of glucose prior to freezing is about 0.6 M, 0.65 M, 0.7 M, 0.75 M, 0.8 M, 0.85 M, 0.9 M, 0.95 M, 1.0 M, 1.05 M, 1.1 M, 1.15 M, 1.2 M, 1.25 M, 1.3 M, 1.35 M, 1.4 M, 1.45 M, 1.5 M, 1.55 M, 1.60 M, 1.65 M, 1.70 M, 1.75 M, 1.80 M, 1.85 M, 1.90 M, 1.95 M, or 2.0 M.

In one embodiment, the concentration of urea prior to freezing is from about 0.6 M to about 2.0 M. In one embodiment, the concentration of urea prior to freezing is from about 0.7 M to about 1.7 M. Preferably, the concentration of urea prior to freezing is from about 0.8 M to about 1.4 M. More preferably, the concentration of urea prior to freezing is from about 0.9 M to about 1.2 M. Most preferably, the concentration of urea prior to freezing is about 1.0 M.

In one embodiment, the concentration of urea prior to freezing is about 0.6 M, 0.65 M, 0.7 M, 0.75 M, 0.8 M, 0.85 M, 0.9 M, 0.95 M, 1.0 M, 1.05 M, 1.1 M, 1.15 M, 1.2 M, 1.25 M, 1.3 M, 1.35 M, 1.4 M, 1.45 M, 1.5 M, 1.55 M, 1.60 M, 1.65 M, 1.70 M, 1.75 M, 1.80 M, 1.85 M, 1.90 M, 1.95 M, or 2.0 M.

In one embodiment, the molar ratio of urea to monosaccharide prior to freezing is from about 1:3 to about 3:1. In one embodiment, the molar ratio of urea to monosaccharide prior to freezing is from about 1:2 to about 2:1. Preferably, the molar ratio of urea to monosaccharide prior to freezing is about 1:1.

In one embodiment, the molar ratio of urea to glucose prior to freezing is from about 1:3 to about 3:1. In one embodiment, the molar ratio of urea to glucose prior to freezing is from about 1:2 to about 2:1. Preferably, the molar ratio of urea to glucose prior to freezing is about 1:1.

In one embodiment, the concentration of glucose prior to freezing is from about 0.6 M to about 2.0 M and the concentration of urea prior to freezing is from about 0.6 M to about 2.0 M. Preferably, the concentration of glucose prior to freezing is from about 0.7 M to about 1.7 M and the concentration of urea prior to freezing is from about 0.7 M to about 1.7 M. More preferably, the concentration of glucose prior to freezing is from about 0.8 M to about 1.4 M and the concentration of urea prior to freezing is from about 0.8 M to about 1.4 M. Even more preferably, the concentration of glucose prior to freezing is from about 0.9 M to about 1.2 M and the concentration of urea prior to freezing is from about 0.9 M to about 1.2 M. Most preferably, the concentration of glucose prior to freezing is about 1.0 M and the concentration of urea prior to freezing is about 1.0 M.

In one embodiment, the bulking agent is mannitol. In one embodiment, the concentration of mannitol prior to freezing is from about 0.1 M to about 1.0 M. In one embodiment, the concentration of mannitol prior to freezing is from about 0.1 M to about 0.8 M. Preferably, the concentration of mannitol prior to freezing is from about 0.1 M to about 0.6 M. More preferably, the concentration of mannitol prior to freezing is from about 0.1 M to about 0.4 M. In one embodiment, the concentration of mannitol prior to freezing is at least about 0.2 M.

In one embodiment, the bulking agent is sucrose. In one embodiment, the concentration of sucrose prior to freezing is from about 0.1 M to about 1.0 M. In one embodiment, the concentration of sucrose prior to freezing is from about 0.2 M to about 0.8 M. Preferably, the concentration of sucrose prior to freezing is from about 0.3 M to about 0.7 M. More preferably, the concentration of sucrose prior to freezing is from about 0.4 M to about 0.6 M. In one embodiment, the concentration of sucrose prior to freezing is at least about 0.4 M

In one embodiment, the composition comprises DMSO at a concentration of from about 1% to about 10% prior to freezing. In one embodiment, the composition comprises DMSO at a concentration of from about 1% to about 8% prior to freezing. In one embodiment, the composition comprises DMSO at a concentration of from about 2% to about 6% prior to freezing. In one embodiment, the composition comprises DMSO at a concentration of from about 3% to about 5% prior to freezing.

In one embodiment, prior to the freezing in (a), the population of cells is isolated and contacted with a poration solution. In one embodiment, the poration solution comprises trehalose and cell culture medium. In one embodiment, the concentration of trehalose in the poration solution is from about 0.1 M to about 1.0 M. In one embodiment, the concentration of trehalose in the poration solution is from about 0.1 M to about 0.8 M. In one embodiment, the concentration of trehalose in the poration solution is from about 0.1 M to about 0.6 M. In one embodiment, the concentration of trehalose in the poration solution is from about 0.1 M to about 0.3 M.

In one embodiment, the cells are contacted with the poration solution for at least 1 h, at least 2 h, at least 3 h, at least 4 h, at least 5 h, at least 6 h, at least 7 h, at least 8 h, at least 9 h, at least 10 h, at least 11 h, or at least 12 h. In one embodiment, the cells are contacted with the poration solution for 1 h to 24 h, for 1 h to 18 h, or for 1 h to 12 h.

In one embodiment, the composition comprises urea, glucose, sucrose and mannitol, wherein the concentration of glucose prior to freezing is from about 0.8 M to about 1.2 M, the concentration of urea prior to freezing is from about 0.8 M to about 1.2 M, the concentration of sucrose prior to freezing is from about 0.4 M to about 0.6 M, and the concentration of mannitol prior to freezing is from about 0.1 M to about 0.4 M.

In one embodiment, the composition comprises urea, glucose, sucrose, and DMSO, wherein the concentration of glucose prior to freezing is from about 0.8 M to about 1.2 M, the concentration of urea prior to freezing is from about 0.8 M to about 1.2 M, the concentration of sucrose prior to freezing is from about 0.4 M to about 0.6 M, and the concentration of DMSO prior to freezing is from about 3% to about 5%.

In one embodiment, the composition comprises urea, glucose, sucrose, mannitol and DMSO, wherein the concentration of glucose prior to freezing is from about 0.8 M to about 1.2 M, the concentration of urea prior to freezing is from about 0.8 M to about 1.2 M, the concentration of sucrose prior to freezing is from about 0.4 M to about 0.6 M, the concentration of mannitol prior to freezing is from about 0.1 M to about 0.4 M, and the concentration of DMSO prior to freezing is from about 3% to about 5%.

In one embodiment, the composition comprises urea, glucose, mannitol and DMSO, wherein the concentration of glucose prior to freezing is from about 0.8 M to about 1.2 M, the concentration of urea prior to freezing is from about 0.8 M to about 1.2 M, the concentration of mannitol prior to freezing is from about 0.1 M to about 0.4 M, and the concentration of DMSO prior to freezing is from about 3% to about 5%.

The present invention further pertains to a composition comprising a population of cells, a hyaluronan gel, an aqueous component, urea, a monosaccharide, and optionally a bulking agent, wherein urea and the monosaccharide are comprised in the composition at a molar ratio of about 1:3 to about 3:1, and wherein the composition contains less than about 10% (vol/vol) water.

In one embodiment, the molar ratio of urea to monosaccharide is from about 1:2 to about 2:1. Preferably, the molar ratio of urea to monosaccharide is about 1:1.

In one embodiment, the monosaccharide is glucose. In one embodiment, the molar ratio of urea to glucose prior to freezing is from about 1:2 to about 2:1. Preferably, the molar ratio of urea to glucose prior to freezing is about 1:1.

In one embodiment, the composition contains less than about 5% (vol/vol) water.

### EXAMPLES

### Example 1—Cryoprotection and Lyoprotection of Human Mesenchymal Stem Cells

### SUMMARY

Human mesenchymal stem cells (hMSCs) are capable of in vitro differentiation into various types of tissues, such as bone, fat, cartilage or muscle, which renders them suitable for use in research and cell therapy products. However, current storage methods for hMSCs in liquid nitrogen suffer from drawbacks. Storage and distribution are costly and labor intensive, and standard freezing protocols commonly employ agents such as fetal bovine serum (FBS) and dimethyl sulfoxide (DMSO), which can cause aversive immune reactions or are toxic, respectively. Thus, these agents need to be removed before a product containing frozen hMSCs is applied to humans. Lyophilization of cells in the non-toxic formulation would be a potential solution, but, as of today, there are no satisfying non-toxic formulations with cryoprotective and lyoprotective properties that have been reported.

The work presented herein provides the first known evaluation of the cryoprotective capabilities of the non-toxic and synergistically working cryoprotective agents (CPAs) urea and glucose with hMSCs. Different lyophilization strategies using urea/glucose as CPAs are qualitatively evaluated herein. Freezing of hMSCs at −80° C. in 0.5 M urea and 0.5 M glucose gained comparable viabilities to that of the DMSO control. Also, hMSCs in formulations with high amounts of CPAs were able to withstand the harsh conditions of a lyophilization and they were proliferated upon thawing.

### INTRODUCTION

Human mesenchymal stem cells (hMSCs) are important pluripotent stem cells with the ability to differentiate (in vitro) into different tissues such as fat, muscle, cartilage, bone, or neural cells. The use of hMSCs in regenerative medicine and cell therapy is expanding. As hMSCs exhibit immunosuppressive capabilities they have the potential to be harvested from an adult donor, expanded, modified, and subsequently used in allogenic treatments. To enable this, hMSCs have to be stored for long periods of time. The current protocol for extended storage of hMSCs is by cryopreservation in liquid nitrogen. However, storing cells in liquid nitrogen is cumbersome and expensive, especially for distribution and transport, as a regular supply of liquid nitrogen is needed to avoid uncontrolled thawing and damage to the cells.

Liquid nitrogen Storage of hMSCs also requires the addition of fetal bovine serum (FBS) and dimethyl sulfoxide (DMSO) to preserve the cells. DMSO is toxic and can alter the expression of transcription factors and gene expression in hMSCs. Moreover, FBS is highly immunogenic in humans and could transfer pathogens. Hence, DMSO and FBS are normally removed prior to administration to humans, a process which is labor intense and requiring specially trained personnel and special facilities. There are therefore safety implications with a high risk of a sub-standard and inconsistent quality of the administered product.

It would also be beneficial to store a cell therapy product (CTP) at refrigerated temperatures similar to many biological products to facilitate easier handling and economical shipping. One possible way to achieve this would be to lyophilize (freeze-dry) the cells. Lyophilization is routinely applied to (drug) products that are unstable in liquid formulations, but stabilized in solid/dried form, such as vaccines, proteins, peptides or antibiotics. Applying the principle of lyophilization to hMSCs would greatly support their utility as CTP.

To cryopreserve hMSCs, various alternatives to DMSO have been explored: Predominately the intra- or extracellular introduction of sugars or sugar alcohols such as sucrose, trehalose, glucose, mannitol, glycerol, or ethylene glycol, but also polyvinylpyrrolidon, pentaisomaltose, or ectoin have been evaluated.

In some species of hibernating frogs, accumulation of urea is reported to have osmoprotectitve and cryoprotective effects. These effects were further strengthened by additional increase of somatic glucose levels and glucose/urea combinations improved the tolerances to the freezing stress. As these agents are relatively safe in humans, cheap, and commercially available, the potential of urea/glucose combinations for cryoprotection and lyophilization in hMSCs was explored by means of advanced formulations, hydrogels, and stabilizers.

### Materials and Methods

### Cell Culture

Adherent bone marrow derived human mesenchymal stem cells (hMSCs) (ATCC) were expanded to create a cell bank and stored in the vapor phase of liquid nitrogen in growth medium supplemented with 50% FBS and 7.5% DMSO at passage 3. Aliquots of those samples were warmed in a 37° C. water bath and cultured in MSCGM™ Mesenchymal Stem Cell Growth Medium BulletKit™ (containing FBS, Lonza); a commercially available medium that facilitates undifferentiated proliferation of hMSCs. The cells were kept in a humidified incubator at 37° C. and 5% CO₂, whilst the growth medium was changed every 3-4 days. When hMSCs reached confluence of ˜90%, they were split and re-seeded at ˜5000 cells/cm² to facilitate further expansion. For experiments, cells between passages 4-8 were used.

### Freeze-Thaw Experiments

Confluent or near confluent hMSCs were harvested by aspirating the medium, washing with pre-warmed phosphate buffered saline, pH 7.4 (PBS, Gibco) and detaching the cells by applying ˜0.02 ml per cm² trypsin solution (Lonza). Afterwards, the trypsin reaction was stopped by adding growth medium. An aliquot was taken for determination of cell concentration (using the NucleCounter® NC-200™). Cells were then pelleted by centrifugation and subsequently re-suspended in PBS to achieve a target concentration of 2×10⁶ cells per ml. A volume of 0.5 ml cell suspension was added to a 6R vial (Schott) containing 0.5 ml of a 2-fold concentrated solution of cryoprotective agents (CPAs) (urea: GE Healthcare, glucose: Aldrich, sucrose and trehalose: Pfanstiehl, mannitol: Avantor, DMSO: Arcos) diluted in PBS, resulting in a total volume of 1 ml containing 1×10⁶ cells and the 1-fold target concentration of cryoprotectant(s). Subsequently, vials were stoppered (West) and transferred to a −80° C. freezer where they were stored for 2-3 days until viability measurements were conducted. Each experiment was repeated five times.

### Freeze-Drying Experiments

hMSCs were either cultured on different hydrogels (HyStem™, HyStem™ C, or agar (Sigma), or suspended in hyaluronic acid (HA) (Alfa Aesar), to provide a gel matrix that can be used for culturing and/or as a scaffold for a lyo cake.

Samples for Freeze-Drying Experiments were Prepared in the Following Way:

Agarose gels were prepared by dissolving 2% agar in PBS while heating. The gels were cooled and jellified for about 20 h. Subsequently, cells were seeded on the gel's surfaces, with a concentration of about 20,000 cells per square centimeter gel, and incubated with cell culture medium for about 20 h, so that the cells could re-attach to the gels surface. Before lyophilization, the medium was removed and a layer of a viscous solution containing HA and 1-fold concentrations of CPAs (so called viscous CPA/HA formulations, as seen in Table 1, they were prepared by dissolving the appropriate 1-fold concentration of CPAs in PBS and subsequently adding hyaluronic acid sodium salt, the solutions were stirred for about 20 h to ensure full solution of HA (when HA is dissolved, the solution became viscous) to provide the viscous CPA/HA formulations) was added on top of the agarose gel/cell layer and samples were frozen at −80° C. for about 20 h before being lyophilized.

HyStem™ gels were prepared according to the manufacturer's instructions. On top of these HyStem™ gels cell were seeded on the gel's surface then overlayed with the viscous CPA/HA formulations as described for the preparation of the agarose gels.

The viscous CPA/HA formulation was prepared by dissolving the appropriate 1-fold concentration of CPAs in PBS and subsequently adding hyaluronic acid sodium salt to arrive at the target concentration of 10 mg/ml HA salt (=1% w/v). The solutions were stirred for about 20 h to ensure full solution of HA (when HA is dissolved, the solution became viscous) to provide viscous CPA/HA formulations.

For cells in suspension with HA, the viscous CPA/HA formulations were prepared as described. The cells were prepared in analogy to the preparation of the cells described for the freeze-thaw experiments: after the stopping of the trypsin reaction by adding growth medium, cells were pelleted by centrifugation and subsequently re-suspended in PBS to achieve a target concentration of 2×10⁶ cells per ml. A volume of 0.5 ml cell suspension was added to a 6R vial (Schott) and then, instead of layering cells on top of a gel, they were spun down in the 6R vial and then resuspended in 1 ml of the viscous CPA/HA formulation.

After the addition of the CPA solution or of the viscous CPA/HA formulations, all vials were immediately moved to a −80° C. freezer where they were frozen overnight. For lyophilization, samples were put into a pilot scale LyoStar™ 3 freeze-dryer that was pre-cooled to ˜65° C. The samples where lyophilized for either 1,000 (cycle 1) or 1,800 (cycle 2) or 3,640 (cycle 3) minutes at −40° C. at <75 mTorr. A secondary drying step was omitted in order to avoid potential loss of viable cells. After lyophilization, the samples were reconstituted in 6 ml of warm growth medium and transferred to a flask to allow the cells to re-attach to the plastic surface.

After lyophilization the composition still contains a certain amount of water, typically less than about 90% (vol/vol) water.

### Viability Assessment

For frozen samples, that is for samples after freezing and thawing, viability was measured by performing an alamarBlue™ assay (Invitrogen) according to the manufacturer's instructions. The assay is based on the reduction of a non-fluorescent dye into a fluorescent one. This reaction only takes place in cells that are metabolically active (viable), thus correlating measured fluorescence with viable cells. Frozen cells were thawed in a 37° C. water bath and plated in a 48-well plate at densities of 20000 cells per cm² (dead and alive cells) and kept in the incubator overnight, in order to allow viable cells time to attach to the surface and to re-gain their metabolic activity. Assay reagent was then applied and, after a 4 h incubation, fluorescence was measured using a SpectraMax® iD3 with wavelengths of λₑₓ=550 nm and λₑₘ=590 nm. Background fluorescence values were subtracted from the measurements and compared to fluorescence values of untreated cells.

For lyophilized samples, hMSCs were qualitatively evaluated for viability by reconstituting the sample in growth medium and re-seeding the entire content of the vial in a flask. After overnight incubation, cells were microscopically inspected so see whether they re-attached to the surface of the flask and whether the morphology showed no irregularities compared to the known morphology of viable cells. Cells were subsequently cultured as previously mentioned and inspected for proliferation after several days. For all experiments the sample size is one per condition.

### Results and Discussion

Urea and Glucose as Cryoprotectants in hMSCs in Freeze-Thawing

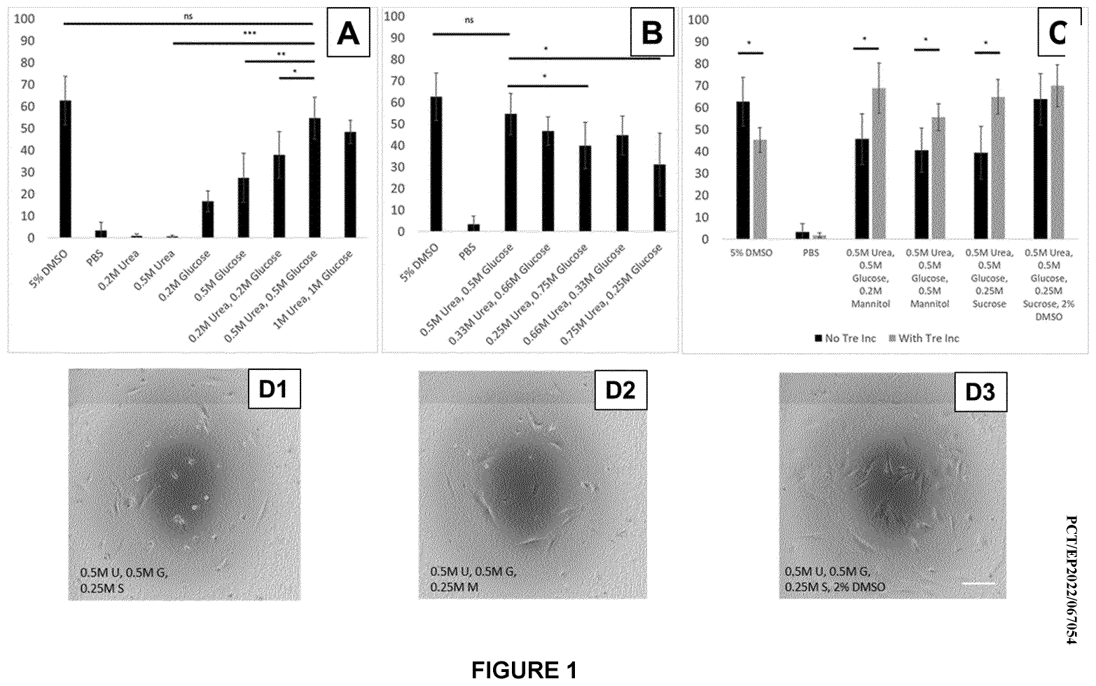

Different concentrations of urea and glucose were assessed for the cryoprotection of hMSCs (FIG. 1A). Concentrations of 0.05, 0.1 (these two are not shown in FIG. 1A), 0.2, and 0.5 M urea had virtually no viable cells, showing that urea alone is not sufficient to protect hMSCs during cryopreservation. Interestingly, about 26% cell viability was achieved in 0.5 M of glucose alone, which slightly reduced to about 20% when glucose concentration was lowered to 0.2 M. A combination of urea with glucose (0.5 M glucose and 0.5 M urea), further increased the number of viable cells to comparable levels of the 5% DMSO control (58%). A further increase of urea/glucose to 1 M each decreased the viability of hMSCs to about 50%. These results suggest that in hMSCs urea and glucose work in synergy and that their combined cryoprotective capacities are greater than the ones of each agent alone. As shown in FIG. 1A, combinations of 0.5 M urea and 0.5 M glucose are optimal in protecting hMSCs from freeze/thaw (FT) stress and cell viability was on par with control. Further assessment of different ratios of urea to glucose to potentially increase FT survival rates (FIG. 1B) confirmed that the 1:1 molar ratio of urea/glucose at 0.5 M provides maximal cryoprotection. The following cell viabilities were observed: urea:glucose molar ratios: 1:2 ratio: 45%, 1:3 ratio: 40%. 2:1 ratio: 45%, 3:1 ratio: 30%. Of the tested ratios, none had larger viabilities than the optimal 1:1 molar ratio.

### Formulation for Lyophilization Tested by Freeze-Thawing

Since cryopreservation was successfully achieved using a 1:1 molar ratio of urea/glucose at concentrations of 0.5 M, it was attempted to improve the formulation for the primary drying step of the lyophilization process. Sucrose and mannitol were selected as bulking agents for their cake formation capabilities with favorable lyoprotectant functionality, as sucrose can improve post-FT viability in stem cells.

The addition of 0.2 M and 0.5 M mannitol provided 45% and 40% viable cells, respectively, and 0.25 M of sucrose provided 39% viable cells (FIG. 1C). Although cell viabilities are lower than the previous FT control or formulations with 0.5 M urea/glucose, viability rates were further improved by addition of small amounts of DMSO (2%) to the urea/glucose/sucrose formulation. The drop in the viability rates with increasing concentrations of sugars is possibly due to an increase in osmotic stress. Sugar concentrations with high viability appear to capture the benefits of high molar amounts of extracellular sugars as CPAs without causing too great an increase in osmotic pressure. It is possible that internalization and intracellular accumulation of sugars is beneficial for FT survival of hMSCs

### Internalization of Trehalose as Cryo- and Lyoprotective Agent

Trehalose is known to act as a cryo- and lyoprotectant, but can only when it is present both, intra- and extracellularly. However the molecule is not membrane permeable. Some methods for intracellular trehalose incorporation thus use genetic modification of cellular pores, cell penetrating peptides, liposomes or electroporation, however these methods are complex, costly, time consuming, and may cause significant alterations to the genetic make-up of the hMSCs. An alternative, simple method of internalization involves trehalose uptake by endocytosis from the media supplemented with the trehalose, which is reported to act as a CPA [32].

The for about 20 h incubation of hMSCs with 0.2 M trehalose dissolved in cell culture medium and subsequent FT in the optimal formulation of 0.5 M urea/0.5 M glucose/0.25 M sucrose showed good viability without morphological alterations such as shrinkage, suggesting hMSCs could withstand the applied osmotic stress (FIG. 1C and FIGS. 1D1-1D3). An additional incubation in trehalose slightly boosted the survival rate of hMSCs in the urea/glucose/sucrose formulation to about 65% and was able to be increased further to about 72% with addition of 2% DMSO, suggesting that the combination of different CPAs is superior to single CPA to improve cell survival on FT. Cell viability was about 70% with 0.2 M mannitol formulation. These results demonstrate that hMSCs can withstand the short term hypertonic conditions in growth medium supplemented with 0.2 M trehalose and it further helps to improve cryopreservation and potentially lyopreservation of hMSCs.

Lyophilization of hMSCs

### Lyophilization Cycle Optimization

The lyo cycle and residual moisture might have a significant impact on hMSC viability. Two different drying times were evaluated at −40° C.: 1,000 to 1,800 to 3,640 minutes. Steps were also taken to further optimize the loading and unloading temperatures and reconstitution procedure.

### Lyophilization Using 3D Gel Matrices

Different lyophilization strategies were evaluated for hMSCs using urea and glucose as primary CPAs, as well as sucrose, mannitol, and trehalose. hMSCs were allowed to grow on different gel matrices, namely, HyStem™, HyStem™ C, and 2% agarose gel. HyStem™ and HyStem™ C are commercially available gels for 3D cell cultures based on thiol modified hyaluronic acid; HyStem™ C additionally contains collagen fibers serving as attachment sites and thus improves cell adherence to the gel [33]. Alternatively, hMSCs were suspended and subsequently lyophilized in hyaluronic acid (HA) and CPAs.

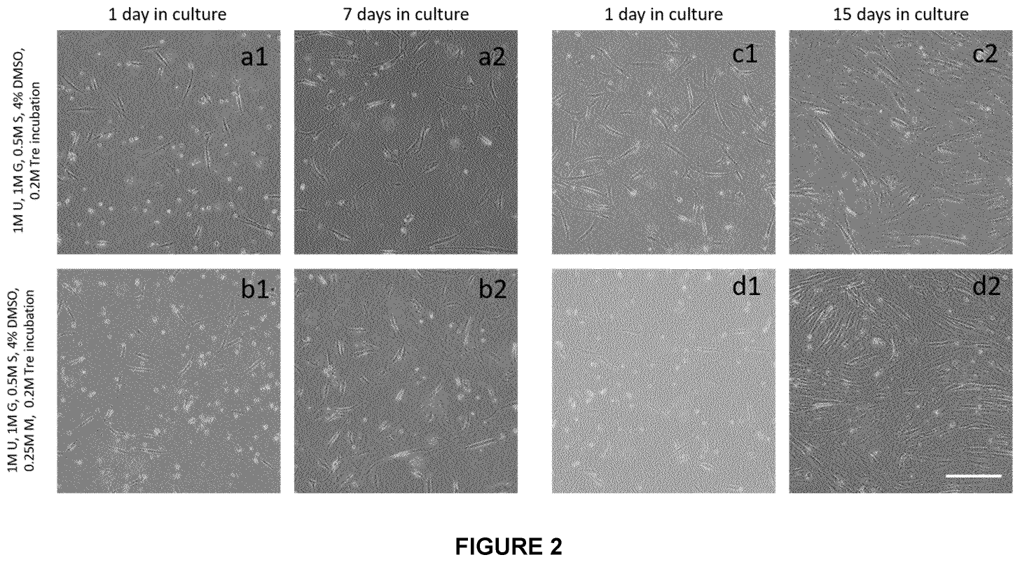

hMSCs cultured on HyStem™ had no viable cells after lyophilization, independent of which CPAs were used (Table 1). The cells were also not able to attach the hydrogel surface and form a layer of cells. Cells cultured on HyStem™ C matrix were able to attach to the gel, but also did not show significant cell viability after the lyophilization. Also, cell viability remained very low in Agarose/HA dual matrix. However, hMSCs suspended in a viscous formulation of HA and different amounts of CPAs at higher concentrations were able to obtain viable cells that attached to the flask and also proliferated (FIG. 2) after the lyophilization, indicating hMSCs are viable and can withstand the harsh condition of the lyophilization process. To the knowledge of the inventors, this is first instance to report lyophilization of unmodified hMSCs with unaltered morphology, and to make use of the combination of urea and glucose in such a process. hMSCs also remained viable in HA gel with optimal CPA amounts, but without intracellular trehalose incubation or addition of DMSO (Table 1). Although cells survived the lyophilization process, the cake formation was subpar with high-level of visible liquid droplets, suggesting partial melt-back. The cake formation and quality did not increase by further increasing the amount of HA and/or addition of mannitol, but cells remained viable.

TABLE 1
Qualitative viability results of lyophilized hMSCs with different CPAs and parameters.

| | | Concentration | | | | | |
|---|---|---|---|---|---|---|---|
| | Gel | of HA | | Lyo | Trehalose | Cake | Viable |
| No. | matrix | [mg/ml] | CPAs | cyle | Incubation | Formation | cells? |
| 1 | 1 | n/a | 5% DMSO | Cycle 1 | No | No | No |
| 2 | 1 | n/a | 0.25M U, 0.75M G | Cycle 1 | No | No | No |
| 3 | 1 | n/a | 0.5M U, 0.5M G | Cycle 1 | No | No | No |
| 4 | 1 | n/a | 0.5M U, 0.5M G, 0.25M S | Cycle 1 | No | No | No |
| 5 | 1 | n/a | 0.33M U, 0.33M G, 0.16M S, | Cycle 1 | No | No | No |
| | | | 3% DMSO | | | | |
| 6 | 2 | 10 | 0.5M U, 0.5M G | Cycle 1 | Yes | No | No |
| 7 | 2 | 10 | 0.5M U, 0.2M G, 0.5M M | Cycle 1 | Yes | No | No |
| 8 | 2 | 10 | 1M U, 1M G, 0.5M S, | Cycle 1 | Yes | No | No |
| | | | 4% DMSO | | | | |
| 9 | 2 | 10 | 0.5M U, 0.2M G, 0.2M | Cycle 1 | Yes | No | No |
| 10 | 2 | 10 | 0.5M U, 0.5M G | Cycle 1 | No | No | No |
| 11 | 2 | 10 | 0.5M U, 0.2M G, 0.5M M | Cycle 1 | No | No | No |
| 12 | 2 | 10 | 1M U, 1M G, 0.5M S, | Cycle 1 | No | No | No |
| | | | 4% DMSO | | | | |
| 13 | 2 | 10 | 0.5M U, 0.2M G, 0.2M | Cycle 1 | No | No | No |
| 14 | 3 | 10 | 5% DMSO | Cycle 1 | No | No | No |
| 15 | 3 | 10 | 0.5M U, 0.5M G | Cycle 1 | No | No | No |
| 16 | 3 | 10 | 0.5M U, 0.2M G, 0.5M M | Cycle 1 | No | No | No |
| 17 | 3 | 10 | 1M U, 1M G, 0.5M S, | Cycle 1 | No | No | No |
| | | | 4% DMSO | | | | |
| 18 | 3 | 10 | 0.5M U, 0.2M G, 0.2M | Cycle 1 | No | No | No |
| 19 | 4 | 10 | 5% DMSO | Cycle 1 | No | No | No |
| 20 | 4 | 10 | 0.5M U, 0.5M G | Cycle 1 | No | No | No |
| 21 | 4 | 10 | 0.5M U, 0.2M G, 0.5M M | Cycle 1 | No | Yes | No |
| 22 | 4 | 10 | 1M U, 1M G, 0.5M S, | Cycle 1 | No | No | Yes |
| | | | 4% DMSO | | | | |
| 23 | 4 | 10 | 0.5M U, 0.2M G, 0.2M | Cycle 1 | No | No | No |
| | | | trehalose | | | | |
| 24 | 4 | 10 | 0.5M U, 0.5M G, 0.25M S, | Cycle 1 | No | No | No |
| | | | 2% DMSO | | | | |
| 25 | 4 | 10 | 0.5M U, 0.5M G, 0.25M S, | Cycle 1 | Yes | No | No |
| | | | 2% DMSO | | | | |
| 26 | 4 | 10 | 1M U, 1M G, 0.5M S, | Cycle 1 | No | No | Yes |
| | | | 4% DMSO | | | | |
| 27 | 4 | 10 | 1M U, 1M G, 0.5M S, | Cycle 1 | Yes | No | Yes |
| | | | 4% DMSO | | | | |
| 28 | 4 | 10 | 1M U, 1M G, 0.5M S, | Cycle 1 | Yes | No | Yes |
| | | | 4% DMSO | | | | |
| 29 | 4 | 20 | 1M U, 1M G, 0.5M S, | Cycle 1 | Yes | No | Yes |
| | | | 4% DMSO | | | | |
| 30 | 4 | 30 | 1M U, 1M G, 0.5M S, | Cycle 1 | Yes | No | Yes |
| | | | 4% DMSO | | | | |
| 31 | 4 | 10 | 1M U, 1M G, 0.5M S, | Cycle 1 | Yes | No | Yes |
| | | | 4% DMSO, 0.25M M | | | | |
| 32 | 4 | 20 | 1M U, 1M G, 0.5M S, | Cycle 1 | Yes | No | Yes |
| | | | 4% DMSO, 0.25M M | | | | |
| 33 | 4 | 30 | 1M U, 1M G, 0.5M S, | Cycle 1 | Yes | No | Yes |
| | | | 4% DMSO, 0.25M M | | | | |
| 34 | 4 | 10 | 1M U, 1M G, 4% DMSO, | Cycle 1 | Yes | No | Yes |
| | | | 0.25M M | | | | |
| 35 | 4 | 20 | 1M U, 1M G, 4% DMSO, | Cycle 1 | Yes | No | Yes |
| | | | 0.25M M | | | | |
| 36 | 4 | 30 | 1M U, 1M G, 4% DMSO, | Cycle 1 | Yes | No | Yes |
| | | | 0.25M M | | | | |
| 37 | 4 | 10 | 1M U, 1M G, 0.5M S, | Cycle 2 | Yes | No | Yes |
| | | | 4% DMSO | | | | |
| 38 | 4 | 20 | 1M U, 1M G, 0.5M S, | Cycle 2 | Yes | No | Yes |
| | | | 4% DMSO | | | | |
| 39 | 4 | 10 | 1M U, 1M G, 0.5M S, | Cycle 2 | Yes | No | Yes |
| | | | 4% DMSO, 0.25M M | | | | |
| 40 | 4 | 20 | 1M U, 1M G, 0.5M S, | Cycle 2 | Yes | No | Yes |
| | | | 4% DMSO, 0.25M M | | | | |
| 41 | 4 | 10 | 1M U, 1M G, 4% DMSO, | Cycle 2 | Yes | No | Yes |
| | | | 0.25M M | | | | |
| 42 | 4 | 20 | 1M U, 1M G, 4% DMSO, | Cycle 2 | Yes | No | No |
| | | | 0.25M M | | | | |
| 43 | 4 | 10 | 1M U, 1M G, 0.5M S, | Cycle 2 | Yes | No | Yes |
| | | | 0.25M M | | | | |
| 44 | 4 | 20 | 1M U, 1M G, 0.5M S, | Cycle 2 | Yes | No | Yes |
| | | | 0.25M M | | | | |
| 45 | 4 | 10 | 0.5M U, 0.5M G, 0.25M S, | Cycle 2 | Yes | Yes | No |
| | | | 0.25M M | | | | |
| 46 | 4 | 20 | 0.5M U, 0.5M G, 0.25M S, | Cycle 2 | Yes | Yes | No |
| | | | 0.25M M | | | | |
| 47 | — | 0 | 1M U, 1M G, 0.5M S, | Cycle 2 | Yes | No | Yes |
| | | | 4% DMSO | | | | |

U = urea, G = glucose, DMSO = dimethyl sulfoxide, M = mannitol, S = sucrose, HA = hyaluronic acid. Gel matrices: (1) HyStem ™ and HA, (2) HyStem ™C and HA, (3) agar and HA, (4) hyaluronan gel. Lyo cycles ran for 1000 (cycle 1) or 1800 (cycle 2) minutes at −40° C. at <75 mTorr.
Examples 22 and 26 are repetitions, as well as Examples 27 and 28.
Examples 43 and 44 show lyophilization experiments without DMSO which provided viable cells.

### CONCLUSION

Human mesenchymal stem cell CTPs still employ toxic and/or immune response eliciting agents (DMSO and FBS) for storage of cells. Safe and effective alternative CPAs to DMSO were identified for cryopreservation. Urea and glucose exhibited synergistic CPA activity at equimolar concentrations in cryopreservation of hMSCs. Further addition of sugars and optimization of formulation helped in improving the cryopreservation. Attempts were made to improve cell viability by optimizing formulation and lyophilization processes. Early attempts of lyophilization showed hMSC survival and proliferation capabilities. Even for lyophilization examples were found which provided viable cells after lyophilization without DMSO.

### REFERENCES

1. Barry, F. P. and J. M. Murphy, Mesenchymal stem cells: clinical applications and biological characterization. Int J Biochem Cell Biol, 2004. 36(4): p. 568-84.
2. Ullah, I., R. B. Subbarao, and G. J. Rho, Human mesenchymal stem cells—current trends and future prospective. Biosci Rep, 2015. 35(2).
3. Pittenger, M. F., et al., Multilineage potential of adult human mesenchymal stem cells. Science, 1999. 284(5411): p. 143-7.
4. Nakamizo, A., et al., Human bone marrow-derived mesenchymal stem cells in the treatment of gliomas. Cancer Res, 2005. 65(8): p. 3307-18.
5. Yong, K. W., et al., Cryopreservation of Human Mesenchymal Stem Cells for Clinical Applications: Current Methods and Challenges. Biopreserv Biobank, 2015. 13(4): p. 231-9.
6. De Miguel, M. P., et al., Immunosuppressive properties of mesenchymal stem cells: advances and applications. Curr Mol Med, 2012. 12(5): p. 574-91.
7. Yong, K. W., J. R. Choi, and W. K. Z. Wan Safwani, Biobanking of Human Mesenchymal Stem Cells: Future Strategy to Facilitate Clinical Applications, in Biobanking and Cryopreservation of Stem Cells, F. Karimi-Busheri and M. Weinfeld, Editors. 2016, Springer International Publishing: Cham. p. 99-110.
8. Arav, A. and J. Saragusty, Directional freezing of sperm and associated derived technologies. Anim Reprod Sci, 2016. 169: p. 6-13.
9. Germann, A., et al., Temperature fluctuations during deep temperature cryopreservation reduce PBMC recovery, viability and T-cell function. Cryobiology, 2013. 67(2): p. 193-200.
10. Stewart, S. and X. He, Intracellular Delivery of Trehalose for Cell Banking. Langmuir, 2019. 35(23): p. 7414-7422.
11. Galvao, J., et al., Unexpected low-dose toxicity of the universal solvent DMSO. FASEB J, 2014. 28(3): p. 1317-30.
12. Hanslick, J. L., et al., Dimethyl sulfoxide (DMSO) produces widespread apoptosis in the developing central nervous system. Neurobiol Dis, 2009. 34(1): p. 1-10.
13. Martino, M., et al., Fractionated infusions of cryopreserved stem cells may prevent DMSO-induced major cardiac complications in graft recipients. Haematologica, 1996. 81(1): p. 59-61.
14. Ruiz-Delgado, G. J., et al., Dimethyl sulfoxide-induced toxicity in cord blood stem cell transplantation: report of three cases and review of the literature. Acta Haematol, 2009. 122(1): p. 1-5.
15. Davies, O. G., et al., The effects of cryopreservation on cells isolated from adipose, bone marrow and dental pulp tissues. Cryobiology, 2014. 69(2): p. 342-7.
16. Woodbury, D., et al., Adult rat and human bone marrow stromal cells differentiate into neurons. J Neurosci Res, 2000. 61(4): p. 364-70.
17. Thirumala, S., et al., Evaluation of polyvinylpyrrolidone as a cryoprotectant for adipose tissue-derived adult stem cells. Tissue Eng Part C Methods, 2010. 16(4): p. 783-92.
18. Oliver, A. E., Dry state preservation of nucleated cells: progress and challenges. Biopreserv Biobank, 2012. 10(4): p. 376-85.
19. Weng, L., Technologies and Applications Toward Preservation of Cells in a Dry State for Therapies. Biopreserv Biobank, 2021.
20. Izutsu, K. i., Applications of Freezing and Freeze-Drying in Pharmaceutical Formulations, in Survival Strategies in Extreme Cold and Desiccation: Adaptation Mechanisms and Their Applications, M. Iwaya-Inoue, M. Sakurai, and M. Uemura, Editors. 2018, Springer Singapore: Singapore. p. 371-383.
21. Weng, L. and P. R. Beauchesne, Dimethyl sulfoxide-free cryopreservation for cell therapy: A review. Cryobiology, 2020. 94: p. 9-17.
22. Gomez-Fernandez, J., et al., Effect of different monosaccharides and disaccharides on boar sperm quality after cryopreservation. Anim Reprod Sci, 2012. 133(1-2): p. 109-16.
23. Pollock, K., et al., Combinations of Osmolytes, Including Monosaccharides, Disaccharides, and Sugar Alcohols Act in Concert During Cryopreservation to Improve Mesenchymal Stromal Cell Survival. Tissue Eng Part C Methods, 2016. 22(11): p. 999-1008.
24. Freimark, D., et al., Systematic parameter optimization of a Me(2)SO-and serum-free cryopreservation protocol for human mesenchymal stem cells. Cryobiology, 2011. 63(2): p. 67-75.
25. Svalgaard, J. D., et al., Low-molecular-weight carbohydrate Pentaisomaltose may replace dimethyl sulfoxide as a safer cryoprotectant for cryopreservation of peripheral blood stem cells. Transfusion, 2016. 56(5): p. 1088-95.
26. Costanzo, J. P. and R. E. Lee, Jr., Urea loading enhances freezing survival and post freeze recovery in a terrestrially hibernating frog. J Exp Biol, 2008. 211(Pt 18): p. 2969-75.
27. Costanzo, J. P. and R. E. Lee, Jr., Cryoprotection by urea in a terrestrially hibernating frog. J Exp Biol, 2005. 208(Pt 21): p. 4079-89.
28. Pan, M., et al., Urea: a comprehensive review of the clinical literature. Dermatol Online J, 2013. 19(11): p. 20392.
29. Janz Fde, L., et al., Evaluation of distinct freezing methods and cryoprotectants for human amniotic fluid stem cells cryopreservation. J Biomed Biotechnol, 2012. 2012: p. 649353.
30. Bissoyi, A., et al., Recent Advances and Future Direction in Lyophilisation and Desiccation of Mesenchymal Stem Cells. Stem Cells Int, 2016. 2016: p. 3604203.
31. Oliver, A. E., et al., Loading Human Mesenchymal Stem Cells with Trehalose by Fluid-Phase Endocytosis. Cell Preservation Technology, 2004. 2: p. 35-49.
32. Zhang, S. Z., et al., Preliminary study on the freeze-drying of human bone marrow-derived mesenchymal stem cells. J Zhejiang Univ Sci B, 2010. 11(11): p. 889-94.
33. HyStemC Product Information. 2021 [cited 2021 22 Apr. 2021]; Available from: https://www.sigmaaldrich.com/content/dam/sigma-aldrich/docs/Sigma/Bulletin/hysc020bul.pdf.

1. A composition comprising a population of viable cells, an aqueous component, urea and a monosaccharide, wherein the composition further comprises a hyaluronan gel and wherein the concentration of the monosaccharide is from about 0.4 M to about 2.0 M.

2. The composition according to claim 1, wherein the composition further comprises a bulking agent.

3. (canceled)

4. The composition according to claim 1, wherein the concentration of the monosaccharide is from about 0.6 M to about 2.0 M, or from about 0.7 M to about 1.7 M, or from about 0.8 M to about 1.4 M, or from about 0.4 M to about 1.25 M, or from about 0.4 M to about 0.8 M, or from about 0.4 M to about 0.6 M.

5. The composition according to claim 1, wherein the concentration of the urea is from about 0.7 M to about 1.7 M, or from about 0.8 M to about 1.4 M, or from about 0.2 M to about 1.25 M, or from about 0.2 M to about 0.8 M, or from about 0.4 M to about 0.6 M, or from about 0.6 M to about 2.0 M.

6. The composition according to claim 1, wherein the molar ratio of the urea to monosaccharide in the composition being from about 5:1 to about 1:5, or from about 1:3 to about 3:1, or from about 1:2 to about 2:1, or is about 1:1.

7. The composition according to claim 1, wherein the monosaccharide is glucose, fructose or galactose.

8. The composition according to claim 1, wherein the bulking agent comprises
a) a disaccharide,
wherein the disaccharide is sucrose, lactose, maltose, trehalose, cellobiose, chitobiose, lactulose, isomaltose melibiose or gentiobiose; or
b) a sugar alcohol,
wherein the sugar alcohol is mannitol, sorbitol, galactitol, fucitol, iditol or inositol; or
c) both a disaccharide and a sugar alcohol; or
d) both sucrose and mannitol.

9. The composition according to claim 1, wherein the composition does not comprise DMSO, or,
wherein the composition comprises DMSO at a concentration of from about 1% to about 10% prior to freezing, or wherein the composition comprises DMSO at a concentration of from about 1% to about 5% prior to freezing.

10. The composition according to claim 1, wherein the population of cells comprises mammalian cells.

11. The composition according to claim 1, wherein the composition is in a frozen state at a temperature of between about 10° C. to about 100° C., or between about 20° C. to about 90° C., or between about 40° C. to about 60° C.

12. The composition according to claim 1, wherein the composition is a lyophilized composition.

13. The composition according to claim 1, wherein the composition contains less than about 90% (vol/vol) of water.

14. A method of producing a population of frozen cells, comprising a step (a):
(a) freezing a composition as defined in claim 1 to produce the population of frozen cells.

15. A method of producing a population of reconstituted viable cells, comprising a step (a) and a step (b):
(a) freezing a composition as defined in claim 1 to produce a population of frozen cells; and
(b) resuspending the population of frozen cells in a reconstitution agent to form a reconstituted composition, wherein at least about 1% of the cells are viable.

16. A method of producing a population of lyophilized cells, comprising a step (a) and a step (b):
(a) freezing a composition as defined in claim 1; and
(b) removing at least about 10% (vol/vol) of water from the frozen composition to produce the population of lyophilized cells.

17. A method of producing a population of reconstituted viable cells, comprising a step (a), a step (b) and a step (c):
(a) freezing a composition as defined in claim 1;
(b) removing at least about 10% (vol/vol) of water from the frozen composition to produce the population of lyophilized cells, and
(c) resuspending the population of lyophilized cells in a reconstitution agent to form a reconstituted composition, wherein at least about 1% of the cells are viable.

18. The method of claim 1, wherein prior to the freezing in step (a), the population of cells is isolated and contacted with a poration solution.

19. The method of claim 1, wherein the poration solution comprises trehalose.

20. The method of claim 19, wherein the concentration of trehalose in the poration solution is from about 0.1 M to about 1.0 M, or from about 0.1 M to about 0.6 M.
